(19) United States
(12) Patent Application Publication
(10) Pub. No.: US 2022/0238042 A1
(43) Pub. Date: Jul. 28, 2022

(54) ENGINEERED BONE MARROW MODEL

(71) Applicant: Oregon Health & Science University, Portland, OR (US)

(72) Inventors: Negin Mokhtari, Portland, OR (US); Danielle Konetski, Portland, OR (US); Keith Beadle, West Linn, OR (US); Anthony Tahayeri, Sherwood, OR (US); Elie Traer, Portland, OR (US); Luiz Bertassoni, Portland, OR (US); Yu-Jui Chiu, Portland, OR (US); Jesus Bueno Alvarez, Portland, OR (US); Raviraj Thakur, Portland, OR (US)

(73) Assignee: Oregon Health & Science University, Portland, OR (US)

(21) Appl. No.: 17/610,401

(22) PCT Filed: May 8, 2020
(86) PCT No.: PCT/US2020/032174

§ 371 Date: Nov. 10, 2021

Related U.S. Application Data
(60) Provisional application No. 62/846,484, filed on May 10, 2019.
(60) Provisional application No. 62/866,237, filed on Jun. 25, 2019.
(60) Provisional application No. 63/021,595, filed on May 7, 2020.

(51) Int. Cl. G09B 23/30; G01N 33/50

(52) U.S. Cl. CPC G09B 23/306; G01N 33/5088

(57) ABSTRACT

The present invention provides engineered models that recapitulate bone marrow, including the endosteal and vascular niches, and bone marrow diseases. The models can be implemented in many ways including, but not limited to, on a microfluidic device or microfluidic chip, a culture model in vitro, and as an implantable model in vivo. Also provided are methods of using these models, including high-throughput systems and in personalized medicine analysis. Also provided are devices for use in the provided bone marrow models, and kits that include at least one such device.

### CROSS-REFERENCE TO RELATED APPLICATIONS

This is the U.S. National Phase of International Patent Application No. PCT/US2020/032174, filed May 8, 2020, which claims priority to and the benefit of the earlier filing date of U.S. Provisional Patent Application No. 62/846,484, filed May 10, 2019, and to U.S. Provisional Patent Application No. 62/866,237, filed Jun. 25, 2019, and to U.S. Provisional Patent Application No. 63/021,595, filed May 7, 2020; the contents each of these prior applications are incorporated herein in their entirety.

### FIELD OF THE DISCLOSURE

This invention concerns model engineered bone marrow systems that mimic the structure and function of the bone marrow and bone marrow diseases, and their implementations. The systems are useful for instance in the studies related to drug screening, agent toxicity, personalized medicine, biomarker discovery, disease progression, and drug and radiation side effects, such as myelosuppression and metastasis.

### BACKGROUND OF THE DISCLOSURE

The maintenance and regulation of stem cells in the bone marrow, as well as their differentiation, is achieved through complex structural, physical, molecular and cellular cues. Even though there have been numerous attempts to recreate this environment in vivo and in vitro, none of the current models have been able to mimic this structure to provide prolonged and efficient culture and survival of the bone marrow cells. Thus, there is a need for an advanced culture system that can mimic the physiological and biological complexity that is seen in the bone marrow.

Current in vivo models that attempt to recreate the bone marrow in mice are time consuming and expensive and thus, not optimal for clinical translation (Reinisch et al., Nat Med. 22(7):812-821, 2016; Shih et al., PNAS USA 114(2):5419-5424, 2017). Current in vitro models of the bone marrow do not fully replicate the different niches in the marrow (Torisawa et al., Nature Meth. 11(6)663-669, 2014; Sieber et al., J Tissue Eng Regen Med. 12(2):479-489, 2018) and lack prolonged culture of bone marrow stem cells. Moreover, they are time consuming and fail to fully simulate the physical cues of the bone marrow and the specific tissue interface, microenvironmental control, etc.

There remains a need for advanced culture systems that can mimic the physiological and biological complexity seen in native bone marrow.

### SUMMARY OF THE DISCLOSURE

Provided herein are systems, methods, and means to synthesize an engineered bone marrow that recreates the different niches of bone tissue. Models can be implemented in a two- or three-dimensional architecture and include or not include cells, which can be seeded directly or pre-encompassed or pre-encapsulated in a hydrogel structure, or any combination thereof. The provided engineered models recreate the bone marrow's niche architecture, including the endosteal and vascular niche. The engineered models also provide methods and means for introducing compounds or materials of interest to one or both niches for testing purposes.

The present disclosure provides an engineered model recapitulating the bone marrow, including the endosteal and vascular niches, and bone marrow diseases. The model can be implemented in many ways including, but not limited to, on a microfluidic device or microfluidic chip, a culture model in vitro, or as an implantable in vivo model.

The present disclosure provides bone marrow models that mimic the key compositional and structural hallmarks as well as biological function of either healthy human bone marrow or those modeling disease states, such as hematologic malignancies in vitro in real time. Also provided are organ-on-a-chip microdevices that recapitulate the endosteal and vascular niches of bone marrow to more accurately reproduce the hematopoietic response of bone marrow in a controlled system that allows for real-time detection of events relevant to marrow function and cancer progression.

Provided herein is a bone marrow model including: a first microenvironment including a hydrogel with or without cells; and a second microenvironment including: a hydrogel with cells found in a normal hematopoietic niche of normal bone marrow; or a hydrogel with cells found in a condition selected from the group consisting of hematologic malignancies, marrow failure syndromes, pre-malignant clonal hematopoiesis, and solid tumor metastases to bone marrow.

Another embodiment is a bone marrow model including: a first microenvironment including a hydrogel with mature bone cells; and a second microenvironment including a hydrogel with cells found in a hematopoietic niche of normal bone; wherein the first microenvironment and second microenvironment are maintained in a position where they can interact with a fluid medium.

Yet another embodiment is a bone marrow model including: a first microenvironment including a hydrogel with immature bone cells; and a second microenvironment including a hydrogel with cells found in a hematopoietic niche of normal bone; wherein the first microenvironment and second microenvironment are maintained in a position where they can interact with a fluid medium.

Also provided is a bone marrow model including: a first microenvironment including a hydrogel without cells or with mature bone cells and/or mesenchymal stem cells; and a second microenvironment including a non-mineralized hydrogel incorporating elements of a hematopoietic niche; wherein the first microenvironment is in contact with the second microenvironment.

Another provided embodiment is a bone marrow model including: a first microenvironment including a hydrogel with mature bone cells; and a second microenvironment including: a hydrogel with cells found in a normal hematopoietic niche of normal bone marrow; or a hydrogel with cells associated with a diseased state in a hemopoietic niche.

Yet another embodiment is a bone marrow model including: a first microenvironment including a hydrogel with immature bone cells and/or mesenchymal stem cells; and a second microenvironment including: a hydrogel with cells found in a normal hematopoietic niche of normal bone marrow; or a hydrogel with cells associated with a diseased state in a hemopoietic niche.

Also provided is a bone marrow model including: a first microenvironment including a mineralized hydrogel without cells or with mature bone cells and/or mesenchymal stem cells; and a second microenvironment including a non-mineralized hydrogel incorporating elements of a hematopoietic niche; wherein the first microenvironment is in contact with the second microenvironment.

Another embodiment is a bone marrow model including: a first microenvironment including a mineralized hydrogel without cells or with bone cells or mesenchymal stem cells; and is implemented as mineralized fragments in the form of shards; and a second microenvironment including a hydrogel with cells found in the hematopoietic niche of normal, modified, or diseased bone marrow; wherein the mineralized hydrogel of the first microenvironment is embedded in the hydrogel of the second microenvironment and the hydrogels of both the first and second microenvironment s are maintained in a position where they can interact with a fluid medium.

Also provided is a bone marrow model including: a) a first microenvironment including a mineralized hydrogel without cells or with mature bone cells or mesenchymal stem cells; and b) a second microenvironment including a hydrogel material with cells found in the hematopoietic niche of normal, modified, or diseased bone marrow; wherein the layers of hydrogel referenced above a) is embedded in b) and a) and b) are maintained in a position where they can interact with a fluid medium.

In yet another embodiment, there is provided a microfluidic bone marrow model including: a central chamber open at two opposing ends and defined by continuous walls formed by an upper plate, a lower plate, and two side plates; wherein the upper plate and lower plate are substantially parallel to each other, are separated from one another by the height of the central chamber, and are substantially perpendicular to the two side plates; and wherein the two side plates are substantially parallel to each other, are separated from one another by the width of the central chamber, and are substantially perpendicular to the top plate and the bottom plate; and a permeable barrier dividing the central chamber parallel to and maintained between the upper plate and lower plate and in contact with each of the two side plates, the permeable barrier dividing the central chamber into an upper chamber located between the permeable barrier and upper plate and a lower central chamber located between the permeable barrier and the lower plate; wherein the lower central chamber is divided into a first microenvironment including an endosteal niche and a second microenvironment including a stem cell niche, the stem cell niche being in contact with the permeable barrier and the two side plates and the endosteal niche being in contact with the bottom plate and the two side plates; and further wherein the stem cell niche and the endosteal niche are in contact and communication with each other.

Provided in yet another embodiment is a method of testing a drug or drug candidate, or a pharmaceutically acceptable salt thereof, using a bone marrow model as described herein, the method including: ascertaining the elements of a device or design described in the bone marrow model having first microenvironment and a second microenvironment; exposing at least one of the first microenvironment and the second microenvironment to the drug or drug candidate, or a pharmaceutically acceptable salt thereof; and ascertaining any changes to the elements of the device or design following exposure to the drug or drug candidate, or a pharmaceutically acceptable salt thereof.

A system for use in a bone marrow model is also provided, which system includes: a well plate including: an array of media wells configured to receive fluid media, a media well of the array of media wells including a bottom end that is covered with a permeable barrier; an array of hydrogel chambers, a hydrogel chamber of the array of hydrogel chambers positioned underneath the permeable barrier; and an array of loading ports, a loading port of the array of loading ports in fluid communication with the hydrogel chamber to direct a hydrogel containing cells into the hydrogel chamber.

Yet another embodiment is a system for use in a bone marrow model, the system including: a well plate including: a first media well and a second media well, the first media well and the second media well configured to receive fluid media, wherein a first bottom end of the first media well is covered with a permeable membrane, and wherein a second bottom end of the second media well is covered with the permeable membrane, or a different permeable membrane; a first extracellular matrix (ECM) chamber and a second ECM chamber, wherein the first ECM chamber is positioned underneath the first media well with the permeable membrane interposed between the first ECM chamber and the first media well, and wherein the second ECM chamber is positioned underneath the second media well with the permeable membrane, or the different permeable membrane, interposed between the second ECM chamber and the second media well; and a first loading port and a second loading port, wherein the first loading port is in fluid communication with the first ECM chamber, and wherein the second loading port is in fluid communication with the second ECM chamber.

Also provided are methods that include: expressing, into an array of loading ports in a well plate, a hydrogel containing cells to at least partially fill an array of hydrogel chambers in the well plate with the hydrogel; and filling, at least partially, an array of media wells in the well plate with fluid media, wherein a media well of the array of media wells includes a bottom end that is covered with a permeable barrier, wherein a hydrogel chamber of the array of hydrogel chambers is positioned underneath the permeable barrier, and wherein the cells include one or more of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages, hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, osteoblast precursor cells, osteolineage cells, chondrocyte precursor cells, mesenchymal stem and progenitor cells or mesenchymal stromal progenitor cells, CXCL12-abundant reticular cells, and exosomes.

Another embodiment is a system for use in a bone marrow model, including: a first reservoir including: an array of microfluidic channels, a microfluidic channel of the array of microfluidic channels configured to receive fluid media at an inlet of the microfluidic channel and allow the fluid media to egress from an outlet of the microfluidic channel; and an array of inserts, an insert of the array of inserts coupled to at least one of the inlet of the microfluidic channel or the outlet of the microfluidic channel and including: a media channel to receive the fluid media at an inlet of the media channel and allow the fluid media to egress from an outlet of the media channel; a hydrogel chamber positioned underneath the media channel, the hydrogel chamber configured to be filled, at least partially, with a hydrogel containing cells; and a permeable barrier interposed between the hydrogel chamber and the media channel; and a second reservoir configured to detachably couple to a bottom of the first reservoir, the second reservoir including: an array of collection wells, a collection well of the array of collection wells positioned underneath the microfluidic channel when the second reservoir is coupled to the bottom of the first reservoir and configured to collect the fluid media that has passed through the microfluidic channel and through the insert.

Also described is a system for use in a bone marrow model, the system including: a first reservoir including: a first microfluidic channel and a second microfluidic channel, the first microfluidic channel and the second microfluidic channel each configured to allow fluid media to pass therethrough; and a first insert and a second insert, the first insert coupled to at least one of an inlet of the first microfluidic channel or an outlet of the first microfluidic channel, and the second insert coupled to at least one of an inlet of the second microfluidic channel or an outlet of the second microfluidic channel, the first insert and the second insert each including: a media channel to receive the fluid media at an inlet of the media channel and allow the fluid media to egress from an outlet of the media channel; an extracellular matrix (ECM) chamber positioned underneath the media channel, the ECM chamber configured to be filled, at least partially, with an ECM; and a permeable barrier interposed between the ECM chamber and the media channel; and a second reservoir including: a first collection well and a second collection well, wherein the first collection well is vertically-aligned with the first microfluidic channel and the second collection well is vertically-aligned with the second microfluidic channel when the second reservoir is positioned underneath the first reservoir.

A vacuum insert for use in a bone marrow model is also described, including: an annular base including: a plurality of inlets defined in a top of the annular base; a media channel defined in an interior of the annular base and in fluid communication with the plurality of inlets; and a permeable barrier positioned underneath the media channel; and a central tube coupled to the annular base at a center of the annular base and extending orthogonally from the top of the annular base, wherein the vacuum insert is configured to couple to a vacuum source at a top end of the central tube, and wherein the media channel of the annular base is configured to allow fluid media drawn into the vacuum insert via the plurality of inlets to pass through the annular base and into the central tube.

Another vacuum insert for use in a bone marrow model embodiment includes: an annular base including: a one or more inlets defined in a top of the annular base; a media channel defined in an interior of the annular base and in fluid communication with the one or more inlets; and a permeable barrier positioned underneath the media channel; and a central tube at a center of the annular base and extending orthogonally from the top of the annular base, wherein the vacuum insert is configured to couple to a vacuum source at a top end of the central tube, and wherein the central tube is in fluid communication with the media channel.

In yet another embodiment, there is provided a microfluidic chip for use in a bone marrow model, which microfluidic chip includes: a first substrate including: an inlet defined in a top of the first substrate, the inlet configured to allow fluid media to ingress into the microfluidic chip; an outlet defined in the top of the first substrate, the outlet configured to allow the fluid media to egress from the microfluidic chip; and a loading port defined in the top of the first substrate, the loading port configured to receive a hydrogel containing cells; a second substrate disposed underneath the first substrate, the second substrate including: a hydrogel channel configured to be filled, at least partially, with the hydrogel containing cells, wherein an end of the hydrogel channel is vertically-aligned with the loading port; a first through-hole that is vertically-aligned with the inlet; and a second through-hole that is vertically-aligned with the outlet; a permeable barrier disposed underneath the second substrate; and a third substrate disposed underneath the permeable barrier, the third substrate including: a media channel configured to receive the fluid media, wherein a first end of the media channel is vertically-aligned with the first through-hole and a second end of the media channel is vertically-aligned with the second through-hole.

Also provided is a device for use in a bone marrow model, the device including: a top substrate including: an inlet defined in a top of the top substrate, the inlet configured to allow fluid media to ingress into the device; an outlet defined in the top of the top substrate, the outlet configured to allow the fluid media to egress from the device; and a loading port defined in the top of the top substrate, the loading port configured to receive a hydrogel containing cells; a bottom substrate including a media channel configured to receive the fluid media; an intermediate substrate interposed between the top substrate and the bottom substrate, the intermediate substrate including: a hydrogel channel configured to be filled, at least partially, with the hydrogel containing cells, wherein an end of the hydrogel channel is vertically-aligned with the loading port; a first through-hole that is vertically-aligned with the inlet and with a first end of the media channel; and a second through-hole that is vertically-aligned with the outlet and with a second end of the media channel; and a permeable barrier interposed between the intermediate substrate and the bottom substrate.

It is understood that a microenvironment referred to as “with cells” herein is understood to include a hydrogel encompassing, encapsulating, enclosing, incorporating, holding, containing, or otherwise supporting cells, as well as a hydrogel having cells on one or more internal or external surfaces, including a hydrogel surface coated with or supporting cells.

### BRIEF DESCRIPTION OF THE DRAWINGS

FIG. 1 illustrates a perspective view of an example well plate that may constitute a system, according to embodiments disclosed herein.

FIG. 2 illustrates a front view of the example well plate of FIG. 1, FIG. 2 showing internal features of the well plate in dashed lines.

FIG. 3 illustrates a side view of the example well plate of FIG. 1, FIG. 3 showing internal features of the well plate in dashed lines.

FIG. 4 illustrates a top view of the example well plate of FIG. 1, FIG. 4 showing internal features of the well plate in dashed lines.

FIG. 5 illustrates a top view of a bottom substrate of the example well plate of FIG. 1, the bottom substrate including recessed areas that form hydrogel chambers when the well plate is assembled.

FIG. 6 illustrates a cross-sectional view of a portion of the example well plate of FIG. 1, taken along section line A-A.

FIG. 7 illustrates the cross-sectional view of FIG. 6, but with hydrogel in the hydrogel chambers, and with fluid media in the media wells.

FIG. 8 illustrates a perspective view of an example second well plate that is configured to detachably couple to a top of the example well plate of FIG. 1, according to embodiments disclosed herein.

FIG. 9 illustrates a front view of the example second well plate of FIG. 8, FIG. 9 showing internal features of the second well plate in dashed lines.

FIG. 10 illustrates a side view of the example second well plate of FIG. 8, FIG. 10 showing internal features of the second well plate in dashed lines.

FIG. 11 illustrates a top view of the example second well plate of FIG. 8, FIG. 11 showing occluded and/or internal features of the second well plate in dashed lines.

FIG. 12 illustrates a method of detachably coupling the example second well plate of FIG. 8 to a top of the example (first) well plate of FIG. 1, FIG. 12 showing internal features of both well plates in dashed lines.

FIG. 13 illustrates a perspective view of a system including the example second well plate of FIG. 8 coupled to the example (first) well plate of FIG. 1.

FIG. 14 illustrates a side view of the example system of FIG. 13, FIG. 14 showing internal features of the system in dashed lines.

FIG. 15 illustrates a cross-sectional view of the example system of FIG. 13, taken along section line B-B.

FIG. 16 illustrates a zoomed in view of a portion of the cross-sectional view of FIG. 15, FIG. 16 showing the system operating in a continuous perfusion mode of operation.

FIG. 17 illustrates a flow diagram of an example process of using a well plate system for cell culturing.

FIG. 18 illustrates a perspective view of an example system including a first reservoir and a second reservoir, according to embodiments disclosed herein.

FIG. 19 illustrates a front view of the example first reservoir of FIG. 18, according to one embodiment where an array of microfluidic channels is in the form of spiral channels, FIG. 19 showing internal features of the first reservoir in dashed lines.

FIG. 20 illustrates a side view of the example first reservoir of FIG. 18, according to the spiral channel embodiment, FIG. 20 showing internal features of the first reservoir in dashed lines.

FIG. 21 illustrates a top view of the example first reservoir of FIG. 18, according to the spiral channel embodiment, FIG. 21 showing internal features of the first reservoir in dashed lines.

FIG. 22 illustrates a perspective view and a top view of a microfluidic channel of the first reservoir in the form of a spiral channel.

FIG. 23 illustrates a front view of the example first reservoir FIG. 18, according to another embodiment where an array of microfluidic channels is in the form of serpentine channels, FIG. 23 showing internal features of the first reservoir in dashed lines.

FIG. 24 illustrates a side view of the example first reservoir of FIG. 18, according to the serpentine channel embodiment, FIG. 24 showing internal features of the first reservoir in dashed lines.

FIG. 25 illustrates a top view of the example first reservoir of FIG. 18, according to the serpentine channel embodiment, FIG. 25 showing internal features of the first reservoir in dashed lines.

FIG. 26 illustrates a perspective view and a top view of a microfluidic channel of the first reservoir in the form of a serpentine channel.

FIG. 27 illustrates views of an insert that may be coupled to an inlet or an outlet of a microfluidic channel of the first reservoir, the insert including a hydrogel chamber positioned underneath a media channel with a permeable barrier interposed therebetween.

FIG. 28 illustrates a perspective view of the example second reservoir of FIG. 18, according to embodiments disclosed herein.

FIG. 29 illustrates a front view of the example second reservoir of FIG. 28, FIG. 29 showing internal features of the second reservoir in dashed lines.

FIG. 30 illustrates a side view of the example second reservoir of FIG. 28, FIG. 30 showing internal features of the second reservoir in dashed lines.

FIG. 31 illustrates a top view of the example second reservoir of FIG. 28, FIG. 31 showing internal features of the second reservoir in dashed lines.

FIG. 32 illustrates a perspective view of vacuum insert, according to embodiments disclosed herein.

FIG. 33 illustrates a cross-sectional view of the example vacuum insert of FIG. 32, taken along section line D-D, with the vacuum insert disposed inside of a media well.

FIG. 34 illustrates a perspective exploded view and a perspective assembled view of an example microfluidic chip, according to embodiments disclosed herein.

FIG. 35 illustrates a cross-sectional view of the example microfluidic chip of FIG. 34, taken along section line C-C.

FIG. 36 illustrates the formation and mineralization of a cell-laden hydrogel of the type useful in designs and methods herein. Reproduced from Thrivikraman et al. (Nature Comm., 10(1):3250, 2019).

FIG. 37 provides a step-by-step illustration of a ring model bone marrow preparation.

FIG. 38 is a photograph of a ring model bone marrow preparation showing a mineralized endosteal niche outer core and non-mineralized vascular niche core.

FIG. 39 depicts a layered bone marrow chip design. It will be understood that the order of the layers in the chip can be reversed.

FIG. 40 depicts a separate two-layered niche design.

FIG. 41 illustrates the elements and arrangement of a microfluidics device design.

FIG. 42 illustrates a design containing an endosteal niche, a vascular niche, and two media channels.

FIG. 43 illustrates another design in which the endosteal niche and vascular niche are side-by-side and each is associated with a different media channel.

FIG. 44. Illustrates a further design in which both niches are separated from the same media channel by a permeable barrier.

FIG. 45 is a lateral cross-sectional illustration of a device formed using spherical endosteal niche forms within the hematopoietic niche microenvironment.

FIG. 46 depicts a production sequence for a ring design for implantation into a test animal.

FIG. 47 provides a three-dimensional partial overhead view of a sandwich (HERO™) chip design.

FIG. 48A-48C. In Vivo Model Bone marrow organoid was created in vitro and implanted subcutaneously into NGS mouse. After 8 weeks the organoid was removed and stained with hematoxylin and eosin. Organoids where composed of bmMSC with or without shards & disks suspended in Matrigel. and HSC suspended in Matrigel. After 8 weeks the organoid was removed and stained with hematoxylin and eosin. FIG. 48A shows endosteal free gel, FIG. 48B (plus inset) shows gel containing a mineralized disk. FIG. 48C shows gel containing mineralized shards.

### DETAILED DESCRIPTION OF THE DISCLOSURE

Another embodiment provides a bone marrow model including: a first microenvironment including a mineralized hydrogel with mature bone cells; and a second microenvironment including a hydrogel with cells found in the hematopoietic niche of normal (or diseased) bone marrow; wherein the first microenvironment and second microenvironment are maintained in a position where they can interact with a fluid medium.

Another embodiment provides a bone marrow model including: a first microenvironment including a mineralized hydrogel with immature bone cells; and a second microenvironment including a hydrogel with cells found in the hematopoietic niche of normal (or diseased) bone marrow; wherein the first microenvironment and second microenvironment are maintained in a position where they can interact with a fluid medium.

Yet another embodiment provides a bone marrow model including: a first microenvironment including a mineralized hydrogel without cells or with mature bone cells and/or mesenchymal stem cells; and a second microenvironment including a non-mineralized hydrogel incorporating elements of the hematopoietic niche in normal bone; wherein the first microenvironment is in contact with the second microenvironment. Optionally, the first and second microenvironments are indirectly in contact with each other, in that a permeable layer is present between them.

Another embodiment provides a bone marrow model including: a first microenvironment including a mineralized hydrogel without cells or with mature bone cells and/or mesenchymal stem cells; and a second microenvironment including a non-mineralized hydrogel incorporating elements of the normal (or diseased) hematopoietic niche; wherein the first microenvironment is in contact with the second microenvironment.

In the embodiments herein, the first microenvironment including a mineralized or unmineralized hydrogel with no cells or with mature or immature bone cells or mesenchymal stem cells may also be referred to as the “endosteal niche.” Similarly, the microenvironment including a non-mineralized hydrogel incorporating elements of a natural or diseased hematopoietic niche may be referred to as the “hematopoietic niche,” the “vascular niche,” or the “stem cell niche.”

In some embodiments, the first microenvironment (endosteal niche) and the second microenvironment (the hematopoietic, vascular, or stem cell niche) are in substantial contact with each other.

Yet another embodiment provides a microfluidic device including: a central chamber open at two opposing ends (for instance, the top and bottom in some embodiments) and defined by continuous walls formed by an upper plate, a lower plate, and two side plates; wherein the upper plate and lower plate are substantially parallel to each other, are separated from one another by the height of the central chamber, and are substantially perpendicular to the two side plates; and wherein the two side plates are substantially parallel to each other, are separated from one another by the width of the central chamber, and are substantially perpendicular to the top plate and the bottom plate; and a permeable barrier dividing the central chamber parallel to and maintained between the upper plate and lower plate and in contact with each of the two side plates, the permeable barrier dividing the central chamber into and upper chamber located between the permeable barrier and upper plate and a lower central chamber located between the permeable barrier and the lower plate; wherein either the upper or the lower central chamber is divided into a first microenvironment including an endosteal niche and a second microenvironment including a stem cell niche, the stem cell niche being in contact with the permeable barrier and the two side plates and the endosteal niche being in contact with the bottom plate and the two side plates; and further wherein the stem cell niche and the endosteal niche are in contact and communication with each other.

In embodiments, the material in the lower central chamber including the stem cell niche is in substantial contact with the material including the endosteal niche. In other embodiments, the material in the lower central chamber including the stem cell niche is in substantial contact with the permeable barrier.

In yet another embodiment, a bone marrow model is provided including: a first microenvironment including a mineralized hydrogel without cells or with bone cells or mesenchymal stem cells; and is implemented as mineralized fragments in the form of shards; and second microenvironment including a hydrogel with cells found in the hematopoietic niche of normal, modified or diseased bone marrow; wherein the hydrogel of the first microenvironment is embedded in the hydrogel of the second microenvironment and the hydrogels are maintained in a position where they can interact with a fluid medium.

Also provided are examples of the bone marrow models, which include: a first microenvironment including a mineralized hydrogel without cells or with mature bone cells or mesenchymal stem cells; wherein the mineralized hydrogel is in a form selected from the group of spheres, spheroids, beads, and droplets; and a second microenvironment including a hydrogel material with cells found in the hematopoietic niche of normal, modified or diseased bone marrow; wherein the layers of hydrogel is embedded in are maintained in a position where they can interact with a fluid medium.

Additional exemplary embodiments are provided, wherein the first microenvironment including a mineralized hydrogel further includes diseased cells, and the non-mineralized hydrogel microenvironment does not contain diseased cells. Also provided are embodiments both the first microenvironment including a mineralized hydrogel and the non-mineralized hydrogel microenvironment contain diseased cells.

Another embodiment provides a bone marrow model including: a mineralized hydrogel microenvironment; a non-mineralized hydrogel microenvironment; means for directing one or more fluid mediums; and an architecture that maintains the mineralized microenvironment and the non-mineralized microenvironment in positions in which they can interact with the one or more fluid mediums and/or one or more microenvironment s that contain cells.

Also further embodiment provides a bone marrow model including: a mineralized hydrogel microenvironment; a non-mineralized hydrogel microenvironment; means for directing one or more fluid mediums; one or more interfaces between the mineralized hydrogel microenvironment and the non-mineralized hydrogel microenvironment; and an architecture that maintains the mineralized microenvironment and the non-mineralized microenvironment in positions in which they can interact with the one or more fluid mediums and/or one or more microenvironment s that contain cells.

Also provided is a bone marrow model including: a first microenvironment including a hydrogel without cells or with mesenchymal stem cells; and a second microenvironment including: a hydrogel with cells found in a normal hematopoietic niche of normal bone marrow; or a hydrogel with cells associated with a diseased state in a hemopoietic niche.

Also provided is a bone marrow model including: a first microenvironment including a mineralized hydrogel without cells or with mesenchymal stem cells (MSCs, such as MSCs desired from a patient); and a second microenvironment including: a hydrogel with cells found in a normal hematopoietic niche of normal bone marrow; or a hydrogel with cells associated with a diseased state in a hemopoietic niche.

Another provided embodiment is a bone marrow model including: a first microenvironment including a mineralized hydrogel without cells or with mature bone cells and/or mesenchymal stem cells; and a second microenvironment including a non-mineralized hydrogel incorporating elements of the hematopoietic niche; wherein the first microenvironment is in contact with the second microenvironment. Optionally, the first and second microenvironments are indirectly in contact with each other, in that a permeable layer is present between them.

Additionally provided is a bone marrow model including: a first microenvironment including a mineralized hydrogel without cells or with mature bone cells and/or mesenchymal stem cells; and a second microenvironment including a non-mineralized hydrogel incorporating elements of the hematopoietic niche in a diseased state; wherein the first microenvironment is in contact with the second microenvironment.

Additionally provided is a bone marrow model including: a first microenvironment including a mineralized hydrogel without cells or with mature bone cells and/or mesenchymal stem cells and incorporating elements of an endosteal niche in a diseased state; and a second microenvironment including a non-mineralized hydrogel incorporating elements of the hematopoietic niche; wherein the first microenvironment is in contact with the second microenvironment.

Additionally provided is a bone marrow model including: a first microenvironment including a mineralized hydrogel without cells or with mature bone cells and/or mesenchymal stem cells and incorporating elements of an endosteal niche in a diseased state; and a second microenvironment including a non-mineralized hydrogel incorporating elements of the hematopoietic niche in a diseased state; wherein the first microenvironment is in contact with the second microenvironment.

In some embodiments, the mineralized hydrogels for the endosteal niches or microenvironment s of the devices herein are prepared in bulk and then partitioned or divided into fragments of desired size for one or more devices. In other embodiments, the hydrogels are created and partitioned or divided into fragments of desired size prior to mineralization. Embodiments in each of these methods of preparation include those in which the final mineralized segment has a length of from about 5 micrometers to about 1,000 micrometers along the segment's longest axis.

In some embodiments, the mineralized and non-mineralized microenvironment s/niches are prepared separately before being joined together. In other embodiments, the two microenvironments are joined prior to mineralization of one microenvironment.

In some embodiments, the endosteal and vascular microenvironment environment is recreated in an in-vitro environment by a biomimetic engineered bone marrow including a mineralized compartment encapsulating bone cells (endosteal niche) and an inner soft compartment incorporating the elements of hematopoietic niche (vascular niche).

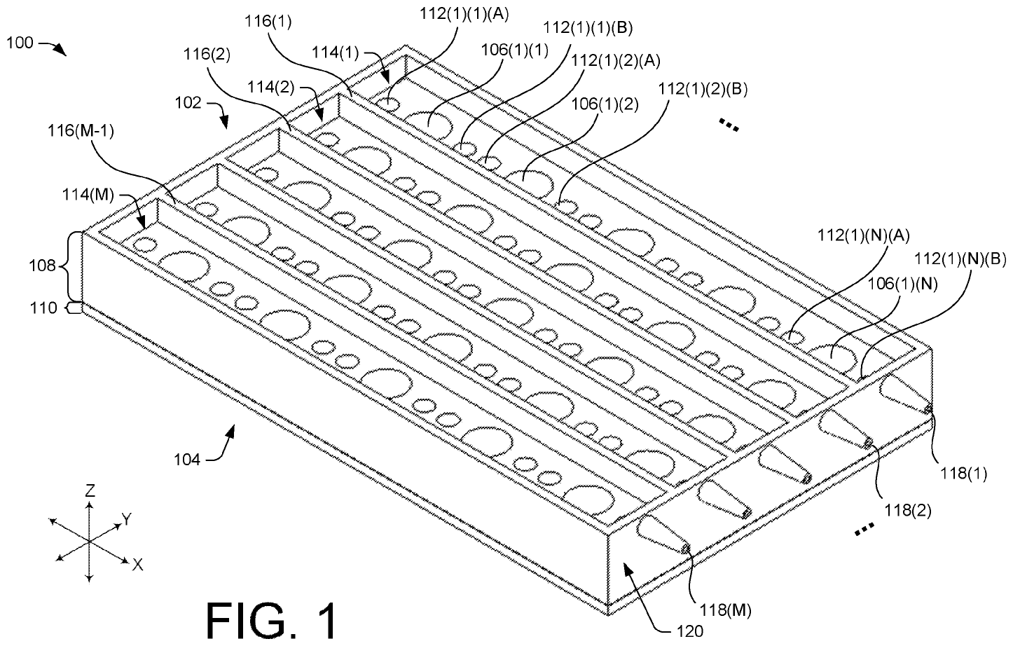

The mineralized endosteal niche or microenvironment can be recapitulated in a variety of ways including but not limited to mineralized spheres, mineralized shards, and mineralized layer/sheets, as outlined in FIG. 1.

As also depicted, the endosteal and vascular niches/microenvironment s can be interfaced in a variety of ways such as layer by layer or the vascular niche can for example be embedded with different geometries such as spheres/beads or shards (FIG. 1).

Models or designs herein can be in 2D or 3D, with cells (healthy, disease, or modified or bone marrow aspirates) including but not limited to Osteoblasts, osteocytes, osteoclasts, Mesenchymal Stem Cells (MSCs) (such as MSCs from a diseased subject), Hematopoietic stem cells (HSCs), stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages or without cells, seeded with or encapsulating cells or any combination thereof. The cells may be of human or animal source, including primary or immortalized cell lines. The cells may be from diseased subjects (and in such instances may be referred to as “diseased cells”). The present invention, and each method and design herein, is not limited to a particular cell type or cell origin or to a particular type of matrixes that is used.

Models and designs of this invention can be implemented in vitro, including but not limited to static or cultured in vitro in a variety of geometries including but not limited to disk, ring, and shards, with or without flow for applications including, but not limited to, high throughput screening, or on different microfluidic devices, or in vivo including but not limited to mouse models in a variety of geometries including but not limited to disk and ring (FIG. 1).

The in vitro bone marrow model or the disease model can be cultured with or without flow to investigate a myriad of subjects including, but not limited to, high throughput screening, radiation, and toxicology.

The in vitro bone marrow models or the diseased bone marrow models herein may be cultured in a static culture manner, such as trans-wells or multi-well plates to investigate a myriad of subjects including, but not limited to, high throughput screening, radiation, and toxicology.

The in vitro bone marrow models or the diseased bone marrow models herein may be cultured in trans-wells or multi-well plates supplied with fluid flow or built in channels that supply fluid flow to investigate the myriad of subjects described herein.

The in vitro bone marrow model such as bone marrow on microfluidic devices can be connected to other organ chips. For instance, the microfluidic device can be connected to other organ chips including but not limited to breast, liver, etc. to investigate a myriad of subjects including but not limited to metastasis, drug screening, radiation and toxicology.

Different models of implementation of the bone marrow can be used to investigate a myriad of subjects, including but not limited to: disease progression (such as in early detection of cancer), therapeutics, drug screening, toxicology, radiation/radiotherapy and drug side effects, safety and risk assessment, efficacy, engraftment, migration, metastasis, organ function assessment, drug and biomarker discovery, combination therapy, drug-drug interaction, and bone autograft and allograft techniques.

The structure of the microfluidic devices and implantable units herein may include of any material biologically acceptable for the use. In some embodiments, they include a polymer/plastic material, such as Cyclic Olefin Polymer (COPs), Cyclic Olefin co-Polymers (COCs), polydimethylsiloxane (PDMS), acrylic, silicon, polyetherimide copolymers (PEI, ULTEM®), acetal copolymers, polyethersulfone (PES, RADEL A®), polyarylethersulfone (PAES, RADEL R®), polycarbonate, ultra-high molecular weight polyethylene, polyetheretherketone (PEEK), polyglycolic acid (PGAA) and its copolymers, poly(methyl methacrylate) (PMMA), polyphenylsulfone (PPSU), polyaryletherketone (PEEK-OPTIMA®), poly(methyl methacrylate) (PMMA), Polypropylene (PP), Polystyrene (PS), High impact polystyrene (HIPS), Acrylonitrile butadiene styrene (ABS), Polyethylene terephthalate (PET), Amorphous PET (APET), Polyester (PES), Fibers, textiles, Polyamides (PA), Nylons, Poly(vinyl chloride) (PVC), Polyurethanes (PU), Polycarbonate (PC), Polyvinylidene chloride (PVDC) (Saran), Polyvinylidene Fluoride (PVDF), Polyethylene (PE), polyacetic acid (PLA), poly methyl(pentene) (PMP) Polytetrafluoroethylene (PTFE, trade name Teflon), Fluorinated ethylene propylene (FEP), (Polyetherketone), Phenolics (PF), (phenol formaldehydes), Perfluoroalkoxy (PFA), Poly(methyl methacrylate) (PMMA), Urea-formaldehyde (UF), Melamine formaldehyde (MF), Polylactic acid and Plastarch material or any mixture thereof.

In the models and designs herein, the microenvironment referred to as a hematopoietic niche, a vascular niche, or hematopoietic stem cell niche encompasses or contains cells found in the hematopoietic niche of normal bone, such as stem cells. Cells found contained in this microenvironment may include those selected from the group of hematopoietic stem cells (HSCs), long-term hematopoietic stem cells (LT-HSCs), short-term hematopoietic stem cells (ST-HSCs), multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), and other specialized marrow stromal populations such as CXCL12-abundant reticular (CAR) cells.

Diseased cells that may be included in this model include—but not limited to—leukemia [acute myeloid leukemia (AML), chronic myeloid leukemia (CML), atypical CML, chronic neutrophilic leukemia, acute lymphoblastic leukemia (ALL), etc.], multiple myeloma, Non-Hodgkin lymphoma, Chronic lymphocytic leukemia (CLL), Hodgkin lymphoma, T-cell lymphoma, bone marrow failure syndromes, myelodysplastic syndrome (MDS), clonal hematopoiesis of indeterminate potential (CHIP), clonal cytopenias of undetermined significance (CCUS), aplastic anemia, and metastatic solid tumors that travel to the bone marrow (lung, breast, kidney, prostate, thyroid, etc.). It is understood that hemopoietic stem cells found in this microenvironment may be quiescent or proliferating. Normal and malignant cells can be analyzed either directly in the provided models by methods such as immunofluorescence and/or genetic modification/fluorescent cell labeling prior to implantation into the model. Cells can also be collected from the circulating media feeding the model, or with single cell isolation and flow cytometry using normal and malignant markers.

Similarly, the endosteal niche microenvironment in the model designs herein may include cells found in natural endosteal niches, including osteoblasts, osteoprogenitors, and osteochondroprogenitors.

In one embodiment of each design or device described herein, the endosteal niche includes osteoblasts. In other embodiments, the stem cell niche includes mesenchymal stem cells (MSCs) and hematopoietic stem cells (HSCs).

In some embodiments, the endosteal niche includes a mineralized hydrogel, such as a mineralized collagen. In other embodiments, it includes a non-mineralized hydrogel, such as a collagen.

Also provided are methods of using the devices and designs in methods of medical testing. Representative methods include: ascertaining the elements of a device or design described herein having an endosteal niche and a vascular niche; exposing at least one of the endosteal niche and the vascular niche to an external stimulus or factor; and ascertaining any changes to the elements of the device or design following exposure to the external stimulus or factor.

Ascertaining the elements of a device, model, system, or design includes one or more of identifying the concentration of relevant factors, such as cell(s) or biologically active molecule(s), or the state of the structure, function, level of activity, metabolism, respiration, reproduction, development, reaction, etc. of cell(s) or biologically active compound(s) before a test is conducted or an external stimulus is applied to the system and cell(s) or molecule(s) therein.

In some embodiments, the external stimulus or factor includes a media, such as a fluid media, containing one or more test agents, such as a pharmaceutical agent, a toxin, or metastasized or modified cells to be tested or the localized pharmacokinetics or pharmacodynamics thereof to be tested.

In some embodiments, the external stimulus or factor includes such a media containing a natural biological stimulating factor, such as for instance peptide signaling molecules, bone morphogenetic proteins (BMPs), transforming growth factor beta (TGF-β), insulin-like growth factors I and II (IGF-I and IGF-II), platelet derived growth factor (PDGF), vascular endothelial growth factor-A (VEGF) and basic and acidic fibroblast growth factor (bFGF and aFGF).

In some embodiments, the external stimulus or factor includes or is radiation.

Some embodiments provide a test for ascertaining intracellular and/or intercellular activity among the cells present in the endosteal and/or vascular niche. In some embodiments, the test is conducted to ascertain the presence of osteocalcin and bone sialoprotein (BSP) osteogenic markers.

In some embodiments, the test is conducted to ascertain aspects of the survival, absorption, distribution, differentiation, mineralization, metabolism, development, or proliferation of bone cells. In other embodiments, the test is to ascertain the progress of induced pluripotent stem cells (iPSCs) in differentiating to joint tissue cells such as osteoblasts, osteocytes, chondrocytes, tenocytes, fibroblasts, notochordal cells, and/or nucleus pulposus cells.

In further embodiments the tests are conducted to ascertain activities associated with bone and bone marrow metastasis and/or cancer cell migration. In other embodiments, the test is conducted to ascertain the effects of a chemotherapeutic agent. In some embodiments, the tests are conducted to ascertain the effects of pharmacological agents or treatments directed at other organs or tissues have on bone marrow cells or tissues.

It will be understood that the methods of ascertaining the elements discussed above are known in the art for such testing. They include, but are not limited to light or electron microscopic inspection, chemical analysis, immunohistochemical analysis, histology, flow cytometry, colony assays, etc.

### Definitions

All ranges disclosed herein are inclusive of the recited endpoint and independently combinable (for example, the range of “from 2 to 10” is inclusive of the endpoints, 2 and 10, and all the intermediate values). References to a range of claim numbers also includes the claims referenced and each claim between them.

The term “bone marrow” refers to the areas of natural bone containing both hematopoietic stem cells (HSCs) and nonhematopoietic cells. HSCs give rise to all types of mature blood cells, whereas the nonhematopoietic component is composed of osteoblasts/osteoclasts, endothelial cells, endothelial progenitor cells, T lymphocytes, macrophages, mast cells, stromal fibroblasts and mesenchymal stem cells. All these cells contribute to the formation of specialized ‘niches’, which are close to the marrow vasculature (‘vascular niche’) or to the endosteum (‘endosteal niche’), both of which are important in the structure and function of the bone marrow.

The term “cell” as used herein refers to the basic eukaryotic structural and functional units normally found in the body, particularly the bone marrow. Examples of cells of the vascular/hematopoietic niche and endosteal niche are listed throughout this application. The term “cell” in various embodiments may include nucleate cell and/or anucleate cells, such as mature erythrocytes or platelets. In some embodiments, the cells involved are mammalian cells. In other embodiments, they are human cells. In further embodiments, they are cells from test animals, such as mice, rats, guinea pigs, dogs, hogs, monkeys, etc.

The term “normal cell” herein refers to a cell in a state considered generally healthy or productive for its state of development, growth, activity, function, or maturity. Normal cells may also be viewed as those without an identifiable disease state or defect impairing their functions commensurate with their state of development, growth, activity, function, or maturity.

An “abnormal cell” is one which deviates from a normal state for any reason associated with a disease, trauma, or other deviation from normal development or activity.

A “modified cell” refers to a cell having a genetic complement modified by human intervention, such as through genetic modification or manipulation.

A “diseased cell” or a “diseased state cell” refers to a cell experiencing a pathologic, oncologic, or other disease challenge. Diseased cells for use in the models, designs, devices, and methods herein may be from any source, including disease cell lines or patient/donor samples. In some embodiments, such as disease models of bone marrow, both the endosteal and hematopoietic niches include diseased cells.

Diseased cells that may be included in the models described herein include leukemia [acute myeloid leukemia (AML), chronic myeloid leukemia (CML), atypical CML, chronic neutrophilic leukemia, acute lymphoblastic leukemia (ALL), etc.], multiple myeloma, smoldering myeloma, monoclonal gammopathy of undetermined significance, Non-Hodgkin lymphoma, Chronic lymphocytic leukemia (CLL), monoclonal B lymphocytosis, Hodgkin lymphoma, T-cell lymphoma, bone marrow failure syndromes, myelodysplastic syndrome (MDS), clonal hematopoiesis of indeterminate potential (CHIP), clonal cytopenias of undetermined significance (CCUS), aplastic anemia, and metastatic solid tumors that travel to the bone marrow (lung, breast, kidney, prostate, thyroid, etc.). It is understood that hemopoietic stem cells found in the endosteal microenvironment may be quiescent or proliferating.

Diseased state cells can include MSCs, macrocytes, polychromatophilic reticulocytes, aggregate reticulocytes, punctate reticulocytes, target cells, spherocytes, ovalocytes/elliptocytes, stromatocytes, sickle cells, acanthocytes, schistocytes, helmet cells, dacrocytes/teardrop cells, echinocytes/Burr cells, Pappenheimer bodies, Cabot ring cells, punctate basophilia/basophilic stippling cells, Heinz-Endrich bodies, codocytes/leptocytes, megaloblastic cells, hypochromic red blood cells, microcytic red blood cells, macrocytic red blood cells, knizocytes, degmacytes, fragmented red blood cells, Thalassemia red blood cells, degmacytes (Bite cell red blood cells), Hemoglobin C Crystal red blood cells.

The diseased state cells can also include cells of bone marrow cancers, including mature cancer cells, cancer induced angiogenesis, including, but not limited to, multiple myeloma cells and multiple myeloma precursor cells (cells exhibiting monoclonal gammopathy of unknown significance and smoldering myeloma cells), leukemic stem cells, leukemic blast cells, and leukemic promyelocytes.

A “diseased state” is an abnormal condition that negatively affects the state or function of at least part of a subject, whether or not symptoms have yet been manifested. A niche, domain, microenvironment, cell, or patient “subject to” a particular disease or malady refers to situations in which the underlying basis for a future disease state are present (such as a genetic condition, pathogen, nutrient, or biochemical deficiency, etc.), though symptoms have not yet been manifested or not yet fully manifested.

Disease states that may be studied using the models, devices, designs, and methods herein include leukemias (including Acute Myelogenous Leukemia (AML), Chronic Myelogenous or Myeloid Leukemia (CML), Atypical CML, Acute Lymphoblastic Leukemia (ALL), Chronic Lymphocytic Leukemia (CLL), Chronic Neutrophilic Leukemia, Childhood Leukemia, Chronic Myelomonocytic Leukemia, Megakaryocytic Leukemia, Chronic Myelogenous Leukemia, Juvenile Myelomonocytic Leukemia (JMML), Acute monocytic leukemia (AMoL), Atypical Chronic Myelogenous Leukemia, lymphoblastic and lymphocytic leukemias), Multiple Myeloma, Bone Marrow Failure Syndrome, clonal hematopoiesis of indeterminate potential (CHIP), clonal cytopenias of undetermined significance (CCUS), hemophagocytic lymphohistiocytosis, Wiskott-Aldrich syndrome, Bone Marrow Adiposity, aplastic anemia, Fanconi Anemia, Sickle Cell Anemia, Pure Red Cell Aplasia, myelodysplastic or myeloproliferative disorders/syndromes and neoplasms, Myelofibrosis, Paroxysmal Nocturnal Hemoglobinuria, Polycythemia Vera, Thrombocythemia, Thrombocytopenia, Thrombocytosis, and Thalassemia Major and Minor. The diseased state may also include cancers originating in bone, including osteosarcoma, chondrosarcoma, and Ewing's Sarcoma, as well as metastatic cancers including, but not limited to lymphomas (Hodgkin lymphomas, such as nodular sclerosing subtype, mixed-cellularity subtype, lymphocyte-rich subtype, or lymphocyte depleted subtype; and Non-Hodgkin, and T-cell Lymphomas), and cancers originating in other organs or tissues, including, but not limited to, the prostate (e.g. metastatic castration resistant prostate cancer), colon, breast (e.g. triple negative breast cancer), kidney (e.g. renal cell carcinoma), lung cancer (e.g. non-small cell lung cancer), and thyroid.

Diseased cells that may be included in these models include leukemia [acute myeloid leukemia (AML), chronic myeloid leukemia (CML), atypical CML, chronic neutrophilic leukemia, acute lymphoblastic leukemia (ALL), etc.], multiple myeloma, Non-Hodgkin lymphoma, Chronic lymphocytic leukemia (CLL), Hodgkin lymphoma, T-cell lymphoma, bone marrow failure syndromes, myelodysplastic syndrome (MDS), clonal hematopoiesis of indeterminate potential (CHIP), clonal cytopenias of undetermined significance (CCUS), aplastic anemia, and metastatic solid tumors that travel to the bone marrow (lung, breast, kidney, prostate, thyroid, etc.). It is understood that hemopoietic stem cells found in the hematopoietic microenvironment may be quiescent or proliferating.

Diseased state cells can include, but are not limited to, macrocytes, polychromatophilic reticulocytes, aggregate reticulocytes, punctate reticulocytes, target cells, spherocytes, ovalocytes/elliptocytes, stromatocytes, sickle cells, acanthocytes, schistocytes, helmet cells, dacrocytes/teardrop cells, echinocytes/Burr cells, Pappenheimer bodies, Cabot ring cells, punctate basophilia/basophilic stippling cells, Heinz-Endrich bodies, codocytes/leptocytes, megaloblastic cells, hypochromic red blood cells, microcytic red blood cells, macrocytic red blood cells, knizocytes, degmacytes, fragmented red blood cells, Thalassemia red blood cells, Bite cell red blood cells, Hemoglobin C Crystal red blood cells.

The diseased state cells can also include cells of bone marrow cancers, including mature cancer cells and those undergoing angiogenesis, including, but not limited to, multiple myeloma cells and multiple myeloma precursor cells (cells exhibiting monoclonal gammopathy of unknown significance and smoldering myeloma cells), leukemic stem cells, leukemic blast cells, and leukemic promyelocytes.

A system, niche, or microenvironment may also be considered as including a “diseased state” or as under disease conditions in instances where all cells present are in a normal or healthy condition for their stage of development and maturity, but other chemical, biochemical, physical, or structural aspects of the system, niche, or microenvironment include those of a diseased state or at the onset of a diseased state. Such a state can include physical factors, such as temperature, pressure, and level of hydration/fluid volume. It may also levels of particular gases (O₂, CO₂, CO, N₂, etc.), nutrients, salts, pathogens, toxins, wastes, osmotic gradients, pH, electrolytes, and buffers. A diseased state may also be characterized or mimicked by the presence or absence of biochemical factors or biochemical cues, such as growth factors, cytokines, proteins, peptides, polypeptides, adhesion molecules and peptides, nucleic acids, amino acids, biomarkers, and hematopoietic factors.

The term “microenvironment” as used herein refers to a band, block, mass, volume, layer, section, sheet, or zone of sufficient size and shape to complete one or more functions desired in a model, design, unit, and/or device described herein. In some embodiments the microenvironment includes one or more hydrogels, with or without cells. In some embodiments, the microenvironment has a vertical or thickness dimension at least as great as the diameter or greatest longitudinal dimension of the largest cell associated with the microenvironment.

The term “endosteal niche” when it refers to a microenvironment in the devices, designs, units, and models herein refers to a complex tissue without cells or with bone cells and/or mesenchymal stem cells embedded in 2- or 3-dimensions and “cemented” in a heavily calcified extracellular matrix (ECM). In some instances, the endosteal niche may be referred to as a “bone lining niche.” In native bone, the endosteal niche (“natural endosteal niche”) is the interface between the surface of the bone and the bone marrow that contains osteocytes, osteoblasts, bone matrix, and quiescent hematopoietic stem cells (HSCs), the progenitor cells for red blood cells, immune cells, and platelets. The natural endosteal niche is a complex mineralized tissue with bone cells embedded in a three-dimensional, heavily calcified extracellular matrix (ECM). In some embodiments herein, the endosteal niche units or layers are mineralized. In some embodiments, the endosteal niche may contain cells or other components normally found in cortical endosteum (endosteal lining of cortical bone), osteonal endosteum (endosteal lining of the osteonal canals), and/or trabecular endosteum (endosteum coating interior surfaces of trabeculae).

In some embodiments, the mineralized microenvironment/endosteal niche may be formed using fragmented mineralized collagen or bone, including for instance shards of bone.

The endosteal niche can be recreated via encapsulating or seeding cells including, but not limited to, healthy, diseased or modified osteoblasts, osteoclasts, osteocytes, MSCs, osteoprogenitors, or without cells. It can be made of or include other endosteal elements or bone substitutes (such as calcium and/or phosphate ceramics) or via other mineralization methods (such as via osteoinductive medium).

The implementation of this niche can be achieved via layer by layer methods, using different geometries and method of synthesis including but not limited to ring, disk, drops, spheres, shards, centrifugation of high-density collagen followed by mineralization, or any combination thereof.

“Fibrillogenesis” refers to the development of fine fibrils normally present in collagen fibers. Collagen cross-linking in native collagen contributes to fibrillogenesis, matrix stability, and elasticity.

“Type I collagen” or “Type 1 collagen” refers to the fibrillar-type collagen that is the most abundant form of human collagen and the key structural composition of several tissues.

The term “hydrogel” as used herein refers to a gel including a cross-linked network of water-soluble polymers capable of forming a matrix mimicking a natural extracellular matrix and supporting the biological materials and activities of interest to the present studies. Commercially available hydrogels include the MATRIGEL® matrix (available from Corning Inc., Tewksbury, Mass.); poly[2-(methacryloyloxy)ethyl dimethyl(3-sulfopropyl)ammonium] (PMEDSAH) hydrogels or copolymers or blends thereof; glycoprotein hydrogels, such as fibronectin hydrogels and laminin hydrogels; protein hydrogels, such as those derived from collagen, albumin, fibrin, or silk proteins; polysaccharide hydrogels, such as those derived from glucan, hyaluronic acid, chitosan, agarose, and alginate; synthetic hydrogels composed of synthetic monomers such as those selected from the group of poly(ethylene glycol) (PEG), poly(vinyl alcohol) (PVA), poly(ethylene oxide) (PEO), poly(acrylic acid) (PAA), poly(hydroxyethyl methacrylate) (PHEMA), poly(methacrylic acid) (PMMA), polypropylene fumarate-co-ethylene glycol (P(PF-cop-EG)), poly(acrylamide) (PAAm), and poly N-isopropylacrylamide (PNIPAAm); and hybrid synthetic-biologic hydrogels having combined monomers of synthetic and biological materials, such as PEG-peptide hydrogels, including PEG-fibrinogen hydrogels. The term “hydrogel”, as used herein, is understood to include a single type of hydrogel material, such as one of the individually listed hydrogel materials above, or a mixture or combination of two or more individual hydrogel materials, such as a combination of the MATRIGEL® matrix with collagen and/or a fibronectin hydrogel.

In some embodiments, the hydrogel matrix includes collagen fibers. In some embodiments, the collagen fibers are selected from the group of collagen type I (also known as Type 1 collagen), collagen type II, collagen type III, collagen type IV, collagen type V, collagen type VI, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, and collagen type XX, or a combination thereof.

A “cross-linking agent” herein refers to an agent that facilitates the cross-linking of polymer chains to form a matrix of cross-linked polymer chains, such as a collagen or hydrogel. For various matrices, the cross-linking agent will vary by the polymer chains involved. Polyvinyl alcohol hydrogels may be cross-linked using sodium borate/boric acid as a cross-linking agent. Glyoxal may be used as a cross-linking agent for polyvinyl alcohol, starch, cellulose, or protein and gelatin hydrogels. Other hydrogel/cross-linking agent combinations include: polyethylene hydrogel/silane, agarose and chitosan hydrogels/oxidized dextrins, chitosan/glutaraldehyde, guar gum hydrogels/epichlorhydrin, Gellan gum hydrogels/endogen polyamine spermidine, glycol chitosan hydrogels/oxidized alginate, hydroxamated alginates/zinc, alginate beads/zinc, scleroglucan/Borax, poly(acrylic-co-vinylsulfonic) acid hydrogels/ethylene glycol dimethacrylate (EDGMA), polyacrylamide hydrogels/N,N′-methylenebisacrylamide, and polyacrylamide/guar gum graft copolymer hydrogels/glutaraldehyde. A “cross-linking treatment” herein refers to any method of subjecting a group of non-linked polymers to an agent, force, or set of conditions that facilitate cross-linking of the polymers to form a desired matrix. Examples of cross-linking treatments include regimens of photochemical cross-linking or radiation-induced cross-linking.

In some embodiments herein, the matrix includes one or more acidic polymers selected from the group of polyacrylic acid, polymethacrylic acid, sulfonated polymer, phosphorylated proteins or peptides, phosphorylated synthetic polymers, sulfated polysaccharides, sulfated glycoproteins, polyaspartic acid, polyglutamic acid, polyaspartate, polyvinyl phosphate, and polyvinyl phosphonate, or combinations thereof.

Calcium-containing ionic materials that can be used as calcium “drug” or as the calcium source in a “mineralizing solution” are calcium chloride (anhydrous: CaCl₂, monohydrate: CaCl₂.H₂O, dihydrate: CaCl₂.2H₂O, or hexahydrate: CaCl₂.6H₂O), dicalcium phosphate dehydrate (CaHPO₄.2H₂O; DCPD), calcium sulphate dehydrate (CaSO₄.2H₂O; CSD), calcium sulphate hemihydrate (CaSO₄.½H₂O; CSH), calcium sulphate (CaSO₄), calcium acetate (anhydrous: Ca(C₂H₃O₂)₂, monohydrate: Ca(C₂H₃O₂).2H₂O, or dihydrate Ca(C₂H₃O₂)₂.2H₂O), calcium citrate (Ca₃(C₆HsO₇).4H₂O), calcium fumarate (CaC₄H₂O₄.3H₂O), calcium glycerophosphate (CaC₃H₅(OH₂)PO₄), calcium lactate (Ca(C₃HsO₃)₂.5H₂O), calcium malate (dl-malate: CaC₄H₄O₅.3H₂O, 1-malate: CaC₄H₄O₅.2H₂O, or malate dihydrogen: Ca(HC₄H₄O₅)₂.6H₂O), calcium maleate (CaC₄H₂O₄.H₂O), calcium malonate (CaC₃H₂O₄.4H₂O), calcium oxalate (CaC₂O₄), calcium oxalate hydrate (CaC₂O₄.H₂O), calcium salicylate, (Ca(C₇H₅O₃)₂.2H₂O), calcium succinate (CaC₄H₆O₄.3H₂O), calcium tartrate (d-tartrate: CaC₄H₄O₆.4H₂O; dl-tartrate: CaC₄H₄O₆.4H₂O; mesotartrate: CaC₄H₄O₆.3H₂O), and calcium valerate (Ca(C₅H₉O₂)₂).

Phosphate-containing ionic materials that can be used as a phosphate source in a “mineralizing solution” include dicalcium phosphate dehydrate (DCPD), sodium phosphate (Na₂HPO₄, NaH₂PO₄ or a mixture thereof; non-hydrated or hydrated species like Na₂HPO₄.2H₂O, Na₂HPO₄.7H₂O, Na₂HPO₄.12H₂O, NaH₂PO₄.H₂O, NaH₂PO₄.2H₂O), calcium glycerophosphate (CaC₃H₅(OH₂)PO₄), potassium orthophosphate (K₃PO₄), dihydrogen potassium orthophosphate (KH₂PO₄), monohydrogen potassium orthophosphate (K₂HPO₄), and sodium orthophosphate (Na3PO₄.0H₂O and Na₃PO₄.12H₂O).

The term “immature cells” or “immature bone cells” used herein in refers to any cell type that is naturally found in a hematopoietic/vascular/stem cell niche or in an endosteal niche and has not reached full maturation, including those at a primary or intermediate level of maturation. Examples include mesenchymal stem cells, hematopoietic stem cells, osteoblasts, and progenitor cells, multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, chondrocyte precursor cells, mesenchymal stem and progenitor cells or mesenchymal stromal progenitor cells (MSPCs).

These immature cells also include those undergoing developmental pathways in hematopoietic, mesenchymal, bone and vascular lineages including but not limited to, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), hemopoiesis, leukopoiesis, erythropoiesis, granulopoiesis, lymphopoiesis, etc. Specific cell examples include, but are not limited to, reticulocytes, hemocytoblasts, proerythroblasts, erythroblasts, normoblasts, polychromatic erythroblasts, myeloblasts, progranulocytes, lymphoblasts, monoblasts, promonocytes, monocytes, megakaryoblasts, megakaryocytes, megakaryocyte progenitor cells, erythrocyte progenitor cells, megakaryocyte-erythrocyte progenitor cells, pro-natural killer cells, pro-B cells, pre-B cells, common myeloid progenitor cells, common lymphoid progenitor cells, myeloid stem cells, myeloblasts, promyelocytes, myelocytes, basophilic myelocytes, basophilic meta-myelocytes, metamyelocytes, band forms, eosinophilic myelocytes, eosinophilic meta-myelocytes, neutrophilic myelocytes, neutrophilic meta-myelocytes, and neutrophilic band cells.

In some embodiments, the final matrix further includes microvascular fragments. The term “microvascular fragments” refers to fragments of adipose microvasculature generally collected and chopped to a fine size, followed by digestion with collagenase, usually with agitation, followed by centrifugation and separation using a series of filters of defined pore size. In some examples, larger pieces may be removed using a 200 μm nylon filter and individual cells may be removed using a 20 μm filter membrane. The microvascular fragments are also known as “processed microvascular tissue” or “adipose tissue-derived microvascular fragments (ad-MVF).”

In some embodiments, the final mineralized matrix includes mesenchymal stem cells and microvascular fragments.

The term “media” herein refers to is any fluid, such as conventional cell culture medias, that contain nutrition, oxygen or other gases, or other elements needed for the maintenance, growth, or testing of cells or agents in one or both of the endosteal and vascular microenvironments of the designs and devices herein. In some embodiments, the media includes a fluid capable of delivering other biological molecules to the microenvironments, including, but not limited to, growth factors, chemokines, cytokines, hormones, etc. In some embodiments, the media includes one or more drugs or other biologically or medically active compounds to be tested for effects in bone marrow and/or the cells of the specific design or device. In some embodiments, the media used is designed to mimic metastasis or a metastatic condition. In other embodiments, the media used is designed to mimic pre-metastasis or a pre-metastatic condition.

The term “osteoprogenitor” or “osteoprogenitor cell” refers to the stem cells of bone and form osteoblasts. Osteoprogenitor cells form a population of stem cells capable of differentiation into more specialized bone-forming cells, i.e. osteoblasts and osteocytes. In some embodiments, the osteoprogenitor cells are present as stem cells. In other embodiments, the osteoprogenitor cells may be in forms naturally present in mature bone, i.e., they may be present as flattened spindle cells that are also known as “inactive osteoblasts.”

The terms “vascular niche” or “hematopoietic niche” refer to a segment or portion of materials in the present designs that act in the biological fashion of or mimic the segments of natural bone marrow close to or permeated by marrow vasculature and stroma. In the units and models herein, the “vascular niche” refers to a soft matrix loaded with hematopoietic and mesenchymal stem cells. In exemplary embodiments, the vascular niche is close to a fluidic channel, with or without endothelial cells.

Current in vitro models (a) lack the vascular or endosteal niches entirely; (b) are based on culture of stem cells in monolayers on calcium and phosphate scaffolds; or, in vivo (c) rely on time-consuming (8-12 wk) xenograft models in vivo.

A “microfluidics device” is a device designed and manufactured at a scale to complete processes, such as fluid flow, of volumes of fluid in very small volumes, such as, microliter, nanoliter, or picoliter volumes.

A “fluidic(s) device” or “microfluidic(s) device” is a device designed and manufactured at a scale to complete processes, such as fluid flow, of volumes of fluid in very small volumes, such as milliliter, microliter, nanoliter, or picoliter volumes.

The term “organ-on-a-chip” refers to a microdevice, such as a fluidics or microfluidics device, that mimics or recapitulates the microarchitecture and function of a living organ or a subpart thereof. Such devices provide an alternative to animal testing and allow for tests directed at specific biological activities or combinations thereof. In a bone-on-a-chip model, the microdevice allows study of the effects of set conditions or agents on one or more functions of natural bone marrow components.

The term “osteoprogenitor” herein refers to any of the bone or mesenchymal precursor cells committed to the bone lineage and capable of producing osteoblasts and osteocytes. Osteoprogenitor cells may also be referred to as preosteoblasts or preosteocytes. The term “osteochondroprogenitor” refers to progenitor cells arising from mesenchymal stem cells in the bone marrow that may differentiate into osteoblasts or chondrocytes upon exposure to signaling molecules.

The term “shard” herein refers to irregular fragments formed from elements of the endosteal niche. In some embodiments, the shards include osteoblasts. Optionally, these are formed by breaking apart a different structure, such as a sphere or a disk or a drop. The term includes bone-like fragments that are made by pre-mineralization or post-mineralization formation (with regard to when cells are added).

The term “sphere” herein is understood to include materials formed into a generally rounded, solid material, such as a hydrogel, optionally containing biological aspects of the endosteal and/or vascular niches described herein. While the term “sphere” is understood to include spherical bodies wherein the surface is equidistant from the center, embodiments include such bodies that are not equally or uniformly shaped or sized. As examples, such bodies may be generally rounded, lobular, rod-shaped, cylindrical, bulbous, oblong, oval, ovoid, elliptical, ellipsoid, spherical, and spheroidal (including oblate and prolate spheroidal forms). In other embodiments, the “spheres” may be replaced with microenvironments that are of any shape defining a form, including those having squared, rectangular, or trapezoidal surfaces, or pyramidal, conical, polygonal, polyhedronal, columnar, rounded rectangular, rounded cubic, irregular, amorphous shapes, optionally with convex or concave surfaces, or other shape, including amorphous or amorphic shapes, or combinations thereof, in total or in cross-section, that allow the functions described herein. In general, the term sphere is used herein to refer to a pre-mineralization (or un-mineralized) hydrogel body, in contrast to a shard.

The term “substantial contact” used herein in reference to two three-dimensional layers or sheets associated with a device or model herein refers to an arrangement that allows a desired amount of materials to pass between the sheets or layers in question. In some embodiments, the substantial contact is in situations wherein at least ten percent of the surface area of one three-dimensional layer or sheet is in contact with at least ten percent of the surface area of another three-dimensional layer or sheet. In some embodiments, the substantial contact involves direct contact by greater than one quarter of the surface area of the first and second three-dimensional layers or sheets in question. In other embodiments, the substantial contact includes contact between at least one third of the surface area of the first and second three-dimensional layers or sheets in question.

The terms “substantially” and “substantially all” are intended to mean greater than 90% of the matter, definition, or relationship indicated (for instance 90% to 100%). Typically, the element is at least 95% of the matter, definition or relationship, more typically at least 99% of the matter, definition, or relationship indicated.

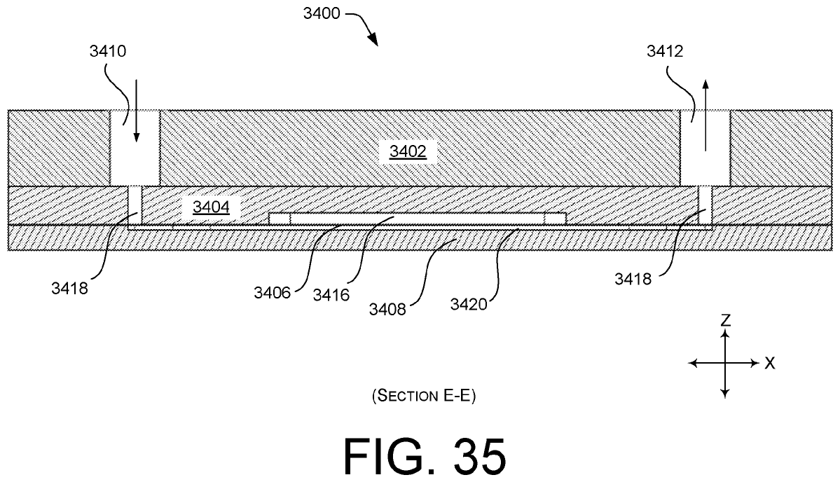

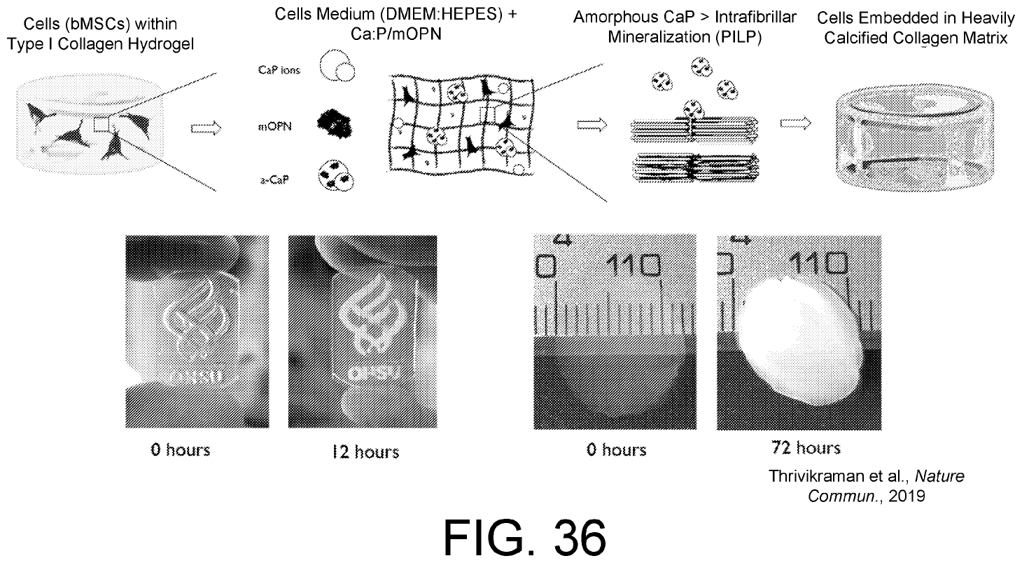

In some embodiments, an endosteal niche for use in the designs and methods herein may be prepared using the protocol provided in Example 1. FIG. 35 illustrates the formation and mineralization of a cell-laden hydrogel of the type useful in designs and methods herein. See also Thrivikraman et al. (Nature Comm., 10(1):3250, 2019) and WO 2020/069033, which provide both scanning electron and transmission electron micrographs (SEM and TEM) of the ultrastructural similarity of native bone and the mineralized hydrogel material useful in the herein provided models, designs, and methods. Thrivikraman et al. (Nature Comm., 10(1):3250, 2019) and WO 2020/069033 showed the chemical similarity of the mineralized hydrogel to native bone in FTIR spectral, mineral to matrix ratio, and crystallinity index aspects. Mineralized samples had comparable values to that of native bone, and both were significantly higher than non-mineralized controls (****p<0.0001 ANOVA/Tukey).

Elements of the endosteal niche (such as the mineralized collagen) mentioned above can be cut, broken, or mechanically agitated to form “shards”. Shards may or may not encapsulate cells. Shards can be associated with the bone marrow model in a variety of ways, for instance they may be embedded throughout the cell laden hydrogel (for instance, in the vascular niche).

Formation of Endosteal Shards Seeded with Human Fetal Osteoblasts (hfOB)

Shards are created by mechanical dissociation of mineralized collagen/cell endosteal niche. These shards allow for elements of the niche to be applied throughout stem cell laden matrix. They may be created to varying sizes depending upon channel width or other needs of the experiments.

After formation of the mineralized niche outlined herein, mineralized disks may be removed from their wells and placed into a 35 mm tissue culture dish containing enough DMEM-HEPES complete media to embed but not cover the disks (about 3-5 ml). Using a clean rounded scalpel, the disks are then chopped into roughly 0.5-1 mm² pieces. The media and mineralized pieces are then passed through gradually smaller needles (18 g, 23 g, 25 g) with a 5 ml syringe to create the final shard morphology. Shards are pipetted into a microcentrifuge tube and spun at 300×g for 3 min. Used media is removed and replaced with fresh DMEM+ HEPES complete. Shards+media are transferred to a tissue culture well and placed into a 37° C. incubator with humidified atmosphere containing 5% CO₂.

Elements of the endosteal niche, for instance mineralized collagen may also be formed into spheres. These spheres may or may not include cells. The spheres can be embedded throughout the cell laden hydrogel (for instance, the vascular niche), or as its own separate niche. The spheres (or droplets) can be made of collagen or other types of hydrogel in the appropriate media. They may or may not include cells. They may be synthesized via other methods (such as using microfluidics). Spherical or similarly shaped microenvironments may be formed using a microfluidic droplet generator device or an O-W emulsion method.

To create two separate regions (niches) in one compartment, the cells for endosteal niche are encapsulated in a hydrogel droplet via emulsion, mineralized and introduced to the device. In one embodiment the droplets may include cells, in another embodiment the cells can be seeded on the hydrogel droplet (before or after mineralization). These droplets will be mineralized by aforementioned method to create the endosteal niche. After mineralization, the endosteal niche will can be embedded in the vascular niche. In one embodiment vascular niche is a premixed hydrogel with stem cells. This combination is then cross-linked by applying suitable crosslinking conditions.

Example 3 provides a representative protocol for formatting mineralized hydrogel (exemplified using rate tail collagen 1) drops/beads/spheres, and loading them with live cells (exemplified with human fetal osteoblasts).

Vascular Niche

Structurally, the vascular niche is a soft matrix loaded with quiescent hematopoietic and mesenchymal stem cells permeated by vasculature and stroma. The vascular niche can be recreated via a variety of compositions such as elements of the hematopoietic niche, including but not limited to healthy, diseased, or modified MSCs, HSCs, Progenitor cells, endothelial cells, pericytes, neurons, encapsulated or seeded in 2D or in 3D hydrogels including but not limited to MATRIGEL®, collagen, fibrin, fibronectin, or any combinations thereof. In one embodiment, human MSCs and HSCs are encapsulated in MATRIGEL® to recreate the vascular niche of the marrow.

An example vascular niche may include human bone marrow derived normal mesenchymal stem cells (hMSCs) and human primary Bone Marrow CD34+ Cells (HSCs) seeded into MATRIGEL® basement membrane. hMSC Cells are used from passages 2-6, having been cultured in Mesenchymal Stem Cell Basal Medium for Adipose, Umbilical and Bone Marrow-derived MSCs (ATCC PCS-500-030) supplemented with Mesenchymal Stem Cell Growth Kit for Bone Marrow-derived MSCs (ATCC PCS-500-041) containing rh FGF basic: 125 pg/mL, rh IGF-1: 15 ng/mL, Fetal Bovine Serum: 7%, L-Alanyl-L-Glutamine: 2.4 mM and 1% antibiotic solution. Cells are dissociated using TrypLE express (Gibco) and centrifuged at 300×g for 3 minutes. Cells are then suspended in growth media at desired concentrations.

HSCs are stored in liquid N₂ until day of experiments and are thawed, suspended in Hanks Balanced Salt Solution at 1:10 ratio, spun at 300×g or 3 minutes after which media is carefully drawn off and the cell pellet is suspended in the appropriate amount of MSC growth media. Prior to cell dissociation (hMSC) and thawing (HSC), Matrigel is allowed thaw. Cells are mixed with MATRIGEL® in a 1:1 cell containing solution to MATRIGEL® mixture. At this point the cell, MATRIGEL® mixture may also be seeded with human osteoblast-containing mineralized shards, or mineralized collagen spheres. Further, an entire disk of mineralized collagen containing human osteoblasts may be laid into a channel of the microfluidic devise prior to this point. The seeded MATRIGEL® mixture is pipetted into the designated channel of the microfluidic device. The seeded microfluidic devise is placed into a humid 37° C. incubator with a 5% CO₂ atmosphere and the cell seeded MATRIGEL® is allowed to gel for a minimum of 30 minutes after which the device is removed and the appropriate growth media is fed through adjoining channels.

Alternative cytokine mixtures may be used, including media which contains 20 ng/ml EPO and 100 ng/ml TPO but no aprotinin, G-CSF, SCF or EGM-2 Bullet kit reagent; and others which contain half the previous volumes of EPO and TPO but are otherwise cytokine free, see Table 3. The entire device, media and pumping apparatus is placed into the incubator for the entire length of the experiment. The devices (for instance, static or microfluidic devices) are periodically removed for inspection of channel(s) containing cells and/or media. Media may be replaced depending upon the condition of the experiment to solution containing growth factors, drugs, nutrients, or other materials.

The entire device, media and pumping apparatus is placed into the incubator for the entire length of the experiment. The microfluid devices are periodically removed for inspection of channel(s) containing cells and/or media. Media may be replaced depending upon the condition of the experiment to solution containing growth factors, drugs or other.

High-Throughput Well Plate System

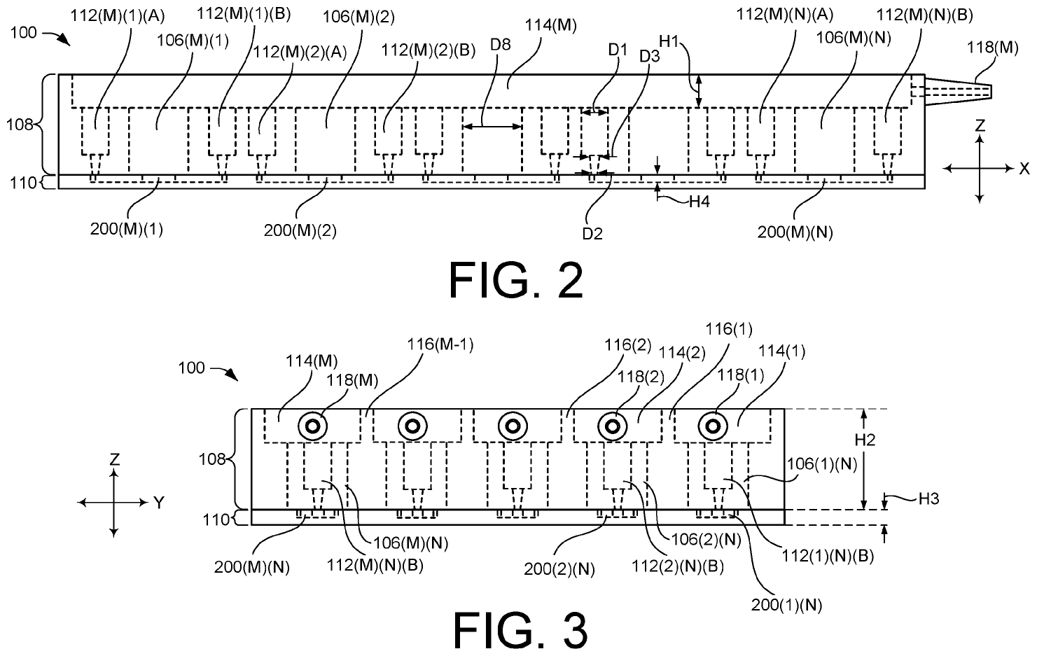

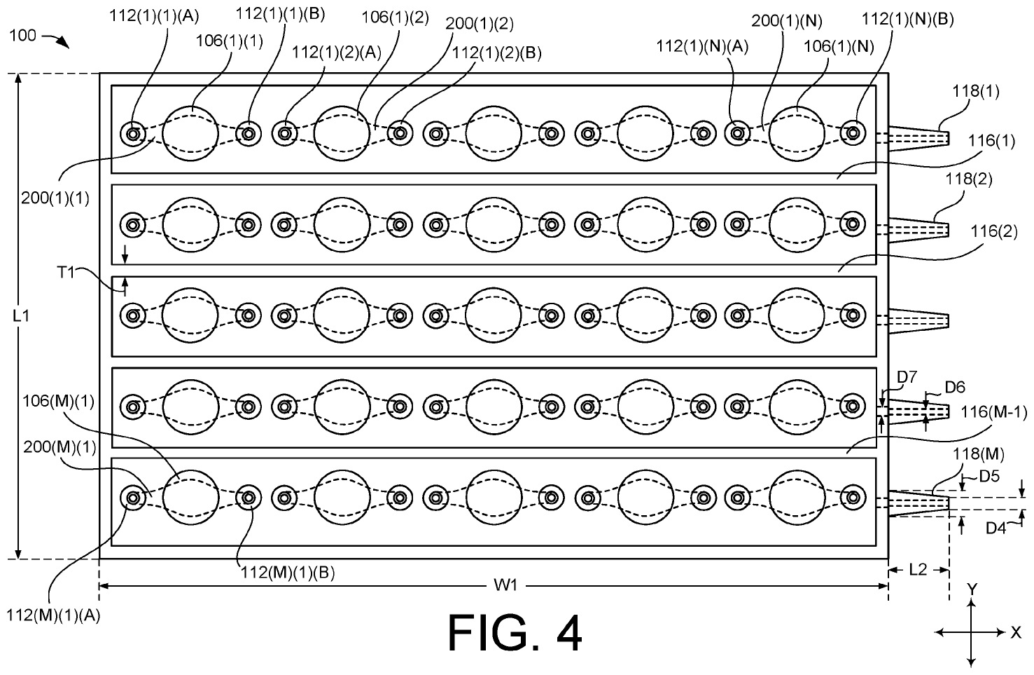

Referring to FIG. 1, there is illustrated an example well plate 100, according to an example embodiment. The well plate 100 is shown from a perspective view in FIG. 1. FIG. 2 illustrates a front view of the example well plate 100 of FIG. 1, FIG. 3 illustrates a side view of the example well plate 100 of FIG. 1, and FIG. 4 illustrates a top view of the example well plate 100 of FIG. 1. The well plate 100 may constitute a system that is configured to culture cells, such as bone marrow cells. Additionally, or alternatively, the well plate 100 may be part of a larger system with additional component parts, as will be described in more detail below.

The well plate 100 has a top 102 and a bottom 104, as well as four sides. Accordingly, the well plate is generally cuboidal. It is to be appreciated, however, that the well plate 100 disclosed herein is not limited to being cuboidal, as the well plate 100 may be of any polygonal shape, such as cylindrical- or disk-shaped with a single continuous side surface between the top 102 and bottom 104, triangular-shaped with three sides between the top 102 and bottom 104, pentagonal-shaped with five sides between the top 102 and the bottom 104, and so on.

As shown in FIG. 1, the well plate 100 may include an M×N array of media wells 106, with M rows of media wells 106 and N columns of media wells 106, where M can be any suitable integer and N can be any suitable integer. Accordingly, the first row of media wells 106 includes media wells 106(1)(1) through 106(1)(N). A second row of media wells 106 includes media wells 106(2)(1) through 106(2)(N), and so on and so forth until an M^{th} row of media wells 106 that includes media wells 106(M)(1) through 106(M)(N). The example M×N array of media wells 106 shown in FIG. 1 is a 5×5 array of media wells 106, where M=5 and N=5. This configuration enables high-throughput, in vitro culturing of three-dimensional (3D) human organ models, such as bone marrow models, in small volumes and for several applications, such as drug toxicity and drug efficacy. This high-throughput system can allow for performing a relatively large amount of experiments, as compared to existing systems, and it allows for doing so in a robust and reliable manner. In the pharmaceutical industry, this allows for realizing the full potential of the in vitro organ models in a drug discovery pipeline.

An individual media well 106 is configured to receive fluid media. For example, each media well 106 may be filled with a cell culture media, such as media that contains nutrition, oxygen or other gases, or other elements for the maintenance, growth, or testing of cells or agents (e.g., cells in hydrogel), for instance testing of drug candidate compounds or other compounds of interest; more generally, the devices can be used to characterize any perturbation. Because the media wells 106 are isolated from one another, the media wells 106 may be filled with different types of media in the same well plate 100. For example, a first media well 106(1)(1) may be filled with a first type of media, a second media well 106(1)(2) may be filled with a second type of media, and so on and so forth. Similarly, each well can have different compound (e.g., drug or drug candidate) variants, concentrations, combinations, and so forth. In this configuration, there is no possibility of cross contamination due to the isolated nature of each media well 106. The media well 106 is shown as a cylindrical media well, but any suitable shape can be utilized for the media well 106.

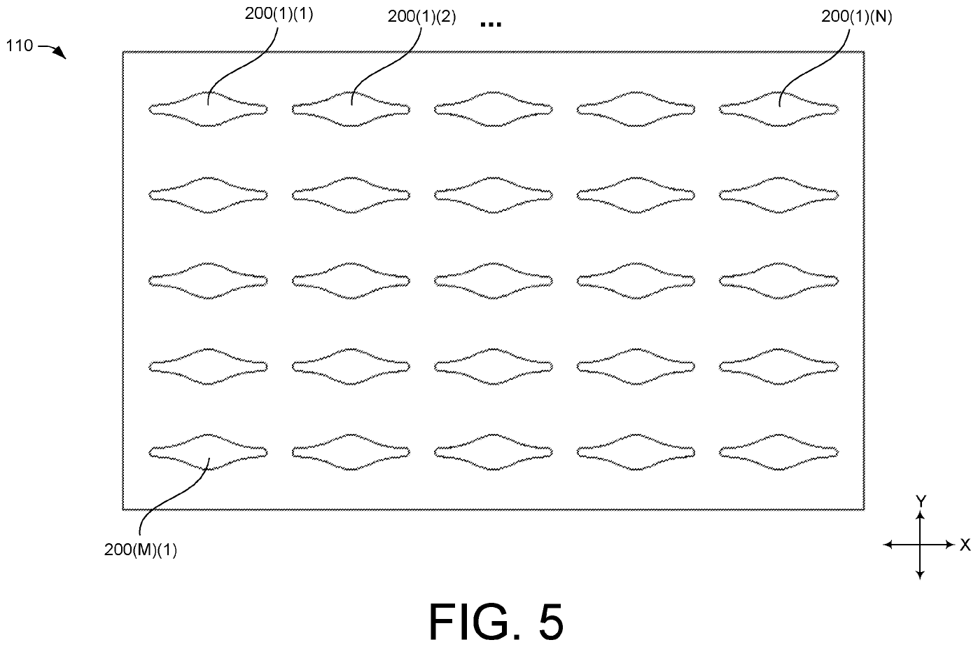

FIG. 1 depicts an assembly of the well plate 100 where a first substrate 108 that includes the media wells 106 is stacked atop a second substrate 110 that includes an M×N array of hydrogel chambers 200. Because the array of hydrogel chambers 200 is also an M×N array, there is a one-to-one correspondence between a media well 106 and a hydrogel chamber 200, and each hydrogel chamber 200 may be positioned underneath its corresponding media well 106. As depicted in FIG. 5, prior to assembly of the well plate 100 shown in FIG. 1, the second substrate 110 may have an array of recessed areas in a top of the second substrate 110. These recessed areas in the top of the second substrate 110 form the hydrogel chambers 200 when the top of the second substrate 110 is affixed to the bottom of the first substrate 108. In addition, prior to the assembly of the well plate 100 shown in FIG. 1, the first substrate 110 may have an array of through-holes. These through-holes are capped with a permeable barrier at a bottom end, and they form the media wells 106 after the top of the second substrate 110 is affixed to the bottom of the first substrate 108. In embodiments without the permeable barrier (membrane), the well media is in full contact with the hydrogel layer. Although the well plate 100 is shown as being made of multiple substrates, it is to be appreciated, that the well plate 100 can be a single monolithic piece of material.

The well plate 100 may be made of any suitable material, combination of materials, or composite materials. For example, the well plate 100 can be made of any of the polymer/plastic materials described herein, such as a laboratory-grade plastic including, without limitation, polycarbonate plastic, polystyrene, polyethylene, Polyethylene terephthalate (PET), and the like. It is to be appreciated that the well plate 100 can be made of any other suitable rigid or semi-rigid material that is suitable for cell culturing.

In some embodiments, the well plate 100 may be manufactured using an injection molding technique, or an extrusion technique, the processes for which are apparent to a person having ordinary skill in the art. Other manufacturing techniques that may be used to manufacture the well plate 100 include machining a material into the shape of the well plate 100, or into component parts (e.g., the substrates 108 and 110) of the well plate 100 that are attached together during manufacture using any suitable fastening means, such as screws, pins, joints, adhesives, or the like. Any other subtractive manufacturing techniques can be used besides machining. Additionally, additive manufacturing techniques, such as 3D printing, can be used to manufacture the well plate 100.

As shown in FIG. 1, the well plate 100 may further include an array of loading ports 112. As depicted in FIG. 1, the loading ports 112 may outnumber the media wells 106 in that there may be more loading ports 112 than there are media wells 106 in a given well plate 100. In other words, a first number of the array of loading ports 112 may be greater than a second number of the array of media wells 106. In this configuration, a pair of loading ports 112 may be positioned on opposing sides of a given media well 106. For example, a first loading port 112(1)(1)(A) may be positioned adjacent to a first media well 106(1)(1) and a second loading port 112(1)(1)(B) may be positioned adjacent to the first media well 106(1)(1). In some embodiments, the loading ports 112(1)(1)(A) and 112(1)(1)(B) are positioned beside the media well 106(1)(1) (e.g., within a common substrate 108 at generally the same level). It is to be appreciated, however, that each media well 106 may be associated with a single loading port 112 instead of multiple loading ports 112. It is also to be appreciated that the loading ports 112 may be defined elsewhere in the well plate 100 (e.g., elsewhere in either substrate 108 or 110) such that the loading ports 112 need not be positioned beside their corresponding media well 106 or even adjacent thereto. A loading port 112 may nevertheless be associated with a media well 106 by virtue of being in fluid communication with the hydrogel chamber 200 that underlies the media well 106.

For instance, as illustrated in FIG. 2, an individual loading port 112 is shown as being in fluid communication with an individual hydrogel chamber 200. “In fluid communication,” as used herein can mean that a conduit or an opening connects two chambers such that fluid may flow from one chamber to another via the conduit/opening. Accordingly, the loading port 112 may direct any fluid, such as a hydrogel containing cells, into the hydrogel chamber 200, and the hydrogel chamber 200 may receive the fluid (e.g., a hydrogel containing cells) via the loading port 112 in which the hydrogel chamber 200 is in fluid communication. In the example well plate 100, the hydrogel chamber 200 is positioned underneath a corresponding loading port(s) 112. For example, the hydrogel chamber 200(M)(1) is shown as being positioned underneath the loading port 112(M)(1)(A), as well as underneath the loading port 112(M)(1)(B).

An individual loading port 112 may also be configured to allow a hydrogel containing cells to egress from the hydrogel chamber 200. In this sense, a loading port 112 may also be considered an “unloading” port for purposes of unloading or emptying the hydrogel chamber 200. In some embodiments, loading of the hydrogel chamber 200 may be accomplished by expressing/injecting/dispensing hydrogel from a pipette, a syringe, an automated liquid handling system or bioprinter, or a similar tool into the loading port 112, which, in turn, directs the hydrogel into the hydrogel chamber 200. In some embodiments, unloading may be accomplished by inserting the outlet of an automated liquid handling system or bioprinter, or a pipette, a syringe, or a similar tool into a loading port 112 and sucking out the hydrogel from the hydrogel chamber 200 (e.g., by pulling on a plunger of the pipette, syringe, etc., or reversing the flow of a liquid handling system). It will be understood that such devices can also be used to load, remove, or otherwise move media within or around the device.

In some embodiments, an individual loading port 112 has a first end having a first inner diameter, D1, which is configured to receive a pipette tip or any needle tip or needle-like device. The loading port 112 may also have a second end having a second inner diameter, D2, less than the first inner diameter, D1. This second end with the smaller inner diameter, D2, may be positioned at, or next to, an opening to the hydrogel chamber 200. This shape of the loading port 112 may accommodate a pipette tip for easy loading and/or unloading of the hydrogel chamber 200 with hydrogel containing cells. In some embodiments, a top portion of the loading port 112 may have an inner diameter, D1, for instance a constant diameter of about 4 mm, and a bottom portion of the loading port 112 may have a progressively narrowing diameter that narrows progressively in a direction towards the opening to the hydrogel chamber 200. Specifically contemplated is a top portion of the loading port 112 with a variable (for instance, tapered) inner diameter, D1, or with a constant inner diameter, D1. Accordingly, the bottom portion of the loading port 112 may narrow from a third inner diameter, D3, of about 1.5 mm to the second end having an inner diameter, D2, of about 1 mm. In some embodiments, the loading port comprises a Luer connector for locking a tip of a pipette and/or a syringe into the loading port 112 during loading/unloading.

Turning to FIG. 5, there is illustrated a top view of the second (e.g., bottom) substrate 110 of the example well plate 100 of FIG. 1. As shown in FIG. 5, the second substrate 110 includes an M×N array of recessed areas that form hydrogel chambers 200 when the well plate 100 is assembled (e.g., when a top of the second substrate 110 is affixed to a bottom of the first substrate 108). The recessed areas in the second substrate 110 that ultimately form the hydrogel chambers 200 in the assembled well plate 100 may be of any suitable shape, such as a modified elliptical shape depicted in FIG. 5. For instance, the assembled well plate 100 may be of other shapes such as rectangular contours, spirals, and so forth. An inner area of the hydrogel chamber 200 may be generally circular in order to correspond to the cylindrical shape of the media well 106 that is positioned above the hydrogel chamber 200 in the assembled well plate 100. Other shapes of hydrogel chamber 200 include a deformed elliptical shape, a spiral, and shapes that reduce or minimize the likelihood of collapse. To accommodate a point of ingress and/or egress to/from the hydrogel chamber 200, the hydrogel chamber 200 may include features that extend laterally from the generally circular inner area of the chamber to form the shape depicted in FIG. 5.

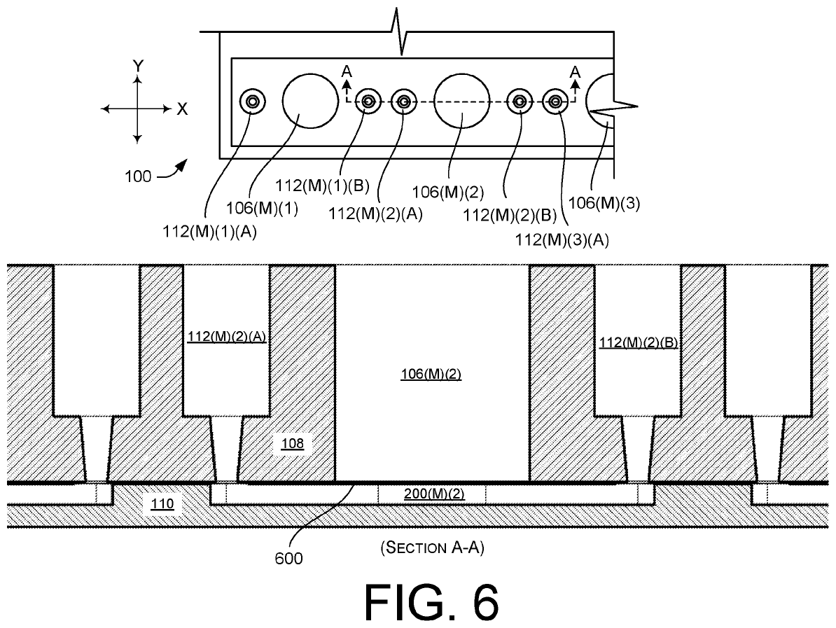

Turning to FIG. 6, there is illustrated a cross-sectional view of a portion of the example well plate 100 of FIG. 1, taken along section line A-A. This portion depicts a zoomed-in view of a media well 106(M)(2) that is positioned above a corresponding hydrogel chamber 200(M)(2), as well as a pair of loading ports 112 including a first loading port 112(M)(2)(A) and a second loading port 112(M)(2)(B). Each loading port 112(M)(2) is positioned beside the media well 106(M)(2), and is in fluid communication with the hydrogel chamber 200(M)(2). As further depicted in FIG. 6, a permeable barrier 600 (sometimes referred to herein as a “permeable membrane 600,” a “porous membrane 600,” or “permeable layer 600”) is interposed between the media well 106(M)(2) and the hydrogel chamber 200(M)(2). Accordingly, the media well 106(M)(2) may include a bottom end that is covered with the permeable barrier 600. For example, the permeable barrier 600 may be affixed (e.g., bonded) to the bottom end of the media well 106(M)(2). In some embodiments, the permeable barrier 600 may include PDMS or polycarbonate (PC) or polyethylene terephthalate (PETE) capable of allowing passage of desired media and contents, such as nutrients, metabolites, waste, etc. One commercially available membrane for this purpose is the PE1009030 10.0 micron, 90 mm polyester (PETE) membrane filters available from Sterlitech Corporation (Kent, Wash., USA). In some embodiments, the permeable barrier 600 may be greater than the cross-sectional area of the media well 106(M)(2), such as slightly greater than to cover the bottom end of the media well 106(M)(2) without waste of material. In some embodiments, the permeable barrier 600 can span a larger area (with through holes for access into the hydrogel layer), such as an entirety of the top end of the hydrogel chamber 200(M)(2), and/or an entirety of the area of the first substrate 108 or the second substrate 110. As depicted in FIG. 6, the hydrogel chamber 200(M)(2) is positioned underneath the permeable barrier 600, and the media well 106(M)(2) is positioned atop the permeable barrier 600.

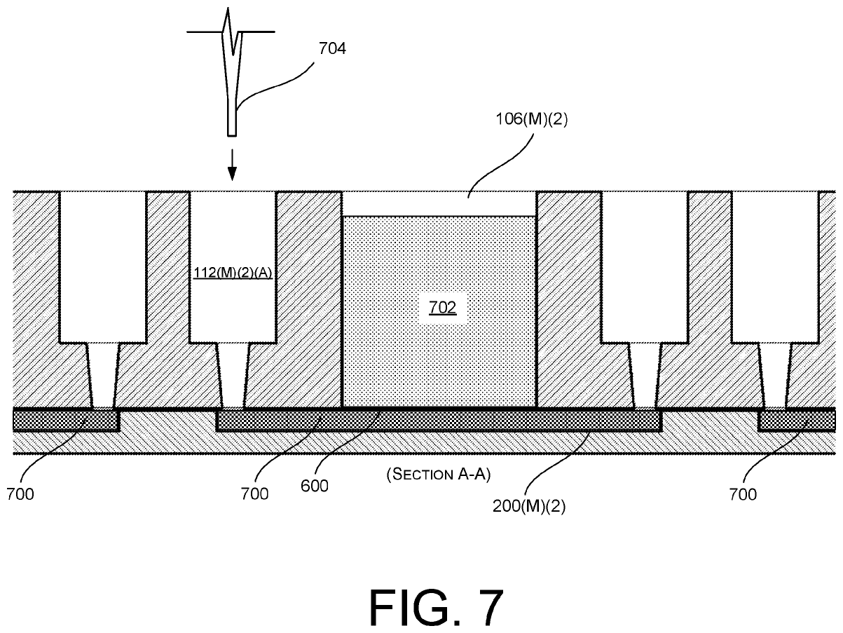

Turning to FIG. 7, there is illustrated the cross-sectional view of FIG. 6, but with hydrogel 700 in the hydrogel chambers 200, and with fluid media 702 (which may contain one or more optional compound(s), such as test compounds, assay components, etc.) in the media wells 106. FIG. 7 also illustrates a technique for loading the hydrogel chamber 200(M)(2) with a hydrogel 700 containing cells in order to seed the media wells 106 with cells embedded in hydrogel 700 by loading the hydrogel 700 into the hydrogel chambers 200 underneath the media wells 106. For example, a first (open) end of the loading port 112(M)(2)(A) may be configured to receive a pipette tip 704, or a similar tool. The pipette tip 704 may represent a tip of a pipette that is, prior to loading the hydrogel chamber 200(M)(2), filled with hydrogel 700 containing cells. For example, the hydrogel 700 (sometimes referred to herein as an “extracellular matrix (ECM) 700”) may be formed by cutting or breaking material (or elements) representing an endosteal niche of a bone marrow model into shards, and embedding or mixing the shards throughout a cell-laden hydrogel that represents a vascular niche of the bone marrow model. In some embodiments, the material representing the endosteal niche may include mineralized collagen. In some embodiments, the shards of mineralized collagen may encapsulate or contain cells found in the natural endosteal niche of normal bone, including, without limitation, osteoblasts, osteoprogenitors, and/or osteochondroprogenitors. In some embodiments, the cell-laden hydrogel in which the shards are embedded may itself encapsulate or contain cells found in the natural vascular (or stem cell, or hematopoietic) niche of normal bone, including, without limitation, hematopoietic stem cells (HSCs), long-term hematopoietic stem cells (LT-HSCs), short-term hematopoietic stem cells (ST-HSCs), multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), and/or other specialized marrow stromal populations such as CXCL12-abundant reticular (CAR) cells. In some embodiments, the microfluidic hydrogel chambers 200 may be configured to maintain relatively small volumes (e.g., 10 to 50 microliters (μL)). It is to be appreciated, however, that the height (or depth), H4, of the hydrogel chambers 200 can be designed as per the protocol requirements to create larger or smaller volumes. The hydrogel 700 that flows into the hydrogel chamber 200 takes the shape of the chamber to form a 3D matrix. This design also allows for loading of endothelial cells, for example, directly into the media wells 106, which may be a more reliable way to form a monolayer of those cells that is driven by gravity. In another embodiment, the “hydrogel chamber” houses cells in a media, essentially without a hydrogel component. This alternative configuration can be viewed as analogous to a miniaturized transwell plate systems, and can be used for assays and analyses otherwise applicable to such systems. Optionally, a hydrogel layer-membrane-media may be provided on top of the “hydrogel chamber”.

The media well 106(M)(2) may be filled with fluid media 702 using any suitable technique known to a person of ordinary skill in the art, such as by pouring fluid media 702 from a source container into the media well 106(M)(2). In some embodiments, an individual media well 106 is configured to hold fluid media 702 in a range of a few milliliters (mL) to 10 μL. Upon filling the media well 106(M)(2) with fluid media 702 and filling the hydrogel chamber 200(M)(2) with the hydrogel 700, the fluid media 702 remains generally segregated from the hydrogel 700 by virtue of the permeable barrier 600, except for the passage of nutrients, metabolites, waste, etc. through the permeable barrier 600. In some embodiments, the permeable barrier 600 may or may not include ECM lined with Endothelial Cells (on one or both sides). That is, open media wells 106 allow for loading appropriate cell culture media 702 as well as to perform batch-wise exchange to replenish individual wells 106. For assays that require assessing drug toxicities and efficacies, open top media wells 106 allows ease of automation for dosing any compounds at any desired time points using existing robotics (ease of integration with other plate-based technologies).

In some embodiments, the well plate 100 filled with contents (e.g., the fluid media 702 and the hydrogel 700 containing cells) may culture cells in one or more modes of operation. An example mode of operation is a “static mode” of operation, meaning that the well plate 100 is left to sit in an incubator or another controlled environment as-is, without any external forces applied to the well plate 100. In this static mode of operation, the fluid media 702 within the media wells 106 may remain generally motionless, or at least the fluid media 702 does not flow from the media well 106 to another chamber within the well plate 100. The hydrogel loading ports can optionally hold additional media, for instance to reduce or stop evaporation. Staining/labeling/detection molecules can be introduced via this port also, though it may take longer to diffuse through the system than when introduced through alternative paths. In other words, the well plate 100 filled with contents may be used for static assays by placing the well plate 100 in an incubator and leaving it for a period of time with the fluid media 702 and the hydrogel 700 remaining in the well plate 100. An operator may periodically take samples of the contents (e.g., the fluid media 702 and/or the hydrogel 700), may analyze the samples, and may determine analysis results (e.g., to determine how a drug is interacting with the cells). Each vertically-oriented set of features (e.g., a hydrogel chamber 200 filled with hydrogel 700, a permeable membrane 600, and a media well 106 filled with fluid media 702) may represent an isolated organ reactor (or “bioreactor”). The pair of loading ports 112 act as two inlet/outlet (I/O) ports for loading cells embedded in hydrogels 700.

Another example mode of operation is an “agitation (or stirring) mode” of operation. For example, an operator may mount the well plate 100 to an orbital shaker (or to any other suitable agitation mechanism, such as a rocker or similar device), and may operate the orbital shaker in order to agitate the fluid media 702 and/or the hydrogel 700 contained within the well plate 100. In this agitation mode of operation, at least the fluid media 702 in the media wells 106 may move, during agitation, throughout the volume of the media wells 106. Applied agitation may be continuous, periodic, intermittent (e.g., pulsed), etc. Another example of an agitation mechanism is an automated mixer, such as a stirring stick, that is mounted above a given media well 106, inserted into the media well 106 through the open top end of the media well 106, and operated to stir the fluid media 702 therein, which may be continuous, periodic, intermittent, etc. In this agitation mode of operation, an operator may periodically take samples of the contents (e.g., the fluid media 702 and/or the hydrogel 700), may analyze the samples, and may determine analysis results (e.g., to determine how a drug is interacting with the cells). Also contemplated are embodiments and uses wherein one or more detection/staining moieties are introduced into the fluid media, for instance for detecting (e.g., imaging) a cell or cell component or cell contents. Such detection/staining moieties include specific markers or proteins (including antibodies); other appropriate moieties are discussed herein and/or known in the art.

Returning with reference to FIG. 1, the media wells 106 of the well plate 100 may include respective top ends that open into a recessed area 114 at the top 102 of the well plate 100. For example, the well plate 100 may have a number, M, of recessed areas 114(1)-(M) defined in the top 102 of the well plate 100. The number M may correspond to the number of rows of media wells 106. Accordingly, a first row of media wells 106(1)(1)-(1)(N) may open into a first recessed area 114(1), a second row of media wells 106(2)(1)-(2)(N) may open into a second recessed area 114(2), and so on and so forth for any suitable number, M, of rows/recessed areas 114.

An individual recessed area 114 may be any suitable shape. In the example well plate 100 shown in FIG. 1, the recessed areas 114 are cuboidal, such as by forming a rectangular cutout of the top 102 of the well plate 100, the rectangle having a width longer than its height. In some embodiments, the recessed areas 114 may be rectangular and may substantially span an overall width, W1, of the well plate 100. A given recessed area 114 is separated from an adjacent recessed area 114 by a vertically-oriented wall 116. Accordingly, for a number, M, of recessed areas 114, there may be a number, M−1, of vertically-oriented walls 116 (not including the outer, perimeter walls of the well plate 100), where each wall 116 separates a pair of adjacent recessed areas 114.

An individual recessed area 114 may form an air chamber when a second well plate is coupled to the top 102 of the (first) well plate 100. This feature will be described in more detail below. Having recessed areas 114 separated by vertically-oriented walls 116 allows for pressurizing each air chamber separately and independently of one another, which may allow for implementing variability into the cell culturing conditions (e.g., different amounts of air pressure, different types of air pressure (e.g., negative (vacuum) or positive pressure), etc.).

The well plate 100 may further include one or more connectors 118 on, and extending from, an external surface of a side 120 of the well plate 100. FIG. 1 depicts a number, M, of connectors 118. This corresponds to the number, M, of rows of media wells 106, as well as the number, M, of recessed areas 114. In this manner, a given connector 118 may correspond to an individual recessed area 114 and to an individual row of media wells 106. Each connector 118 may be configured to connect to a pump for pressurizing an air chamber formed by the corresponding recessed area 114, as described in more detail below.

FIG. 4 further shows the well plate 100 as having an overall length, L1, and an overall width, W1. In some embodiments, the overall length, L1, of the well plate 100 is no greater than 100 millimeters (mm), no greater than 90 mm, no greater than 80 mm, no greater than 70 mm, or no greater than 60 mm. In some embodiments, the overall length, L1, of the well plate 100 is at least 60 mm, at least 70 mm, at least 80 mm, at least 90 mm, or at least 100 mm. In some embodiments, the overall length, L1, of the well plate 100 that allows for culturing cells at suitably-high throughput is in a range of 70 to 90 mm. In some embodiments, the overall width, W1, of the well plate 100 is no greater than 150 mm, no greater than 140 mm, no greater than 130 mm, no greater than 120 mm, or no greater than 110 mm. In some embodiments, the overall width, W1, of the well plate 100 is at least 110 mm, at least 120 mm, at least 130 mm, at least 140 mm, or at least 150 mm. In some embodiments, the overall width, W1, of the well plate 100 that allows for culturing cells at suitably-high throughput is in a range of 120 to 140 mm. In some embodiments, a 5×5 array of media wells 106 is implemented on a well plate 100 having an overall width, W1, of 130 mm, and an overall length, L1, of 80 mm.

FIG. 4 also shows a vertically-oriented wall 116 as having a thickness, T1. In some embodiments, the thickness, T1, of an individual vertically-oriented wall 116 is no greater than 3 mm, no greater than 2 mm, or no greater than 1 mm. In some embodiments, the thickness, T1, of an individual vertically-oriented wall 116 is at least 1 mm, at least 2 mm, or at least 3 mm. The outer (perimeter) walls of the well plate 100 may be of a similar thickness to the thickness, T1, of a vertically-oriented wall 116 that separates a pair of adjacent recessed areas 114 at the top 102 of the well plate 100.

FIG. 2 also shows a recessed area 114 as having a height (or depth), H1. In some embodiments, the height, H1, of an individual recessed area 114 is no greater than 7 mm, no greater than 6 mm, no greater than 5 mm, no greater than 4 mm, or no greater than 3 mm. In some embodiments, the height, H1, of an individual recessed area 114 is at least 3 mm, at least 4 mm, at least 5 mm, at least 6 mm, or at least 7 mm.

Referring again to FIG. 4, an individual connector 118 is shown as having a length, L2. In some embodiments, the length, L2, of an individual connector 118 is no greater than 12 mm, no greater than 11 mm, no greater than 10 mm, no greater than 9 mm, or no greater than 8 mm. In some embodiments, the length, L2, of an individual connector 118 is at least 8 mm, at least 9 mm, at least 10 mm, at least 11 mm, or at least 12 mm. An individual connector 118 may also have a first outer diameter, D4, at a distal end of the connector 118. In some embodiments, the first outer diameter, D4, at a distal end of the connector 118 is about 2 mm. An individual connector 118 may also have a second outer diameter, D5, at a proximal end of the connector 118. In some embodiments, the second outer diameter, D5, at a proximal end of the connector 118 is about 4 mm. Accordingly, the outer diameter of the connector 118 may progressively narrow from the proximal end of the connector 118 to the distal end of the connector 118. In some embodiments, a connector 118 may be a Luer connector. An individual connector 118 may also have an inner diameter, D6, running through the length of the connector 118 and in fluid communication with an aperture in the side 120 of the well plate 100. In some embodiments, the inner diameter, D6, of the connector 118 is about 1 mm. The aperture in the side 120 of the well plate 100 may have a diameter, D7, that is about 1.5 mm. This allows for air to ingress or egress to/from the connector 118 for pressurizing the air chamber that is formed by the recessed area 114 at the top of the well plate 100.

FIG. 3 shows the first substrate 108 of the well plate 100 as having a height, H2. In some embodiments, the height, H2, of the first substrate 108 is no greater than 17 mm, no greater than 16 mm, no greater than 15 mm, no greater than 14 mm, or no greater than 13 mm. In some embodiments, the height, H2, of the first substrate 108 is at least 13 mm, at least 14 mm, at least 15 mm, at least 16 mm, or at least 17 mm. The second substrate 110 of the well plate 100 may have a height of, H3. In some embodiments, the height, H3, of the second substrate 110 is no greater than 3 mm, no greater than 2 mm, or no greater than 1 mm. In some embodiments, the height, H3, of the second substrate 110 is at least 1 mm, at least 2 mm, or at least 3 mm.

FIG. 2 shows an individual media well 106 defined in the well plate 100 as having an inner diameter, D8. In some embodiments, the inner diameter, D8, of an individual media well 106 is no greater than 11 mm, no greater than 10 mm, no greater than 9 mm, no greater than 8 mm, or no greater than 7 mm. In some embodiments, the inner diameter, D8, of an individual media well 106 is at least 7 mm, at least 8 mm, at least 9 mm, at least 10 mm, or at least 11 mm. In some embodiments, the inner diameter, D8, of an individual media well 106 is a constant diameter. FIG. 2 also shows an individual hydrogel chamber 200 as having a height (or depth), H4. In some embodiments, the height, H4, of an individual hydrogel chamber is about 1 mm.

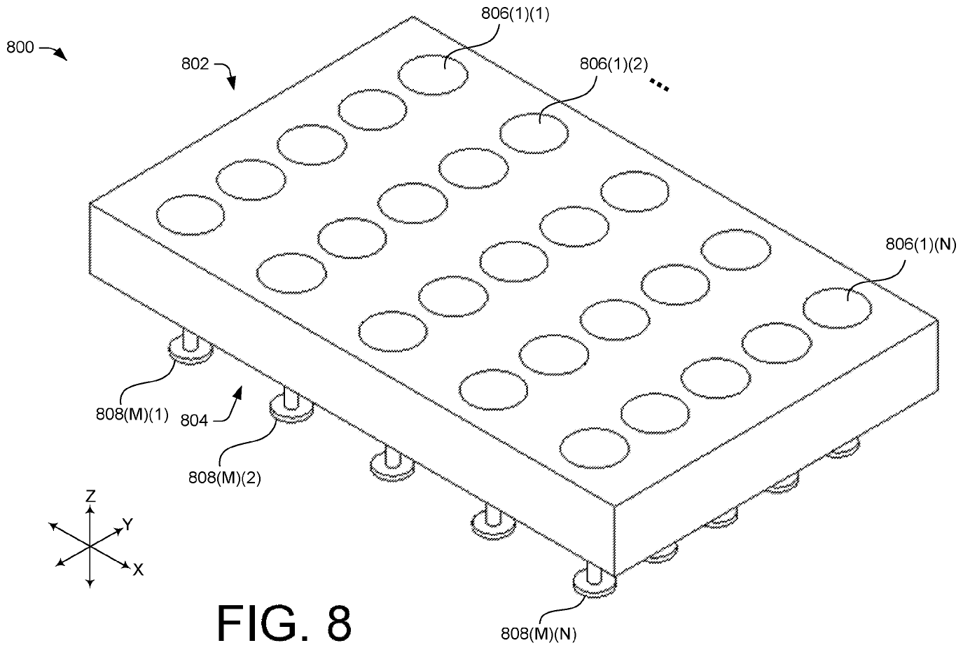

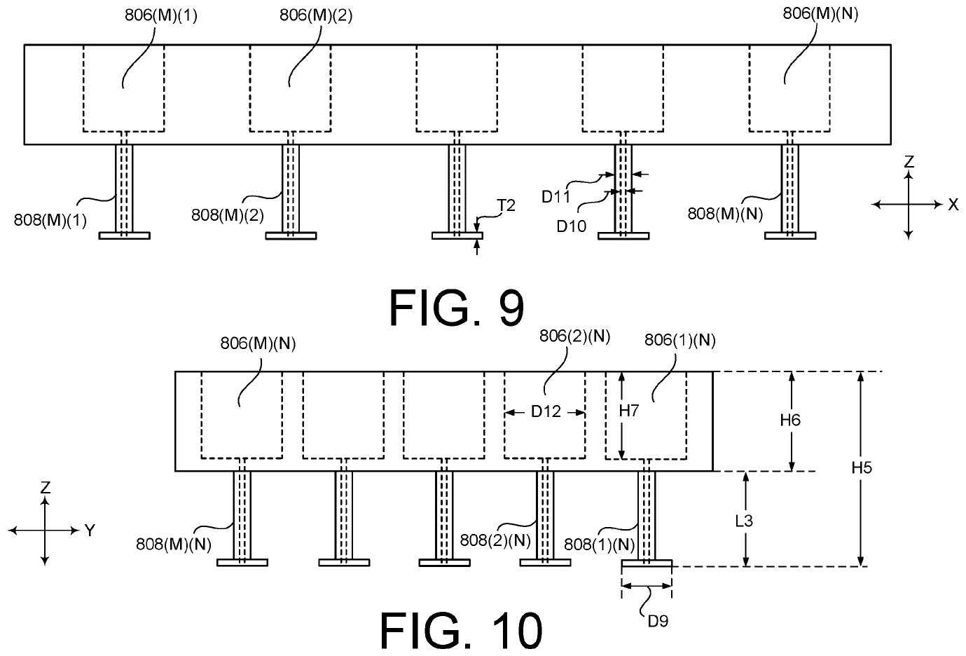

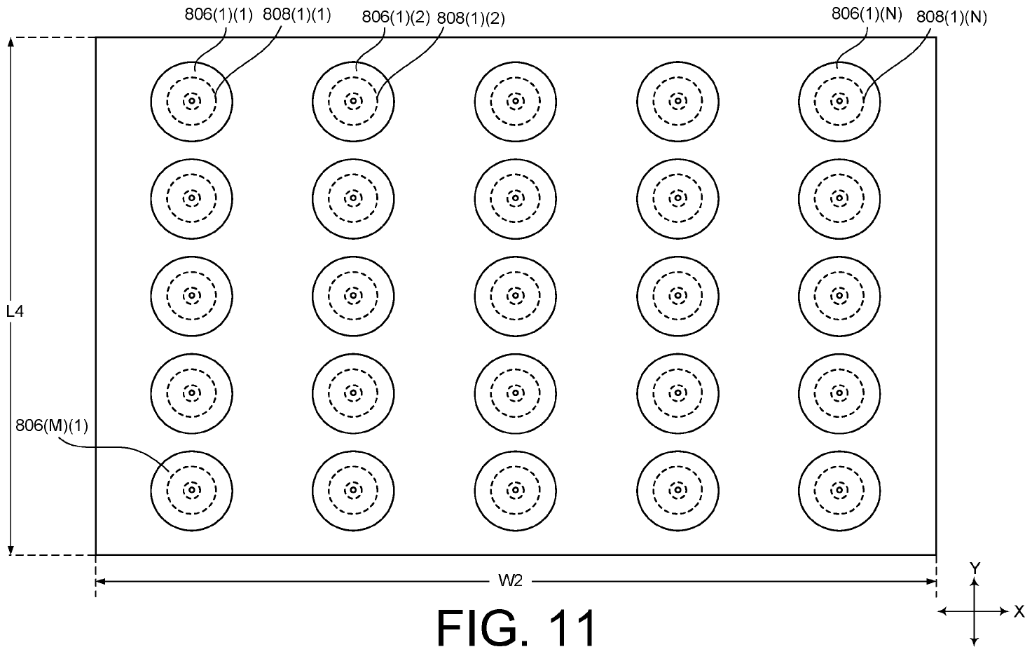

Referring to FIG. 8, there is illustrated an example second well plate 800 that is configured to detachably couple to a top of the example (first) well plate 100 of FIG. 1. The second well plate 800 is shown from a perspective view in FIG. 8. FIG. 9 illustrates a front view of the example second well plate 800 of FIG. 8, FIG. 10 illustrates a side view of the example second well plate 800 of FIG. 8, and FIG. 11 illustrates a top view of the example second well plate 800 of FIG. 8. The second well plate 800 may be part of a larger system that also includes the first well plate 100, as will be described in more detail below.

The second well plate 800 has a top 802 and a bottom 804, as well as four sides. Accordingly, the second well plate 800 is generally cuboidal. It is to be appreciated, however, that the second well plate 800 disclosed herein is not limited to being cuboidal, as the second well plate 800 may be of any polygonal shape, such as cylindrical- or disk-shaped with a single continuous side surface between the top 802 and bottom 804, triangular-shaped with three sides between the top 802 and bottom 804, pentagonal-shaped with five sides between the top 802 and the bottom 804, and so on.

As shown in FIG. 8, the second well plate 800 may include an M×N array of collection wells 806, with M rows of collection wells 806 and N columns of collection wells 806, where M can be any suitable integer and N can be any suitable integer. The example M×N array of collection wells 806 shown in FIG. 8 is a 5×5 array of collection wells 806 where M=5 and N=5. With a first well plate 100 having a 5×5 array of media wells 106, this provides a one-to-one correspondence of media wells 106 to collection wells 806 due to the grid-like arrangement of both arrays of wells in the respective well plates.

The M×N array of collection wells 806 is in fluid communication with an M×N array of tubes 808 extending from a bottom surface of the second well plate 800. That is, an individual collection well 806 is in fluid communication with a corresponding tube 808. An individual collection well 806 is configured to receive fluid media via its corresponding tube 808 in a “continuous perfusion mode” of operation, which is described in more detail below.

The second well plate 800 may be made of any suitable material, combination of materials, or composite materials. For example, the second well plate 800 can be made of any of the polymer/plastic materials described herein, such as a laboratory-grade plastic including, without limitation, polycarbonate plastic, polystyrene, polyethylene, PET, COC, COPs and the like. It is to be appreciated that the second well plate 800 can be made of any other suitable rigid or semi-rigid material that is suitable for cell culturing.

In some embodiments, the second well plate 800 may be manufactured using an injection molding technique, or an extrusion technique, the processes for which should are known to a person having ordinary skill in the art. Other manufacturing techniques that may be used to manufacture the second well plate 800 include machining a material into the shape of the second well plate 800, or into component parts of the second well plate 800 that are attached together during manufacture using any suitable fastening means, such as screws, pins, joints, adhesives, or the like. Any other subtractive manufacturing techniques can be used besides machining. Additionally, additive manufacturing techniques, such as 3D printing, can be used to manufacture the second well plate 800.

An individual tube 808 extending from a bottom of the second well plate 800 is shown in FIG. 10 as having an annular flange with an outer diameter, D9. This outer diameter, D9, may be less than an inner diameter, D8, of a corresponding media well 106 in the first well plate 100. This allows the tube 808 to be inserted into the media well 106 when the second well plate 800 is coupled to the top 102 of the first well plate 100. In some embodiments, the outer diameter, D9, of an individual tube 808 (at the annular flange of the tube 808) is no greater than 9.5 mm, no greater than 8.5 mm, no greater than 7.5 mm, no greater than 6.5 mm, or no greater than 5.5 mm. In some embodiments, the outer diameter, D9, of an individual tube 808 (at the annular flange of the tube 808) is at least 5.5 mm, at least 6.5 mm, at least 7.5 mm, at least 8.5 mm, or at least 9.5 mm.

FIG. 10 also shows an individual tube 808 as having length of, L3. In some embodiments, the length, L3, of an individual tube 808 is no greater than 16.25 mm, no greater than 15.25 mm, no greater than 14.25 mm, no greater than 13.25 mm, or no greater than 12.25 mm. In some embodiments, the length, L3, of an individual tube 808 is at least 12.25 mm, at least 13.25 mm, at least 14.25 mm, at least 15.25 mm, or at least 16.25 mm.

FIG. 9 shows an individual tube 808 as having an inner diameter, D10. In some embodiments, the inner diameter, D10, of an individual tube 808 is about 0.75 mm. FIG. 9 also shows an individual tube 808 as having second outer diameter, D11, at a section of the tube 808 above the annular flange, which may be about 2.5 mm. FIG. 9 also shows a thickness, T2, of the annular flange of the tube 808, which may be about 1 mm.

FIG. 10 shows an individual collection well 806 as having an inner diameter, D12. In some embodiments, the inner diameter, D12, of an individual collection well 806 is no greater than 14.45 mm, no greater than 13.45 mm, no greater than 12.45 mm, no greater than 11.45 mm, or no greater than 10.45 mm. In some embodiments, the inner diameter, D12, of an individual collection well 806 is at least 10.45 mm, at least 11.45 mm, at least 12.45 mm, at least 13.45 mm, or at least 14.45 mm.

FIG. 10 also shows the second well plate 800 as having an overall height, H5. In some embodiments, the overall height, H5, of the second well plate 800 is no greater than 33.25 mm, no greater than 31.25 mm, no greater than 29.25 mm, no greater than 27.25 mm, or no greater than 25.25 mm. In some embodiments, the overall height, H5, of the second well plate 800 is at least 25.25 mm, at least 27.25 mm, at least 29.25 mm, at least 31.25 mm, or at least 33.25 mm. The height, H6, of an upper section of the second well plate 800 may be no greater than 19 mm, no greater than 17 mm, no greater than 15 mm, no greater than 13 mm, or no greater than 11 mm. In some embodiments, the height, H6, of the upper section of the second well plate 800 is at least 11 mm, at least 13 mm, at least 15 mm, at least 17 mm, or at least 19 mm. The height (or depth), H7, of an individual collection well 806 may be no greater than 17 mm, no greater than 15 mm, no greater than 13 mm, no greater than 11 mm, or no greater than 9 mm. In some embodiments, the height (or depth), H7, of an individual collection well 806 is at least 9 mm, at least 11 mm, at least 13 mm, at least 15 mm, or at least 17 mm.

FIG. 11 further shows the second well plate 800 as having an overall length, L4, and an overall width, W2. In some embodiments, the overall length, L4, of the second well plate 800 is no greater than 100 millimeters (mm), no greater than 90 mm, no greater than 80 mm, no greater than 70 mm, or no greater than 60 mm. In some embodiments, the overall length, L4, of the second well plate 800 is at least 60 mm, at least 70 mm, at least 80 mm, at least 90 mm, or at least 100 mm. In some embodiments, the overall length, L4, of the second well plate 800 that allows for culturing cells at suitably-high throughput is in a range of 70 to 90 mm. In some embodiments, the overall width, W2, of the second well plate 800 is no greater than 150 mm, no greater than 140 mm, no greater than 130 mm, no greater than 120 mm, or no greater than 110 mm. In some embodiments, the overall width, W2, of the second well plate 800 is at least 110 mm, at least 120 mm, at least 130 mm, at least 140 mm, or at least 150 mm. In some embodiments, the overall width, W2, of the second well plate 800 that allows for culturing cells at suitably-high throughput is in a range of 120 to 140 mm. In some embodiments, a 5×5 array of collection wells 806 is implemented on a second well plate 800 having an overall width, W2, of 130 mm, and an overall length, L4, of 80 mm.

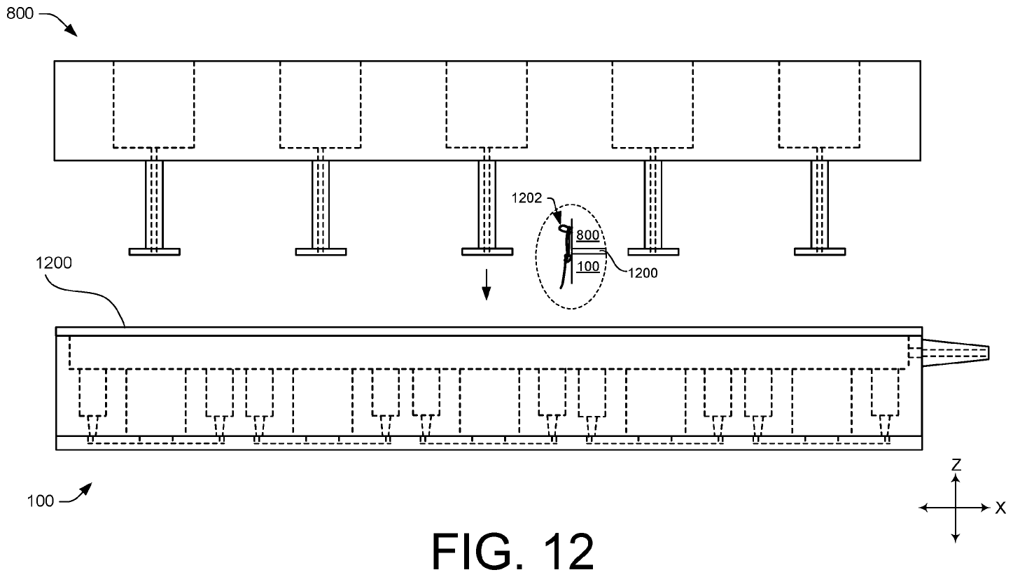

FIG. 12 illustrates a method of detachably coupling the example second well plate 800 of FIG. 8 to a top 102 of the example (first) well plate 100 of FIG. 1. As depicted in FIG. 12, the second well plate 800 may be brought down upon the top 102 of the first well plate 100 from above. This may be done after filling the first well plate 800 with its contents (e.g., fluid media 702 and hydrogel 700 containing cells), as described herein. FIG. 12 also shows an elastomer 1200 to create a hermetic seal that is reversable when the second well plate 800 is coupled to the top 102 of the first well plate 100. Any suitable elastomer 1200 may be utilized, such as a rubber O-ring, a gasket, or the like that runs the perimeter of the first well plate 100 and/or the second well plate 800; a magnetic coupling is also possible. FIG. 12 shows an example of an elastomer 1200 that runs the perimeter of the first well plate 100 where the two well plates 100/800 matingly engage. The weight of the second well plate 800 may in some instances be sufficient for creating the hermetic seal. In some embodiments, a lock 1202 (or a latch 1202) may be provided on an external side surface to press the second well plate 800 into a firm engagement with the first well plate 100. For example, a two-part mechanism, such as a hook(s) on one well plate may latch onto a pin on the other well plate. The latch 1202 in FIG. 12 is depicted as a type of latch 1202 that can be found on a jar lid, but any suitable latching mechanism can be utilized. Such a lock 1202 (or latch 1202) may be provided on one or more sides of the well plate(s) 100/800.

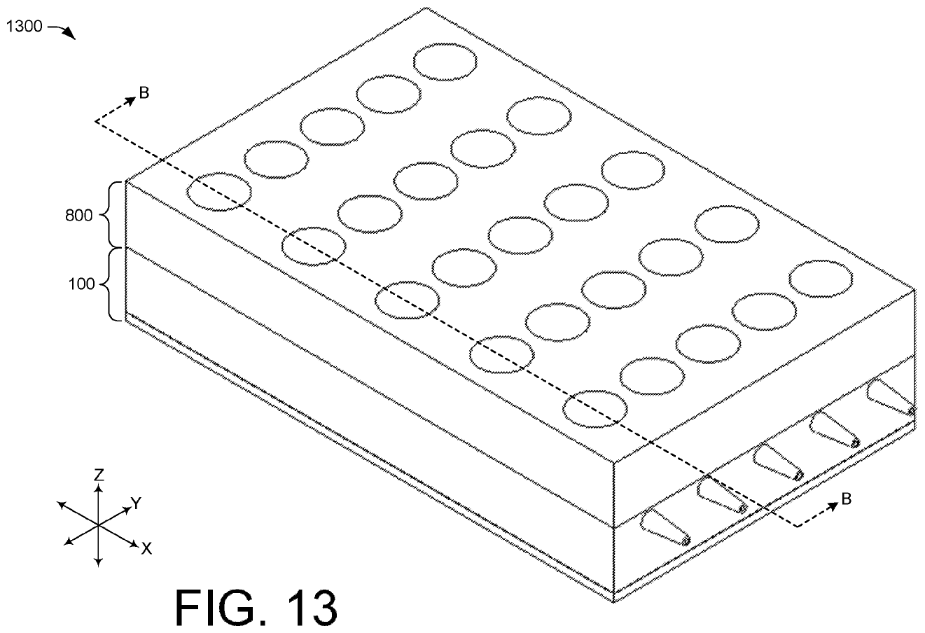

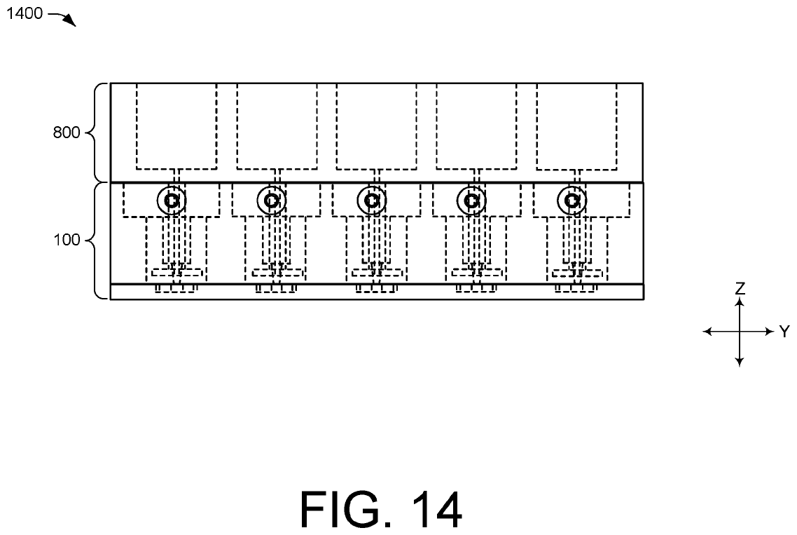

FIG. 13 illustrates a system 1300 including the example second well plate 800 of FIG. 8 coupled to the example (first) well plate 100 of FIG. 1. This system 1300 allows for culturing cells in a continuous perfusion mode of operation, as described in more detail below. FIG. 14 illustrates a side view of the example system 1300 of FIG. 13, FIG. 14 showing internal features of the system in dashed lines. These features were described earlier and will not be described again with reference to FIG. 14 for the sake of brevity.

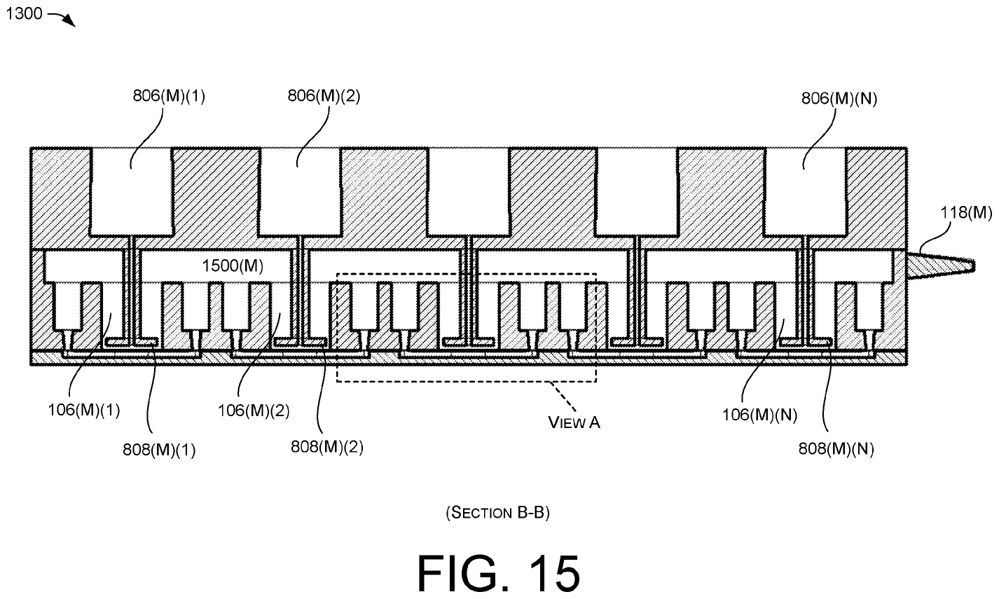

FIG. 15 illustrates a cross-sectional view of the example system 1300 of FIG. 13, taken along section line B-B. As depicted in FIG. 15, when the second well plate 800 is coupled to the top 102 of the first well plate 100, an individual collection well 806 of the second well plate 800 is positioned over (or atop) a corresponding media well 106 of the first well plate 100. For example, the collection well 806(M)(1) is positioned over the media well 106(M)(1) in FIG. 15. Furthermore, the recessed area 114(M) at the top 102 of the first well plate 100 forms an air chamber 1500(M) when the second well plate 800 is coupled to the top 102 of the first well plate 100, as shown in FIG. 15. Accordingly, multiple air chambers 1500(1)-(M) may be formed by the multiple recessed areas 114(1)-(M) when the second well plate 800 is coupled to the top 102 of the first well plate 100. The individual air chambers 1500 are configured to be pressurized independently from other air chambers 1500 through operation of a pump (or another similar system, such as a compressed air tank with a standard regulator) that is connected to a corresponding connector 118. In the case of FIG. 15, the connector 118(M) corresponds to the air chamber 1500(M). Though illustrated with the “air chamber” forming between the top and bottom plate, also contemplated are embodiments wherein air chamber is formed on the top of top plate; optionally, a system can have both types of air chamber. One benefit of such embodiments is that they enable both plates to have their own air pressure chamber.

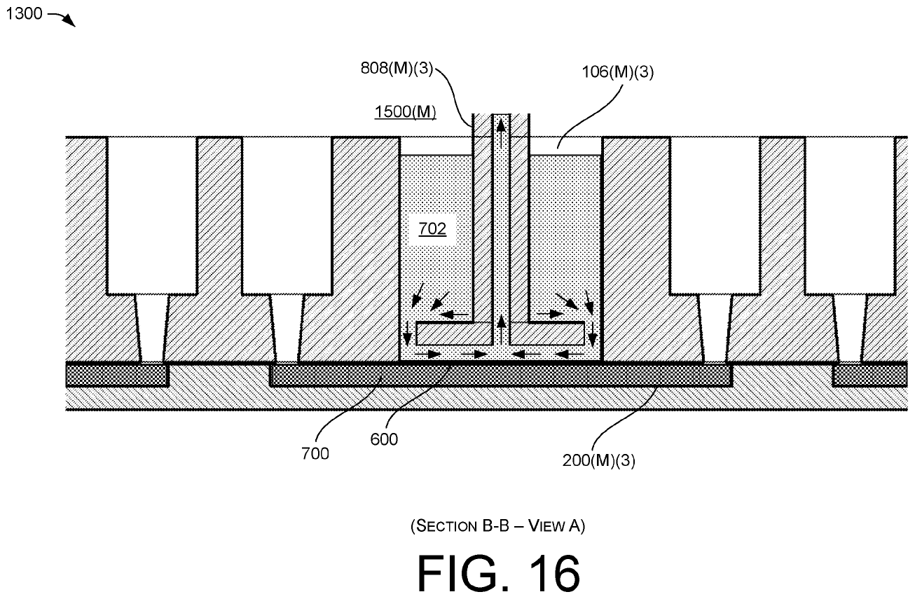

FIG. 16 illustrates a zoomed in view (View A) of a portion of the cross-sectional view of FIG. 15. FIG. 16 shows the system 1300 operating in a continuous perfusion mode of operation, which may be in accordance with certain cell culture protocols. As shown in FIG. 16, a microfluidic channel is formed in the tube 808(M)(3) by virtue of the second well plate 800 being coupled to the first well plate 100, and also by virtue of the air chamber 1500(M) being pressurized. That is, the pressurized air chamber 1500(M) causes continuous perfusion of the fluid media 702 through the tubes 808 associated with that air chamber 1500(M), including the tube 808(M)(3). The depth of the microfluidic channel formed can be influenced by the changing the length of sipping tube 808; a longer tube will result in a narrower microfluidic channel. Varying the depth of the microfluidic channel allows modulation of shear stress, for instance on cells (such as endothelial cells) that are deposited on the membrane. The pressurized air chamber 1500(M) may be pressurized with a positive pressure. A positive pressure in the air chamber 1500(M) may be created by operating a pump that pushes or pumps air into the air chamber 1500(M). Alternatively, a pump may be operated in a reverse direction to create a vacuum in the hermetically-sealed air chamber 1500(M) The amount of pressure (positive or negative) may be controlled (e.g., by a pressure controller that is part, or connected to, the pump) to cause a desired rate of perfusion. The system also enables control of the directionality of media flow, and the media can be recirculated the between top and bottom of the device or the chamber. Because the tube 808(M)(3) is in fluid communication with a collection well 806(M)(3) disposed above the tube 808(M)(3), fluid media 702 that originates in the media well 106(M)(3) may travel up through the tube 808(M)(3) (via a through-hole in the center of the tube 808(M)(3)) and may empty (or outflow) into the collection well 806(M)(3) during the continuous perfusion mode of operation. In some embodiments, directionality of the flow is configurable. Positive pressure in pressurized air chamber 1500 will cause media to flow bottom to top and negative pressure will reverse it; this enables recirculation of the media. This is beneficial, for instance, since the media will not be saturated with material coming from cells. However, regardless of whether positive or negative pressure is used to pressurize the air chamber(s) 1500, the pressurized air chamber 1500 may cause the fluid media 702 to flow in an upward direction through the tube 808(M)(3) and into a corresponding collection well 806(M)(3). This flow direction is depicted in FIG. 16. For example, the flow of media 702 may be in a radially-inwards direction along the porous membrane 600. The pressure gradient to create this flow is created by the pressurized air chamber 1500, which is pressurized using any suitable external compressed air source (e.g., a pump). When the air chamber 1500 is pressurized, the media 702 in each media well 106 will start flowing towards the outflow collection wells 806 through the tubes 808. The design of the tube 808 geometry can be changed to modulate the fluid stresses experienced by the cells on/near membrane 600. It is to be appreciated that these conditions may occur for the multiple media wells 106 in a given row that open into a common air chamber 1500. Accordingly, the system 1300 is operable in a continuous perfusion mode where a one-to-one association allows the media 702 originating in each media well 106 to be collected in a separate collection well 806 through the tube 808, thereby avoiding cross contamination between bioreactors. The system 1300 can also control the local shear stresses and mass transport rates by controlling the flow rates using a variable pressure source. Again, each vertically-oriented set of features (e.g., a hydrogel chamber 200 filled with hydrogel 700, a permeable membrane 600, a media well 106 filled with fluid media 702, a tube 808, and a collection well 806) may represent an isolated organ reactor (or “bioreactor”). In the examples shown in the figures, the system 1300 includes 25 organ bioreactors in a 5×5 array. Each row of the bioreactors shares a single pressure source when operated in the continuous perfusion mode of operation.

The processes described herein are illustrated as a collection of blocks in a logical flow graph, which represent a sequence of operations. The order in which the operations are described is not intended to be construed as a limitation, and any number of the described blocks can be combined in any order and/or in parallel to implement the processes.

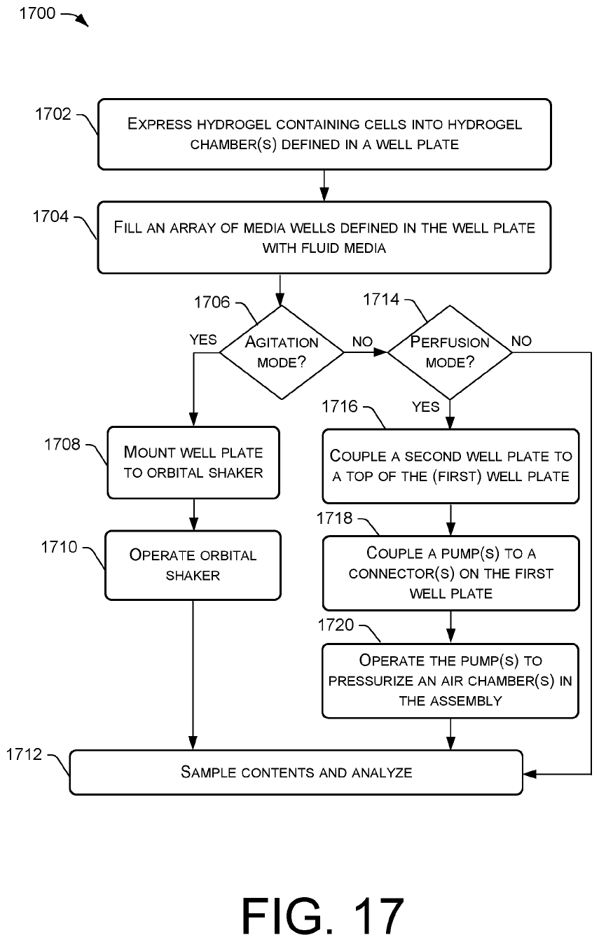

FIG. 17 illustrates a flow diagram of an example process 1700 of using a well plate system for cell culturing. For discussion purposes, the process 1700 is described with reference to the previous figures.

At 1702, an operator may express, into an array of loading ports 112 in a well plate 100, a hydrogel 700 containing cells to at least partially fill an array of hydrogel chambers 200 in the well plate 100 with the hydrogel 700. FIG. 7 at least partially depicts this operation at block 1702 where a pipette tip 704 of a pipette containing hydrogel is inserted into a loading port 112 and operated to express hydrogel 700 containing cells into a hydrogel chamber 200.

At 1704, an operator may fill, at least partially, an array of media wells 106 in the well plate with fluid media 702. A media well 106 filled with fluid media 702 is also depicted in FIG. 7, by way of example.

At 1706, a determination may be made as to whether the well plate 100 (or system 100) is to be operated in agitation mode. If the agitation mode of operation is to be carried out, the process 1700 may follow the “YES” route from block 1706 to block 1708.

At 1708, an operator may mount the well plate 100 (filled with contents) to an orbital shaker, or to a similar agitation mechanism, and at 1710, the operator may operate the orbital shaker (or similar agitation mechanism) to agitate the fluid media 702 and/or the hydrogel 700 in the well plate (or system 100). As discussed herein, agitation may be performed continuously, periodically, intermittently, etc. At some point, at 1712, an operator may sample contents (e.g., the hydrogel 700 containing cells and/or the fluid media 702) to analyze the sampled contents and determine analysis results (e.g., to determine how a drug is interacting with cells). The well plate 100 may be placed in an incubator as part of any of the operational modes described herein.

Returning to block 1706, if the agitation mode of operation is not to be carried out, the process 1700 may follow the “NO” route from block 1706 to block 1714.

At 1714, a determination may be made as to whether the well plate 100 (or system 100) is to be operated in continuous perfusion mode. If the continuous perfusion mode of operation is to be carried out, the process 1700 may follow the “YES” route from block 1714 to block 1716.

At 1716, an operator may couple a second well plate 800 to the top 102 of the first well plate 100 to convert a recessed area(s) 114 defined in the top 102 of the first well plate 100 into a hermetically-sealed air chamber(s) 1500. This coupling at block 1716 may involve aligning the tubes 808 with the media wells 106, as well as locking, latching, and/or clamping the two well plates together using a lock 1202 (or a latch 1202) to help press the second well plate 800 firmly down upon the first well plate 100. In other embodiments, gravity may be sufficient to create a hermetically-sealed air chamber(s) 1500 in the assembled system 1300.

At 1718, an operator may couple a pump to a connector(s) 118 on an external side 120 surface of the first well plate 100, and at 1720, the operator may operate the pump to pressurize the hermetically-sealed air chamber(s) 1500 as a pressurized air chamber(s) 1500. The pressurized air may be pressurized at a negative pressure (to create a vacuum) or a positive pressure.

At 1712, following block 1720, an operator may sample contents (e.g., the hydrogel 700 containing cells and/or the fluid media 702) to analyze the sampled contents and determine analysis results (e.g., to determine how a drug is interacting with cells). This may be done at any suitable time, as described herein. The system 1300 may be placed in an incubator as part of the perfusion mode of operation.

Returning to block 1714, if the continuous perfusion mode of operation is not to be carried out, the process 1700 may follow the “NO” route from block 1714 directly to block 1712 to sample contents and to analyze the sampled contents in a static mode of operation. Again, this may be done at any suitable time, as described herein, and it may involve placing the well plate 100 filled with contents within an incubator.

The well plate 100 described herein represents a microfluidic, modular well-plate 100 for culturing of three-dimensional (3D) cell cultures embedded in supporting hydrogels 700 (or ECMs 700) in a static and/or agitation/stirring mode of operation. Additionally, the system 1300 described herein represents a microfluidic, modular well-plate assembly that constitutes a laminar perfusion/flow system for culturing 3D cell cultures embedded in supporting hydrogels 700 (or ECMs 700) in flow (continuous perfusion) mode of operation. Accordingly, this modular well plate design enables at least three modes of operation to create three different media conditions—(i) static, (ii) agitation/stirring, and (iii) unidirectional media exchange, providing enhanced flexibility and versatility compared to existing systems. This modular plate is usable for any 2D/3D cell in ECM 700, hydrogels 700, growth factors, any other microenvironment architecture using small volumes, and it enables testing of several experimental conditions simultaneously. Another optional mode employs cells without hydrogels. In the continuous perfusion mode of operation, a flow strategy is enabled where failure in one well 106 and/or tube 808 (clogging, bubbles) does not impact flow rates in other wells. The well plate 100 and/or system 1300 also provides capacity for scaling up to larger arrays of media wells 106 with a lower systemic failure rates. The system 1300 also allows for actively controlling the shear rate experienced by cells by changing the dimensions of the tubes 808.

Notably, existing commercial organ-on-chip systems are capable of a maximum throughput of about 24 or 48 experiments in parallel; in that format, 24 experiments run in parallel would be quite bulky and complex using off-chip components, likely requiring an entire incubator of space. The well plate 100 and/or system 1300 described herein is configured to scale up the number of experiments beyond this current limitation while keeping the cost per assay relatively low. Specifically, the disclosed microfluidic plate-based approach for culturing organ models addresses the issues of scalability and miniaturization simultaneously, thereby offering a solution that the pharmaceutical industry is more likely to adopt into their drug discovery pipeline. Furthermore, the disclosed technology is designed in a manner so that it is seamlessly integral with the existing liquid handling and robotics used in the pharmaceutical industry.

The modular, microfluidic well plate 100 and/or well plate system 1300 is configured to culture 3D, hydrogel-based systems; they are also useful to perform non-hydrogel based transwell type assays, but in low volumes. The plate-based approach enables further miniaturization, is inherent scalability, and can be seamlessly integrated with already-developed liquid handling robotics—a distinct advantage that existing organ-on-chip technologies cannot offer. Although bone marrow models are discussed herein as a suitable application for the well plate 100 and/or the system 1300, the well plate 100 and/or the system 1300 described herein may be utilized for other types of organoid models besides bone marrow models.

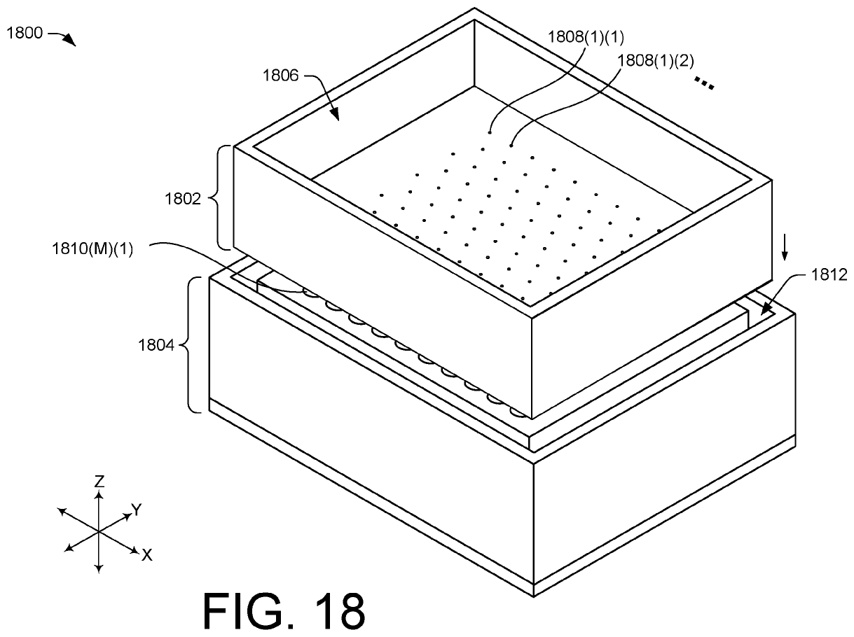

Referring to FIG. 18, there is illustrated an example system 1800 (sometimes referred to herein as a “gravity-based sample box 1800”), according to an example embodiment. The system 1800 is shown from a perspective view in FIG. 18, and it is comprised of a first reservoir 1802 (or “upper reservoir 1802”) and a second reservoir 1804 (or “outlet reservoir 1804”).

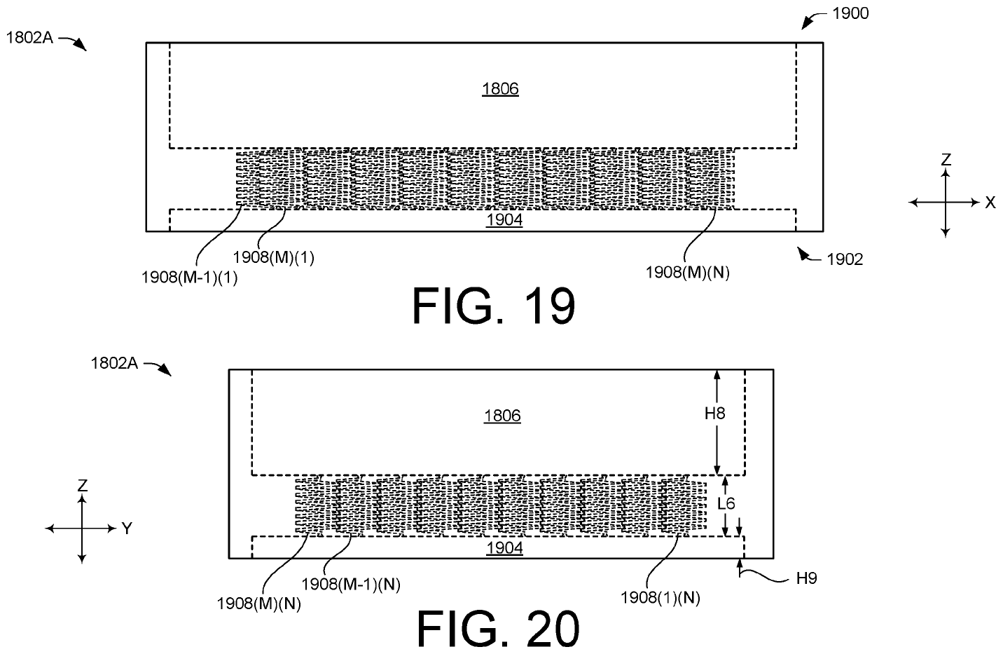

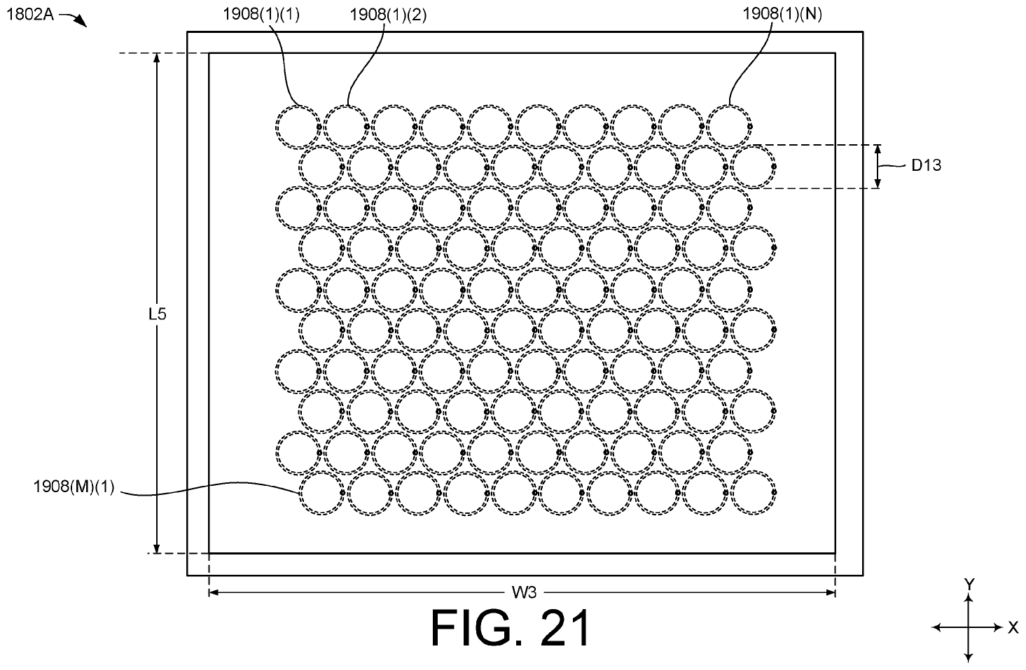

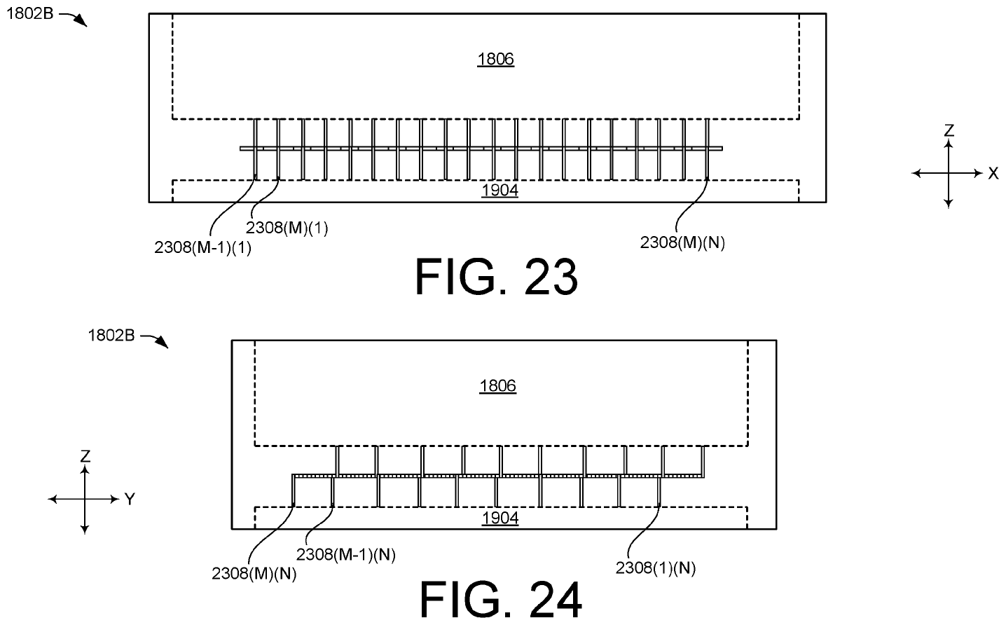

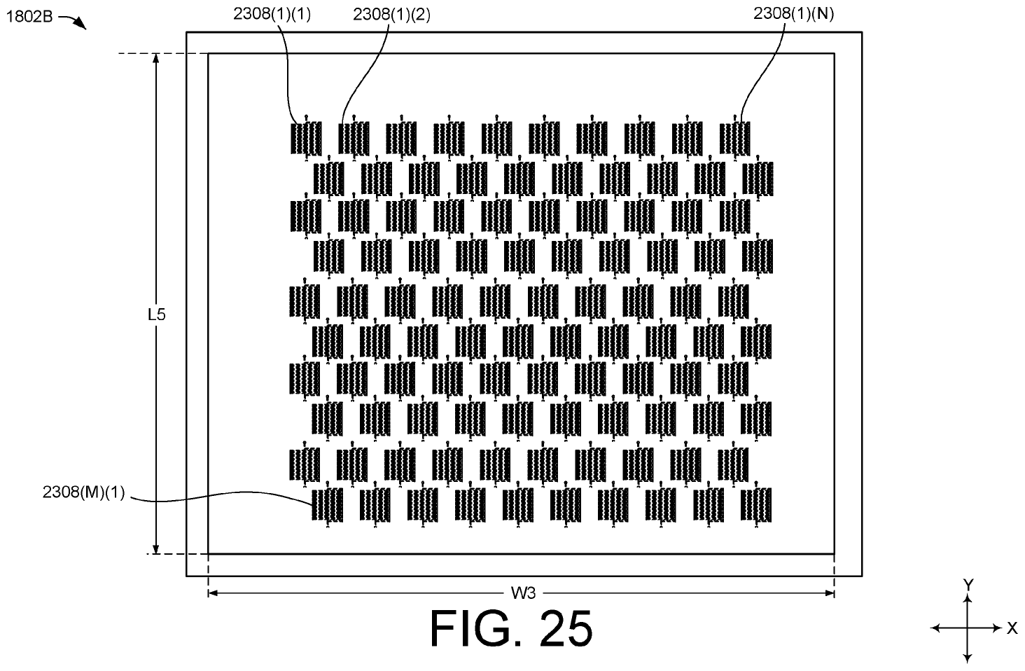

FIG. 19 illustrates a front view of the example first reservoir 1802A of FIG. 18, according to one embodiment where an array of microfluidic channels is in the form of spiral channels. FIG. 20 illustrates a side view of the example first reservoir 1802A of FIG. 18, according to the spiral channel embodiment, and FIG. 21 illustrates a top view of the example first reservoir 1802A of FIG. 18, according to the spiral channel embodiment. Meanwhile, FIG. 23 illustrates a front view of the example first reservoir 1802B of FIG. 18, according to another embodiment where an array of microfluidic channels is in the form of serpentine channels. FIG. 24 illustrates a side view of the example first reservoir 1802B of FIG. 18, according to the serpentine channel embodiment, and FIG. 25 illustrates a top view of the example first reservoir 1802B of FIG. 18, according to the serpentine channel embodiment.

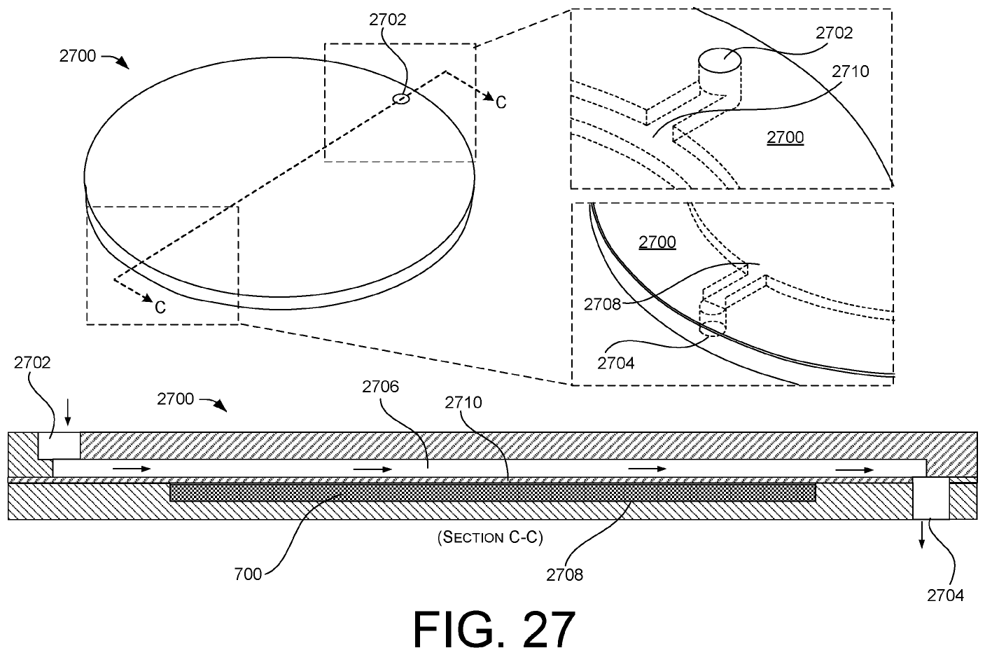

The first reservoir 1802 is configured to have installed therein/thereon an array of inserts that contain cells embedded in hydrogel (or an ECM) separated from a media channel by a permeable barrier. An example of such an insert is depicted in FIG. 27, and the insert will be described in more detail with reference to that figure. As indicated by the downward-directed arrow in FIG. 18, the first reservoir 1802 is configured to be placed atop the second reservoir 1802 so that fluid media received in the first reservoir 1802 can be collected in the second reservoir 1804 when the fluid media egresses from the bottom of the first reservoir 1802. When the array of inserts are installed in the first reservoir 1802, the gravity-based sample box 1800 is configured to culture cells, such as bone marrow cells.

The first reservoir 1802 has a top 1900 and a bottom 1902 (See FIG. 19), as well as four sides. Accordingly, the first reservoir 1802 is generally cuboidal. It is to be appreciated, however, that the first reservoir 1802 disclosed herein is not limited to being cuboidal, as the first reservoir 1802 may be of any polygonal shape, such as cylindrical- or disk-shaped with a single continuous side surface between the top 1900 and bottom 1902, triangular-shaped with three sides between the top 1900 and bottom 1902, pentagonal-shaped with five sides between the top 1900 and the bottom 1902, and so on.

The first reservoir 1802 may include a recessed area 1806 defined in the top 1900 of the first reservoir 1802. FIGS. 21 and 25 each show the recessed area 1806 as having a length, L5, and a width, W3. In some embodiments, the length, L5, of the recessed area 1806 in the top 1900 of the first reservoir 1802 is no greater than 135 mm, no greater than 125 mm, no greater than 115 mm, no greater than 105 mm, or no greater than 95 mm. In some embodiments, the length, L5, of the recessed area 1806 in the top 1900 of the first reservoir 1802 is at least 95 mm, at least 105 mm, at least 115 mm, at least 125 mm, or at least 135 mm. In some embodiments, the length, L5, of the recessed area 1806 in the top 1900 of the first reservoir 1802 that allows for culturing cells at suitably-high throughput is in a range of 110 to 120 mm. In some embodiments, the width, W3, of the recessed area 1806 in the top 1900 of the first reservoir 1802 is no greater than 164 mm, no greater than 154 mm, no greater than 144 mm, no greater than 134 mm, or no greater than 124 mm. In some embodiments, the width, W3, of the recessed area 1806 in the top 1900 of the first reservoir 1802 is at least 124 mm, at least 134 mm, at least 144 mm, at least 154 mm, or at least 164 mm. In some embodiments, the width, W3, of the recessed area 1806 in the top 1900 of the first reservoir 1802 that allows for culturing cells at suitably-high throughput is in a range of 139 to 149 mm. FIG. 20 shows the recessed area 1806 as having a height (or depth), H8. In some embodiments, the height, H8, of the recessed area 1806 in the top 1900 of the first reservoir 1802 is no greater than 40 mm, no greater than 35 mm, no greater than 30 mm, no greater than 25 mm, or no greater than 20 mm. In some embodiments, the height, H8, of the recessed area 1806 in the top 1900 of the first reservoir 1802 is at least 20 mm, at least 25 mm, at least 30 mm, at least 35 mm, or at least 40 mm. In some embodiments, the height, H8, of the recessed area 1806 in the top 1900 of the first reservoir 1802 that allows for culturing cells at suitably-high throughput is in a range of 25 to 35 mm.

The first reservoir 1802 may include an M×N array of microfluidic channels 1808, with M rows of microfluidic channels 1808 and N columns of microfluidic channels 1808, where M can be any suitable integer and N can be any suitable integer. Accordingly, the first row of microfluidic channels 1808 includes microfluidic channels 1808(1)(1) through 1808(1)(N). A second row of microfluidic channels 1808 includes microfluidic channels 1808(2)(1) through 106(2)(N), and so on and so forth until an M^{th} row of microfluidic channels 1808 that includes microfluidic channels 1808(M)(1) through 106(M)(N). In FIG. 18, only the inlets to the microfluidic channels 1808 are visible. FIGS. 19-26 show further details of example embodiments of the microfluidic channels 1808.

FIG. 21 depicts an example layout of the microfluidic channels 1808 in the form of spiral channels 1908, while FIG. 25 depicts an example layout of the microfluid channels 1808 in the form of serpentine channels 2308. In both of FIGS. 21 and 25, the example M×N array of microfluidic channels 1808 is a 10×10 array of microfluidic channels 1808, where M=10 and N=10. This configuration enables high-throughput, in vitro culturing of three-dimensional (3D) human organ models, such as bone marrow models, in small volumes and for several applications, such as drug toxicity and drug efficacy. This high-throughput system can allow for performing a relatively large amount of experiments, as compared to existing systems, and it allows for doing so in a robust and reliable manner. In the pharmaceutical industry, this allows for realizing the full potential of the in vitro organ models in a drug discovery pipeline. In some embodiments, a 10×10 array of microfluidic channels 1808 is implemented in the first reservoir 1802 with a recessed area 1806 having a width, W3, of 144 mm, a length, L5, of 115 mm, and a height (or depth), H8, of 30 mm. These example dimensions of the recessed area 1806 create a volume of about 500 mL within the first reservoir 1802 to receive fluid media therein.

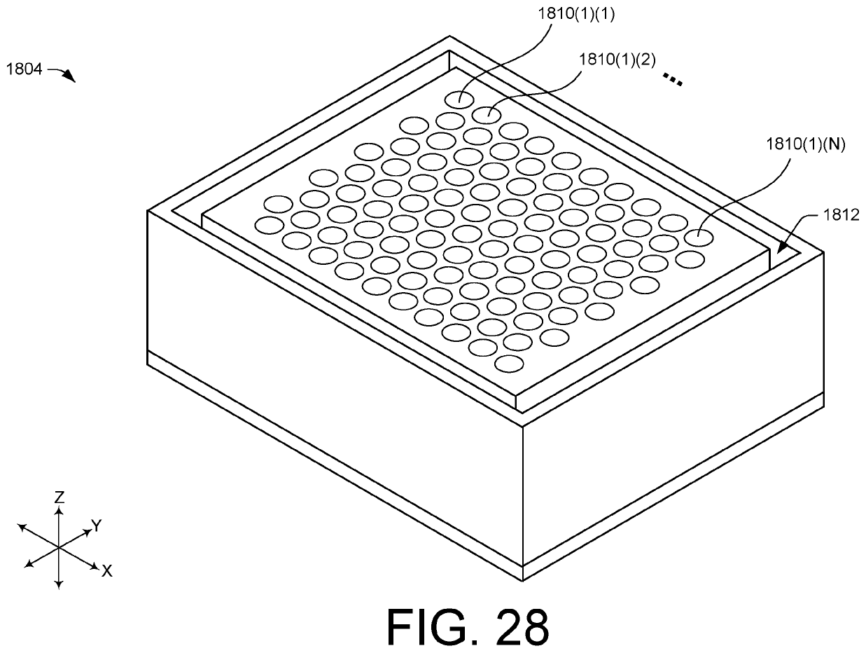

When an array of inserts (See FIG. 27 for an example insert) are installed in the first reservoir 1802, the gravity-based sample box 1800 is configured to culture cells, such as bone marrow cells. For example, the recessed area 1806 at the top 1900 of the first reservoir 1802 may be filled with a fluid media. This fluid media may be a cell culture media, such as media that contains nutrition, oxygen or other gases, or other elements for the maintenance, growth, or testing of cells or agents (e.g., cells in hydrogel). Media flow is gated by the microfluidic channels 1808 and the media passes through the inserts installed in the first reservoir 1802. Within the inserts, the media may flow over an endothelial monolayer to indirectly sustain a hydrogel containing cells (e.g., a mineralized niche, such as shards of mineralized collagen embedded in a cell-laden hydrogel). Media that egresses from the first reservoir 1802 may be collected in an array of collection wells 1810 defined in the second reservoir 1804. Notably, the isolated nature of the microfluidic channels 1808 and the collection wells 1810, as well as the alignment therebetween allow for conducting separate, isolated tests, where an individual pair of a microfluidic channel 1808 and a collection well 1810 positioned underneath the channel 1808 acts as a separate “test lane” that is not cross contaminated by other test lanes of the system 1800. In other words, the fluid media that passes through a specific microfluidic channel 1808 can be collected and maintained in an individual collection well 1810 of the second reservoir. These individual collection wells 1810 of the second reservoir can be seen in better detail in FIGS. 28-31. After fluid media that has passed through the array of microfluidic channels 1808 and has been collected in the array of collection wells 1810, an operator may sample the collected media (e.g., using a pipette, such as a standard 1 mL pipette) and may analyze the sample to determine analysis results, as described herein. The second reservoir 1804 is also shown as having a recessed section 1812 at a periphery of the second reservoir to allow the first reservoir 1802 to interlock with the second reservoir 1804 and prevent leakage during use.

The first reservoir 1802 and/or the second reservoir 1804 may be made of any suitable material, combination of materials, or composite materials. For example, the reservoir(s) 1802/1804 can be made of any of the polymer/plastic materials described herein, such as a laboratory-grade plastic including, without limitation, polycarbonate plastic, polystyrene, polyethylene, PET, and the like. It is to be appreciated that the reservoir(s) 1802/1804 can be made of any other suitable rigid or semi-rigid material that is suitable for cell culturing.

In some embodiments, the reservoir(s) 1802/1804 may be manufactured using an injection molding technique, or an extrusion technique, the processes for which are apparent to a person having ordinary skill in the art. Other manufacturing techniques that may be used to manufacture the reservoir(s) 1802/1804 include machining a material into the shape of the reservoir(s) 1802/1804, or into component parts (e.g., substrates) of the reservoir(s) 1802/1804 that are attached together during manufacture using any suitable fastening means, such as screws, pins, joints, adhesives, or the like. Any other subtractive manufacturing techniques can be used besides machining. Additionally, additive manufacturing techniques, such as 3D printing, can be used to manufacture the reservoir(s) 1802/1804.

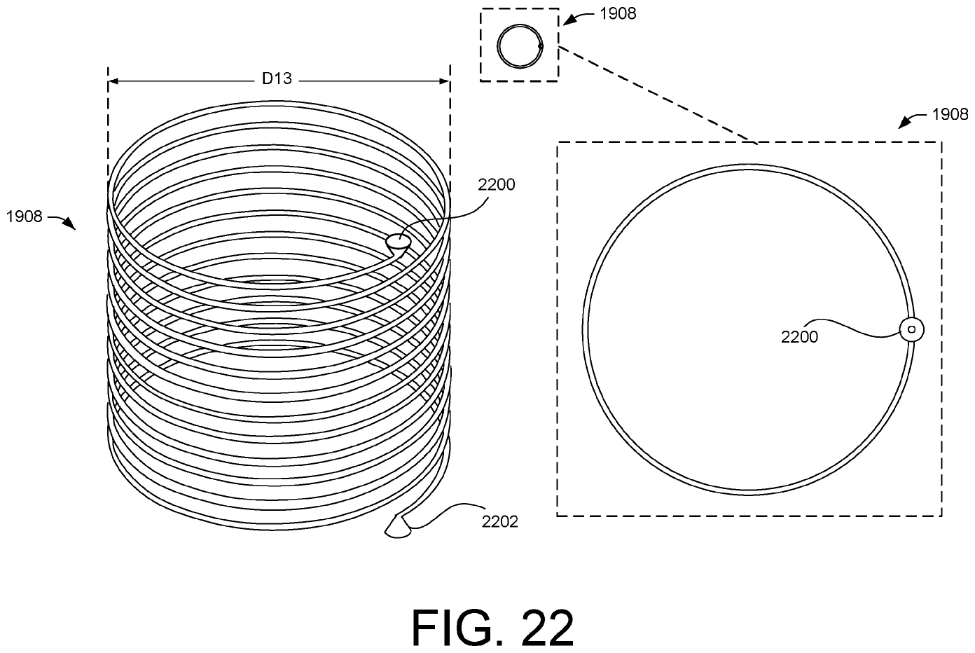

The microfluidic channels 1808 may be implemented in various ways. One example way is shown in FIGS. 19-22, where the microfluidic channels 1808 are shown in the form of spiral channels 1908 that are formed in the first reservoir 1802 and positioned underneath the recessed area 1806. The spiral channels 1908 create a series of microfluidic pathways that are isolated from one another. Referring to FIG. 22, there is illustrated a perspective view and a top view of an example spiral channel 1908 that may be defined in the first reservoir 1802. The spiral channel 1908 includes an inlet 2200 and an outlet 2202. The inlet 2200 is positioned at the bottom of the recessed area 1806 in the first reservoir 1802 to receive fluid media within the spiral channel 1908. Fluid media is influenced to flow into the inlet 2200 of the spiral channel 1908 by the force of gravity. The outlet 2202 of the spiral channel 1908 is positioned at a top of an additional recessed area 1904 defined in the bottom 1902 of the first reservoir 1802. The additional recessed area 1904 forms a lip or rim around a periphery of the bottom 1902 of the first reservoir 1802, the function of which can be to interlock with the second reservoir 1804 by being inserted into the recessed section 1812 defined in the top of the second reservoir 1804. In some embodiments, a height (or depth), H9, of the additional recessed area 1904 is about 3 mm.

FIG. 20 shows the spiral channel 1908 as having a length, L6, while FIG. 22 shows the spiral channel 1908 as having an overall diameter, D13. In some embodiments, the length, L6, of the spiral channel 1908 is no greater than 11 mm, no greater than 10 mm, no greater than 9 mm, no greater than 8 mm, or no greater than 7 mm. In some embodiments, the length, L6, of the spiral channel 1908 is at least 7 mm, at least 8 mm, at least 9 mm, at least 10 mm, or at least 11 mm. In some embodiments, the length, L6, of the spiral channel 1908 that allows for an optimized flow rate of fluid media is in a range of 8 to 10 mm. In some embodiments, the overall diameter, D13, of the spiral channel 1908 is no greater than 11 mm, no greater than 10 mm, no greater than 9 mm, no greater than 8 mm, or no greater than 7 mm. In some embodiments, the overall diameter, D13, of the spiral channel 1908 is at least 7 mm, at least 8 mm, at least 9 mm, at least 10 mm, or at least 11 mm. In some embodiments, the overall diameter, D13, of the spiral channel 1908 that allows for an optimized flow rate of fluid media is in a range of 8 to 10 mm. Other parameters of the spiral channel 1908 may include, without limitation, a pitch of the spiral channel 1908, a cross-sectional shape of the channel pathway, a distance traveled through the spiral channel 1908, etc. For example, the cross-sectional shape of the spiral channel pathway can be any suitable shape, such as circular, square-shaped, rectangular, triangular, or any suitable 2D polygonal shape. In some embodiments, the cross-sectional shape of the spiral channel pathway is a square-shape having a cross-sectional area of about 0.0225 mm². The distance that fluid media travels through the spiral channel 1908 from the inlet 2200 to the outlet 2202 may be about 300 mm.

FIG. 21 further shows an example layout of a 10×10 array of spiral channels 1908 in the first reservoir 1802A. As depicted in FIG. 21, every other row of spiral channels 1908 is offset laterally from an adjacent row by roughly have the diameter D13 of the spiral channel 1908. This staggered arrangement may allow for a higher-density layout of spiral channels 1908, as compared to an arrangement where the columns are vertically-aligned across all rows of spiral channels 1908, which, in turn, may allow for a smaller overall system 1800.

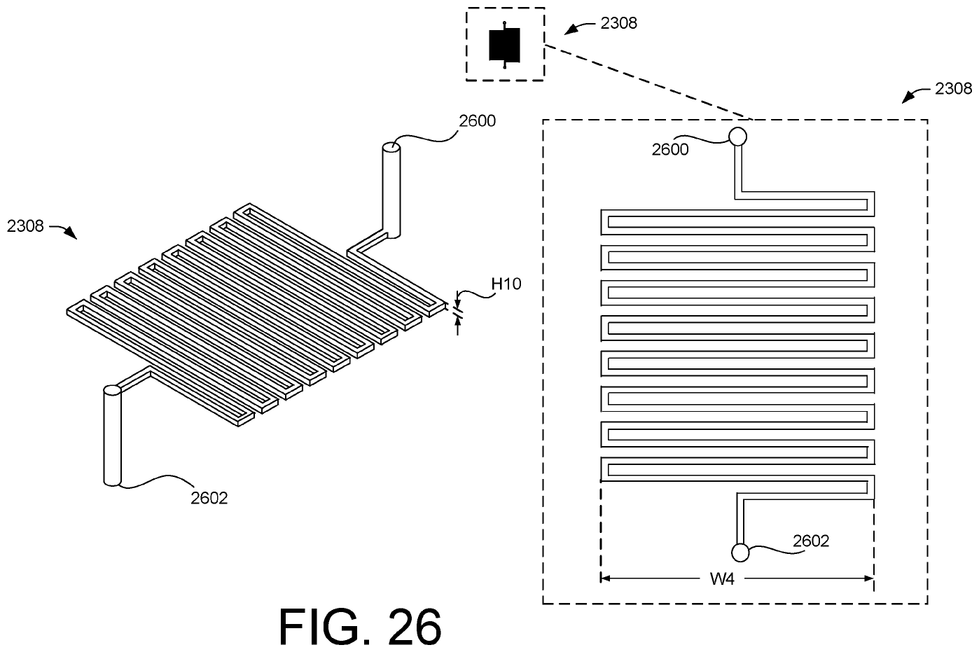

Another example way of implementing the microfluidic channels 1808 is shown in FIGS. 23-26, where the microfluidic channels 1808 are shown in the form of serpentine channels 2308 that are formed in the first reservoir 1802 and positioned underneath the recessed area 1806. The serpentine channels 2308 create a series of microfluidic pathways that are isolated from one another. Referring to FIG. 26, there is illustrated a perspective view and a top view of an example serpentine channel 2308 that may be defined in the first reservoir 1802. The serpentine channel 2308 includes an inlet 2600 and an outlet 2602. The inlet 2600 is positioned at the bottom of the recessed area 1806 in the first reservoir 1802 to receive fluid media within the serpentine channel 2308. Fluid media is influenced to flow into the inlet 2600 of the serpentine channel 2308 by the force of gravity. The outlet 2602 of the serpentine channel 2308 is positioned at a top of the additional recessed area 1904 defined in the bottom 1902 of the first reservoir 1802. As shown in FIG. 26, the serpentine channel 2308 is a generally flat, planer channel that “snakes” along a serpentine path that is horizontally-oriented.

The serpentine channel 2308 may have an overall length, L6, between the inlet 2600 and the outlet 2602 that is the same, or similar, to the length of the spiral channel 1908 described herein. FIG. 26 shows the serpentine channel 2308 as having an overall width, W4. In some embodiments, the overall width, W4, of the serpentine channel 2308 is no greater than 8 mm, no greater than 7 mm, no greater than 6 mm, no greater than 5 mm, or no greater than 4 mm. In some embodiments, the overall width, W4, of the serpentine channel 2308 is at least 4 mm, at least 5 mm, at least 6 mm, at least 7 mm, or at least 8 mm. FIG. 26 also shows the serpentine channel 2308 as having channel height, H10. In some embodiments, the channel height, H10, of the serpentine channel 2308 is no greater than 0.25 mm, no greater than 0.15 mm, or no greater than 0.5 mm. In some embodiments, the channel height, H10, of the serpentine channel 2308 is at least 0.5 mm, at least 0.15 mm, or at least 0.25 mm. Other parameters of the serpentine channel 2308 may include, without limitation, a pitch of the serpentine channel 2308, a cross-sectional shape of the channel pathway, a distance traveled through the spiral channel 2308, a number of serpentine lanes, etc. For example, the cross-sectional shape of the serpentine channel pathway can be any suitable shape, such as circular, square-shaped, rectangular, triangular, or any suitable 2D polygonal shape. In some embodiments, the cross-sectional shape of the spiral channel pathway is a square-shape having a cross-sectional area of about 0.0225 mm². The distance that fluid media travels through the serpentine channel 2308 from the inlet 2600 to the outlet 2602 may be about 300 mm.

FIG. 25 further shows an example layout of a 10×10 array of serpentine channels 2308 in the first reservoir 1802B. As depicted in FIG. 25, every other row of serpentine channels 2308 is offset laterally from an adjacent row by roughly have the width, W4, of the serpentine channel 2308. This staggered arrangement may allow for a higher-density layout of serpentine channels 2308, as compared to an arrangement where the columns are vertically-aligned across all rows of serpentine channels 2308, which, in turn, may allow for a smaller overall system 1800.

In any of the configurations described herein, the microfluidic channels 1808 (e.g., 1908 and 2308) have parameters, as described above, which can be modified or tuned to specific needs. For example, a microfluidic channel 1808 having a particular cross-sectional area may be designed for a certain type of test. Both spiral and serpentine configuration of the microfluidic channels 1808 were modeled for fluid flow properties and in the case of the serpentine channels 2308, the ability to control diffusion properties if used in contact with an endothelial monolayer of an insert coupled thereto was modeled. The configurations accommodate a max flow rate of 1 μL/min per microfluidic channel 1808 and a flow rate of 0.25 μL/min was reached after 132 hours as listed in Table 1.

TABLE 1

| Channel | Reservoir | | | Time (hr) to |
|---|---|---|---|---|
| x-section | height, | Length | Qmax | reach |
| dimension | H8, (mm) | (cm) | (μL/min) | 0.25 μL/min |
| 150 | 35 | 30 | 1.06 | 132 |
| 150 | 25 | 20 | 1.14 | 92 |
| 150 | 15 | 14 | 1.01 | 57 |
| 200 | 35 | 100 | 1.01 | 134 |
| 200 | 25 | 70 | 1.03 | 95 |
| 200 | 15 | 40 | 1.08 | 56 |

Flow rate in this system 1800 is maintained via the media level in the first reservoir 1802. Using a reservoir size of L5=144 mm, W3=115 mm, and H8=30 mm, and maximum flowrate, Qmax, the system 1800 can operate for approximately 83 hours. Diffusion rate and additional modeling are specified in Table 2.

TABLE 2

| # of | Starting flowrate | Time (hr) to reach |
|---|---|---|
| paths | (μL/min) | 0.25 μL/min |
| 11 | 1.19 | 18.7 |
| 12 | 1.11 | 20.1 |
| 13 | 1.03 | 21.5 |
| 14 | 0.96 | 23 |
| 15 | 0.91 | 24 |

FIG. 27 illustrates views of an example insert 2700 that is configured to be coupled to an inlet 2200/2600 or an outlet 2202/2602 of a microfluidic channel 1808. For example, the insert 2700 includes an inlet 2702 and an outlet 2704. The outlet 2704 of the insert 2700 may be coupled to the inlet 2200/2600 of a microfluidic channel 1808 by any suitable means, such as by being press fit onto the inlet 2200/2600, threaded onto the inlet 2200/2600, snapped onto the inlet 2200/2600, or the like. In this case, the inlet 2200/2600 may be raised from the bottom of the recessed area 1806 to accommodate such a coupling. Furthermore, the inlet 2702 of the insert 2700 may be coupled to the outlet 2202/2602 of the microfluidic channel 1808 by any suitable means, such as by being press fit onto the outlet 2202/2602, threaded onto the outlet 2202/2602, snapped onto the outlet 2202/2602, or the like. In this case, the outlet 2202/2602 may be raised from the top of the additional recessed area 1904 to accommodate such a coupling.

The inlet 2702 of the insert 2700 may be positioned at a top of the insert 2700 and on a first side of the insert 2700, while the outlet 2704 of the insert 2700 may be positioned at a bottom of the insert 2700 and on a second side of the insert 2700 opposite the first side, thereby allowing gravity to influence the flow of fluid media from the inlet 2702 to the outlet 2704 of the insert 2700 and allowing the fluid media to pass through the insert 2700 in a transverse/horizontal direction. FIG. 27 illustrates a cross-sectional view of the insert 2700 taken along section-line C-C. Defined in the insert 700 is a media channel 2706 for fluid media to pass through the insert 2700, as well as a hydrogel chamber 2708 that houses hydrogel 700 containing cells (or an ECM 700). The hydrogel chamber 2708 is shown as being positioned underneath the media channel 2706.

As further depicted in FIG. 6, a permeable barrier 2710 (sometimes referred to herein as a “permeable membrane 2710,” a “porous membrane 2710,” or “permeable layer 2710”) is interposed between the media channel 2706 and the hydrogel chamber 2708. Accordingly, the hydrogel chamber 2708 may include a top end that is covered with the permeable barrier 2710. For example, the permeable barrier 2710 may be affixed (e.g., bonded) to the top end of the hydrogel chamber 2708. In some embodiments, the permeable barrier 2710 may include PDMS or polyethylene terephthalate (PETE) capable of allowing passage of desired media and contents, such as nutrients, metabolites, waste, etc. One commercially available membrane for this purpose is the PE1009030 10.0 micron, 90 mm polyester (PETE) membrane filters available from Sterlitech Corporation (Kent, Wash., USA). In some embodiments, the permeable barrier 2710 may be greater than the cross-sectional area of the hydrogel chamber 2708, such as slightly greater than to cover the top end of the hydrogel chamber 2708 without waste of material. In some embodiments, the permeable barrier 2710 can span a larger area, such as an entirety of the insert 2700. As depicted in FIG. 27, the hydrogel chamber 2708 is positioned underneath the permeable barrier 2710, and the media channel 2706 is positioned atop the permeable barrier 2710.

In some embodiments, the hydrogel 700 (sometimes referred to herein as an “extracellular matrix (ECM) 700”) within the hydrogel chamber 2708 may be formed by cutting or breaking material (or elements) representing an endosteal niche of a bone marrow model into shards, and embedding or mixing the shards throughout a cell-laden hydrogel that represents a vascular niche of the bone marrow model. In some embodiments, the material representing the endosteal niche may include mineralized collagen. In some embodiments, the shards of mineralized collagen may encapsulate or contain cells found in the natural endosteal niche of normal bone, including, without limitation, osteoblasts, osteoprogenitors, and/or osteochondroprogenitors. In some embodiments, the cell-laden hydrogel in which the shards are embedded may itself encapsulate or contain cells found in the natural vascular (or stem cell, or hematopoietic) niche of normal bone, including, without limitation, hematopoietic stem cells (HSCs), long-term hematopoietic stem cells (LT-HSCs), short-term hematopoietic stem cells (ST-HSCs), multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), and/or other specialized marrow stromal populations such as CXCL12-abundant reticular (CAR) cells. In some embodiments, endothelial cells, such as a Human Umbilical Vein Endothelial Cells (HUVEC) monolayer, may be adhered to top face or side of the porous membrane 2710. Accordingly, the permeable barrier 2710 may sometimes be referred to as an “endothelial monolayer 2710”.

An array of inserts 2700 may be installed in the first reservoir 1802 at the bottom of the recessed area 1806 and/or at the top of the additional recessed area 1904. For example, the outlet 2704 of the insert 2700 may be coupled to an inlet 2200/2600 of a microfluidic channel 1808. Additionally, or alternatively, the inlet 2702 of another insert 2700 may be coupled to an outlet 2202/2602 of another microfluidic channel 1808. In this manner, when the first reservoir 1802 is filled with fluid media 702, such as by pouring fluid media 702 from a source container into the recessed area 1806 of the first reservoir 1802, the fluid media 702 flows through the microfluidic channels 1808 and through the inserts 2700. Depending on where the inserts 2700 are installed, the fluid media 702 may flow through the microfluidic channels 1808 before or after flowing through the inserts 2700. When the fluid media 702 flows through a given insert 2700, the fluid media 702 remains generally segregated from the hydrogel 700 in the hydrogel chamber 2708 by virtue of the permeable barrier 2710, except for the passage of nutrients, metabolites, waste, etc. through the permeable barrier 2710. Meanwhile, the fluid media 702 flows over the permeable barrier 2710 and egresses from the insert 2700 via the outlet 2704 of the insert 2700.

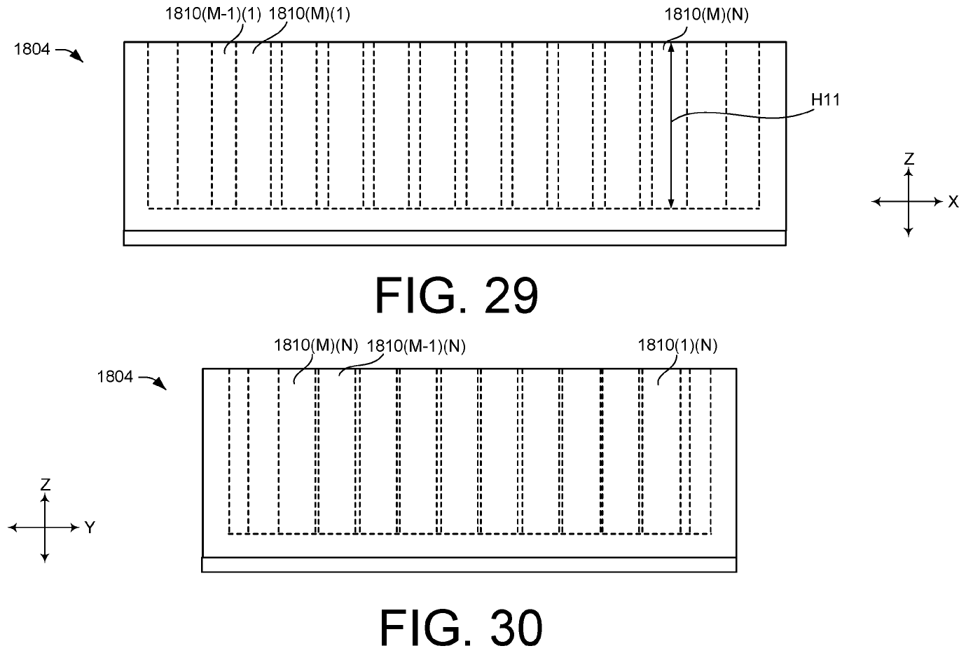

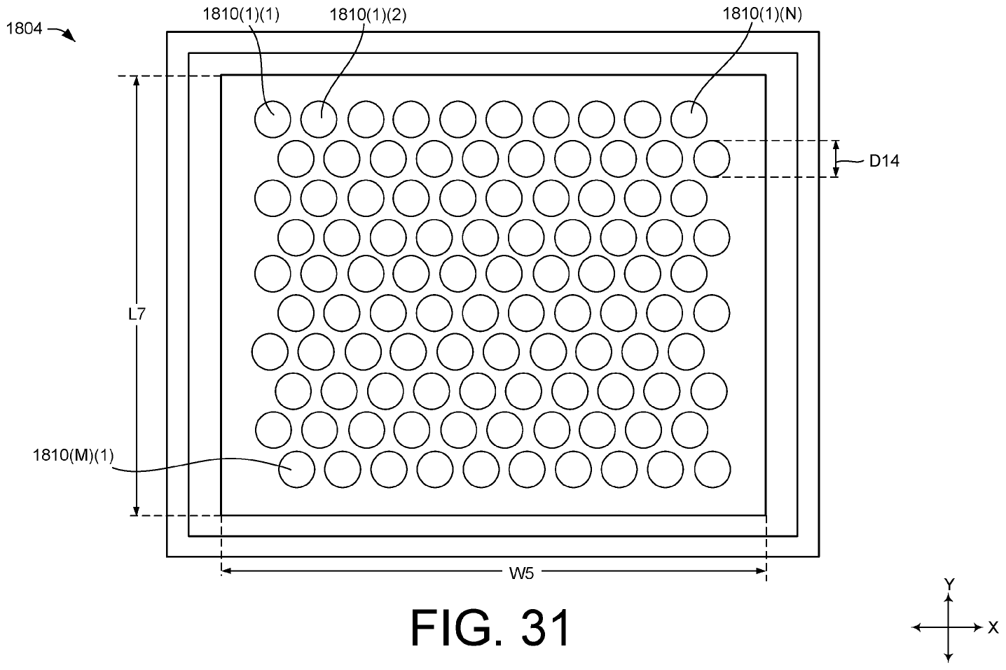

FIG. 28 illustrates the second reservoir 1804 of the system 1800. The second reservoir 1804 is shown from a perspective view in FIG. 28. FIG. 29 illustrates a front view of the example second reservoir 1804 of FIG. 28, FIG. 30 illustrates a side view of the example second reservoir 1804 of FIG. 28, and FIG. 31 illustrates a top view of the example second reservoir 1804 of FIG. 28. The second reservoir 1804, also referred to herein as an “outlet reservoir 1804,” when coupled to the first reservoir 1802, may be configured to collect, in the array of collection wells 1810, fluid media that has passed through the first reservoir 1802 by passing through the array of microfluidic channels 1808 and through the array of inserts 2700.

As shown in FIG. 28, the second reservoir 1804 may include an M×N array of collection wells 1810, with M rows of collection wells 1810 and N columns of collection wells 1810, where M can be any suitable integer and N can be any suitable integer. The example M×N array of collection wells 1810 shown in FIG. 28 is a 10×10 array of collection wells 1810 where M=10 and N=10. With a first reservoir 1802 having a 10×10 array of microfluidic channels 1808, this provides a one-to-one correspondence of microfluidic channels 1808 to collection wells 1810 due to the grid-like arrangement of both arrays in the respective reservoirs.

The M×N array of collection wells 1810 are positioned underneath the outlets 2202/2602 of the M×N array of microfluidic channels 1808 at the bottom 1902 of the first reservoir 1802 in order to collect fluid media egressing from the first reservoir 1802. As mentioned, when inserts 2700 are coupled to the bottom of the first reservoir 1802, the collection wells 1810 may collect fluid media egressing from the outlets 2704 of the inserts 2700.

FIG. 31 shows an individual collection well 1810 as having a diameter, D14. In some embodiments, the diameter, D14, of an individual collection well 1810 is no greater than 10.5 mm, no greater than 9.5 mm, no greater than 8.5 mm, no greater than 7.5 mm, or no greater than 6.5 mm. In some embodiments, the diameter, D14, of an individual collection well 1810 is at least 6.5 mm, at least 7.5 mm, at least 8.5 mm, at least 9.5 mm, or at least 10.5 mm. FIG. 29 shows an individual collection well 1810 as having a height (or depth), H11. In some embodiments, the height, H11, of an individual collection well 1810 is no greater than 70 mm, no greater than 60 mm, no greater than 50 mm, no greater than 40 mm, or no greater than 30 mm. In some embodiments, the height, H11, of an individual collection well 1810 is at least 30 mm, at least 40 mm, at least 50 mm, at least 60 mm, or at least 70 mm. FIG. 31 also shows the portion of the second reservoir 1804 in which the collection wells 1810 are defined as having a length, L7, and a width, W5. In some embodiments, the length, L7, of the area of collection wells 1810 is no greater than 125 mm, no greater than 115 mm, no greater than 105 mm, no greater than 95 mm, or no greater than 85 mm. In some embodiments, the length, L7, of the area of collection wells 1810 is at least 85 mm, at least 95 mm, at least 105 mm, at least 115 mm, or at least 125 mm. In some embodiments, the width, W5, of the area of collection wells 1810 is no greater than 150 mm, no greater than 140 mm, no greater than 130 mm, no greater than 120 mm, or no greater than 110 mm. In some embodiments, the width, W5, of the area of collection wells 1810 is at least 110 mm, at least 120 mm, at least 130 mm, at least 140 mm, or at least 150 mm. In some embodiments, the layout of the collection wells 1810 is configured to accommodate a 1 mL multi-channel pipette pattern, and a 1 mL pipette tip may fit within an individual collection well 1810.

The individual collection wells 1810 may have a volume of approximately 2.8 mL. Because each collection well 1810 collects fluid media that passes through a single microfluidic channel 1808 and a single insert 2700, the system 1800 including the second reservoir 1804 allows for monitoring each “test lane” as a replicate in the system 1800.

The system 1800 constitutes a microfluidic design that allows for easy use and a high replicate number of experimental tests, and is therefore a high-throughput system. The system 1800 can be used for complex 3D and 2D tissue culture constructs. The microfluidics miniaturizes the system for testing tissue constructs that mimic biological systems. In some embodiments, the system 1800 can be placed in an incubator and left for a period of time with the fluid media 702 passing through the inserts 2700 and collecting in the collection wells 1810. An operator may periodically take samples of the contents (e.g., the fluid media 702 collected in the collection wells 1810 and/or the hydrogel 700 in the inserts 2700), may analyze the samples, and may determine analysis results (e.g., to determine how a drug is interacting with the cells). Each vertically-oriented set of features (e.g., an insert 270 containing hydrogel 700 underneath a permeable membrane 2710, a microfluidic channel 1808 (e.g., a spiral channel 1908, a serpentine channel 2308, etc.), and a collection well 1810 collecting the fluid media that drains through the first reservoir 1802) may represent an isolated organ reactor (or “bioreactor”). The system 1800 may also allow for directional flow and non-recycled media usage. The system 1800 may allow for the testing of proteins and pharmaceuticals in bioinspired tissue constructs. The use of gravity as a “pump” mechanism allows for a simplified fluidics system, expanding the accessibility to many more non-specialized tissue engineering and biology labs. The system 1800 provides non-expert labs with access to microfluidics without the high cost of specialized equipment.

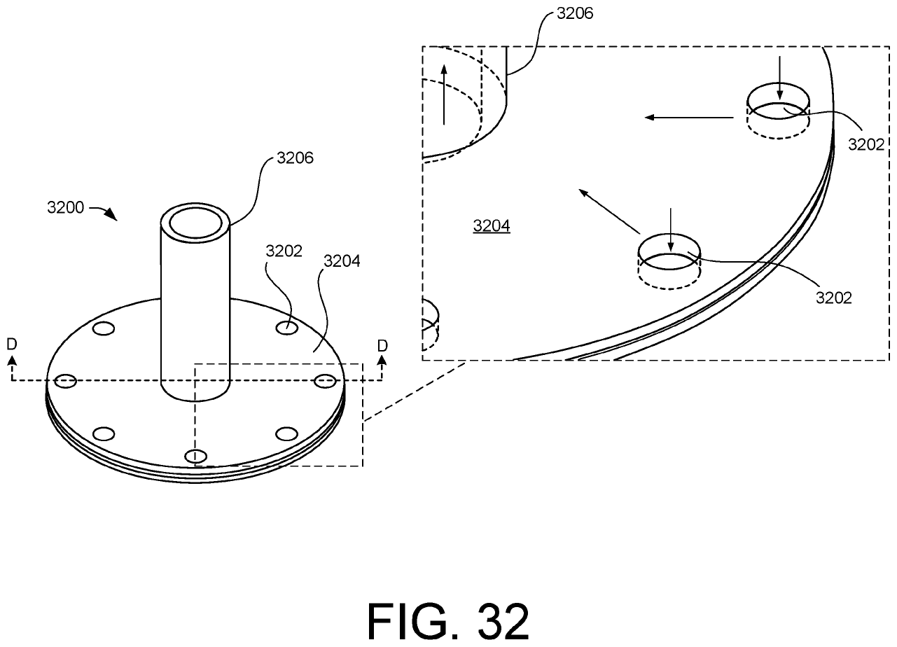

FIG. 32 illustrates a perspective view of vacuum insert 3200, according to embodiments disclosed herein. This vacuum insert 320 is a microfluidic design that makes use of traditional well plates and vacuum lines available in many tissue culture facilities. This vacuum insert 3200 is configured to be inserted into, or placed inside, an individual well of a 24 well plate, and in example. In this scenario, 24 vacuum inserts 3200 can be inserted into the 24 media wells to create multiple isolated chambers in which a mineralized hydrogel niche and endothelial monolayer can be cultured under one or more flow conditions. The vacuum insert 3200, when inserted into a media well of a well plate, is configured to create a separation between a hydrogel containing cells at the bottom of the well plate and the media (e.g., fluid, culture media) at the top of the well plate. Media is then drawn, via vacuum suction, through the vacuum insert 3200. For example, fluid media is drawn into one or more inlets 3202 defined in the top of an annular base 3204 of the vacuum insert 3200, and the fluid media flows through a media channel within the annular base 3204, passing over a permeable barrier (e.g., an endothelial monolayer), and egressing from the vacuum insert by traveling upward through a central tube 3206 coupled to the annular base 3204. The central tube 3206 may be coupled to the annular base 3204 at the center of the annular base 3204 and may extend orthogonally from the top of the annular base 3204. Each media well within well plate that contains a vacuum insert 320 can be aspirated separately, or a plurality of media wells can be aspirated collectively, depending on testing objectives. The vacuum insert 3200 can interface with tubing at a top end of the central tube 3206. For example, a vacuum line of a vacuum source (e.g., a pump) may be connected to the top end of the central tube 3206. Additionally, or alternatively, a lid designed to connect with multiple vacuum inserts 3200 can be placed over an entire well plate to provide suction (due to operation of a vacuum source coupled to the lid) across a plurality of media wells. The zoomed-in view of the portion of the vacuum insert 3200 shown in FIG. 32 illustrates, via arrows, a direction of fluid flow, where fluid media enters the vacuum insert 3200 via the inlets 3202 defined in the top of the annular base 3204 and travels through the interior of the annular base 3204 in a radially-inward direction towards the central tube 3206, and then up through the central tube 3206. A plurality of inlets 3202 may be spaced equidistantly around the periphery of the annular base 3204.

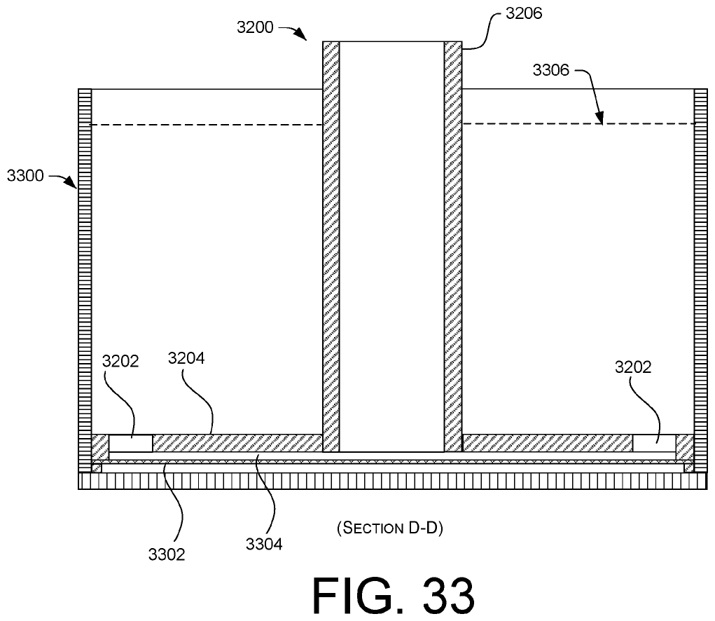

FIG. 33 illustrates a cross-sectional view of the vacuum insert 3200 when inserted into, or placed inside, a media well 3300 with the annular base 3204 at a bottom of the media well 3300. The central tube 3206 may couple to the annular base 3204 in any suitable fashion, such as a threaded engagement where the central tube 3206 includes external threads at a bottom end that are screwed into internal threads of a centrally-located bore in the top of the annular base 3204. As shown in FIG. 33, the annular base 3204 may have a permeable barrier 3302 (e.g., an endothelial monolayer) that spans the area of the annular base 3204, and that is positioned underneath a media channel 3304 such that fluid media flows over the permeable barrier 3302 when passing through the annular base 3204. The media channel 3304 may be in fluid communication with the inlets 3202. A hydrogel containing cells, such as the hydrogel 700 discussed herein, may be placed at a bottom of the media well 3300, and the vacuum insert 3200 may be placed atop the hydrogel layer such that the annular base 3204 covers the hydrogel layer. In this configuration, the annular base 3204 may have a recessed area in the bottom of the annular base 3204 to accommodate the hydrogel underneath the permeable barrier 3302. Alternatively, the annular base 3204 may have defined therein a hydrogel chamber that can be pre-loaded with hydrogel, similar to the hydrogel chambers 200 described herein. With a hydrogel chamber, the annular base 3204 may have a bottom that encloses the hydrogel chamber. In either case, the permeable barrier 3302 may separate the hydrogel from the fluid media by being interposed between the media channel 3304 and the hydrogel. FIG. 33 depicts a media level 3306 to indicate that the media well 3300 may be filled with fluid media (e.g., culture media) and the fluid media may generally reside above the annular base 3204 of the vacuum insert 3200. When a vacuum source, such as a pump, is operated, the fluid media may flow into the vacuum insert 3200 via the inlets 3202, travel through the annular base 3204 passing over the permeable barrier 3302, and up through the central tube 3206, which is in fluid communication with the media channel 3304. This allows for cell culturing, as described herein.

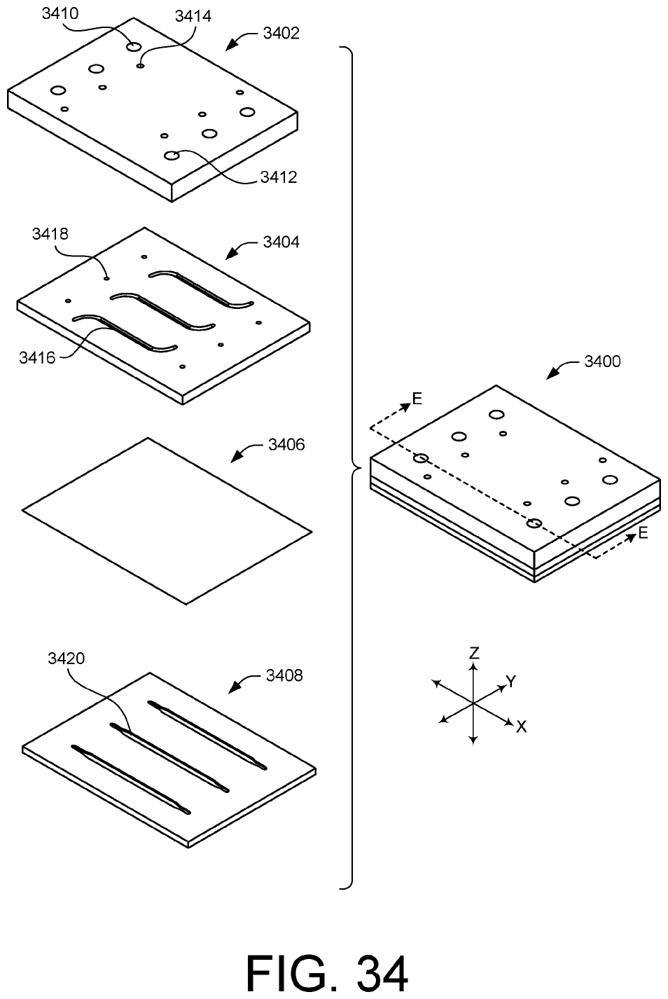

FIG. 34 illustrates a perspective exploded view and a perspective assembled view of an example microfluidic chip 3400 (or device 3400), according to embodiments disclosed herein. FIG. 35 depicts a cross-section of the chip 3400 taken along section-line E-E. The microfluidic chip 3400 may comprise multiple stacked layers or substrates. For example, the microfluidic chip 3400 may comprise a first substrate 3402 (sometimes referred to herein as a “top substrate 3402”, or a “connector substrate 3402”). The first substrate 3402 may be disposed atop a second substrate 3404 (sometimes referred to herein as an “intermediate substrate 3404”, or a “cell+hydrogel substrate 3404”). Accordingly, the second substrate 3404 may be disposed underneath the first substrate 3402. The second substrate 3404 may also be disposed atop a permeable barrier 3406 (or “permeable membrane 3406”, “porous membrane 3406”, etc.). Accordingly, the permeable barrier 3406 may be disposed underneath the second substrate 3404. The permeable barrier 3406 may also be disposed atop a third substrate 3408 (sometimes referred to herein as a “bottom substrate 3408”, or a “media substrate 3408”). Accordingly, the third substrate 3408 may be disposed underneath the permeable barrier 3406, the second substrate 3404 may be interposed between the first substrate 3402 and the third substrate 3408, and the permeable barrier 3406 may be interposed between the second substrate 3404 and the third substrate 3408.

The first substrate 3402 may include one or more inlets 3410, such as three inlets 3410 defined in a top of the connector substrate 3402. Multiple inlets 3410 may be positioned at, or near, a periphery of the connector substrate 3402 and spaced along a side edge of the connector substrate 3402. The connector substrate 3402 may further include one or more outlets 3412, such as three outlets 3412 defined in the top of the connector substrate 3402. Multiple outlets 3412 may be positioned at, or near, a periphery of the connector substrate 3402 and spaced along a second side edge of the connector substrate 3402 that is opposite the first side edge along which the inlets 3410 are spaced. An individual inlet 3410 may be configured to allow fluid media to ingress into the device 3400, and an individual outlet 3412 may be configured to allow the fluid media to egress from the device 3400.

The first substrate 3402 may further include one or more loading ports 3414, such as six loading ports 3414 defined in the top of the connector substrate 3402. FIG. 34 shows these loading ports 3414 as being offset (in the Y-direction) from the inlets 3410 and the outlets 3412. In some embodiments, three loading ports 3414 are to one side of a center of the connector substrate 3402 but farther from a periphery than the inlets 3410, and another three loading ports 3414 are to the other side of the center of the connector substrate 3402 but farther from a periphery than the outlets 3412. An individual loading port 3414 may be configured to receive a hydrogel, such as the hydrogel 700 containing cells, as described herein.

The second substrate 3404 may have defined therein one or more hydrogel channels 3416 configured to receive the hydrogel 700. The second substrate 3404 is shown as having three hydrogel channels 3416 defined in a top of the second substrate 3404, spanning a center of the second substrate 3404, and oriented horizontally on the second substrate 3404. A center portion of an individual hydrogel channel 3416 may be straight, and one or more peripheral portions of the hydrogel channel 3416 may be curved. FIG. 34 depicts both peripheral portions of each hydrogel channel 3416 being curved, and that a first peripheral portion of the channel 3416 curves in a first direction while a second peripheral portion of the channel 3416 curves in a second direction opposite the first direction. In this manner, the ends of the hydrogel channels 3416 may be vertically-aligned with, and disposed underneath, a respective loading port 3414 defined in the first substrate 3402. Accordingly, the hydrogel channels 3416 are in fluid communication with the loading ports 3414 positioned above the channels 3416, and hydrogel can be loaded into the hydrogel channels 3414 via the loading ports 3414. Having multiple (e.g., three) separate hydrogel channels 3414 allows for culturing cells in multiple isolated environments on the same chip 3400.

The second substrate 3404 may further have defined therein one or more through-holes 3418. FIG. 34 depicts six through-holes 3418, three of which are vertically-aligned with the inlets 3410 of the connector substrate 3402, and the other three of which are vertically-aligned with the outlets 3412 of the connector substrate 3402. In this manner, fluid media received via the inlets 3410 may pass through the through-holes 3418 to the layers below the second substrate 3404, and fluid media may subsequently pass through the through-holes 3418 in the opposite side of the second substrate 3404 to egress from the chip 3400 via the outlets 3412 in the connector substrate 3402.

The third substrate 3408 at the bottom of the chip 3400 may include one or more media channels 3420, such as three media channels 3420. The media channels 3420 may span a center of the third substrate 3408 and may be oriented horizontally on the third substrate 3408. The ends of the media channels 3420 may be vertically-aligned with, and disposed underneath, the through-holes 3418 in the second substrate 3404. In this manner, fluid media flowing into the chip 3400 via the inlets 3410 may pass through the through-holes 3418 and may be received in the media channels 3420. When fluid media egresses from the chip 3400, the fluid media may travel from the media channels 3420, through the through-holes 3418 on the opposite side of the chip 3400, and may exit via the outlets 3412 of the chip 3400. This configuration allows for cell culturing in flow conditions.

Implementations of Engineered Bone Marrow:

Existing bone marrow models are based on overly simplified versions of the marrow structure, composition and function. For example, previous work has shown that a bone ossicle implanted into immune compromised mice can support AML cells (Reinisch et al., Nat. Med. 22(7):812-821, 2016), but it does not fully replicate the bone marrow niche.

In the native marrow, the Endosteal niche interfaces with the Vascular niche, providing fundamental sources of instructive signals that maintain and regulate the marrow. The models provided herein recapitulate this interface. There are multiple ways of implementing the endosteal and vascular niches including, but not limited to, an in vitro model with or without flow or with or without fluidic flow, microfluidic chips as well as in vivo models.

The biomimetic bone marrow also can be implemented via different geometries including but not limited to ring, shard, and disk models. In various embodiments, the biomimetic bone marrow can be implanted in vivo or be culture in vitro.

Static Models (In Vitro):

Ring model: Here the endosteal and vascular niches are recreated in a ring-like geometry. The endosteal niche is recreated using the protocol described herein. This can be with different types of hydrogels, with and/or without cells.

In one example, the protocol involves the encapsulation of mature bone cells within a 3D collagenous matrix and subsequent nanoscale mineralization of the matrix via a protein analogue guided mechanism. These non-collagenous protein analogues have the ability to precisely sequester Ca²⁺ and PO₄\³⁻ ions to form a heavily mineralized matrix. Hence through this method, a synthetic 3D construct emulating the cellular, organic and mineral phase of native bone can be generated. The overall process of developing the engineered bone construct can be completed within 7 days, as opposed to the conventional osteoinduction medium (L-ascorbic acid, Dexamethasone, β-glycerolphosphate) which takes a minimum of 21 days for the cells to synthesize and secrete minerals. Following biomimetic mineralization, an inner core is made within the 3D mineralized scaffold to create a bone marrow hematopoietic compartment. A step-by-step illustration of a ring model preparation is illustrated in FIG. 5.

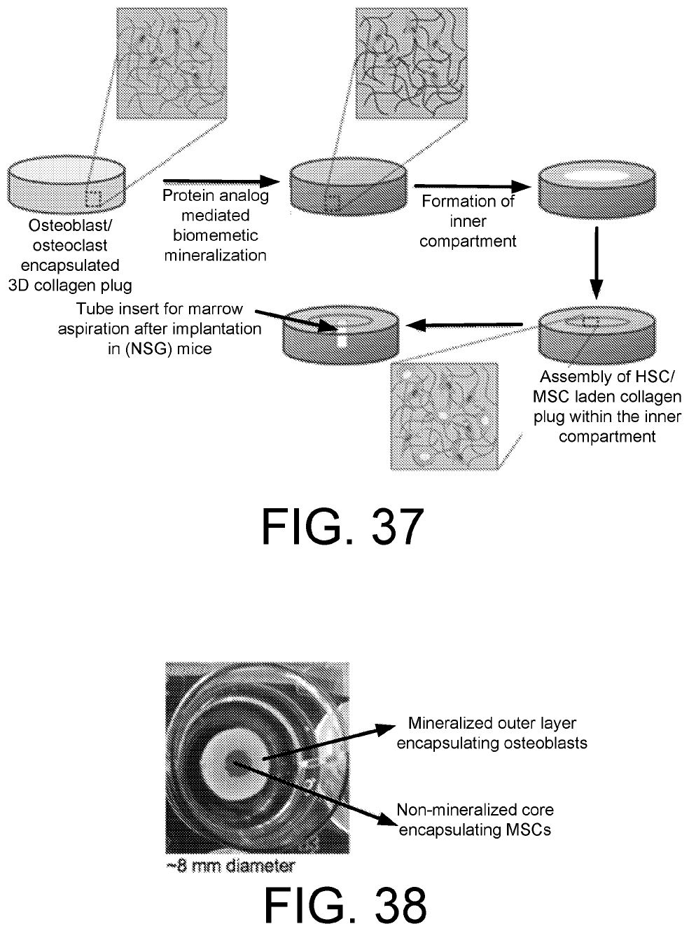

Ring Protocol: Mineralized cell seeded collagen rings can be created by removing an inner area of the endosteal niche disk using of a biopsy punch. Cell-seeded hydrogel ‘plugs’ are then added to the center void and allowed to cross link at 37° C. in a humid atmosphere containing 5% CO₂ for at least 30 minutes and up to 1 hour. Appropriate growth media is then added, depending upon experimental conditions. FIG. 37 presents a ring design of this embodiment, showing a mineralized outer layer encapsulating osteoblasts and a non-mineralized core encapsulating mesenchymal stem cells.

In some embodiments the cells are encapsulated in a collagen matrix and mineralized (as shown before), creating the endosteal niche. The endosteal niche can be created with different hydrogels, with and without cells. Later, a second layer of hydrogel containing stem cells may be introduced to create the vascular niche.

Disk Protocol: Disks are created by using applying approximately 100 μl osteoblast seeded collagen onto the center of a well within a 24-well tissue culture plate. Sizes may be scaled up or down by changing well size and volume used. This mixture is allowed to cross link at 37° C. in a humid atmosphere containing 5% CO₂ for at least 30 minutes and up to 1 hour. One milliliter of warmed mineralization media is added to the well and the plate is placed upon a 2-d shaker inside a 37° C. incubator with humid atmosphere and 5% CO₂. Media is removed daily and fresh media is added for three days after which DMEM containing, 1 g/L sucrose, 25 mM HEPES, 10% FBS, and 1% antibiotics is used. Shrinkage of the disks is normal and may be up to 50% of original size after mineralization.

Experiments are setup by removing the disk from the original well using forceps and placing into a smaller well. Care is taken to lie the disks as flat as possible to allow even coverage of the cell-laden hydrogels, e.g. Matrigel mixtures detailed herein or known in the art. These hydrogels are applied to the top of the disk and allowed to set. Appropriate media, as determined by experimental conditions, are then applied to the well. Media is replaced periodically or when pH change is noted if an indicator dye such as phenol red is present.

One embodiment herein includes a dual-layered composition including: a first layer including a hydrogel containing stem cells; and a second layer including a mineralized collagen containing osteoblasts; wherein the first layer and second layer contact each other.

In some embodiments, the two layers of the dual-layered composition are substantially the same in thickness. In some embodiments, the two layers of the dual-layered composition have substantially the same thickness and surface area.

In some embodiments, the dual-layered composition is disc-shaped, wherein each layer of the disc has approximately the same circumference and thickness, forming two substantially parallel surfaces for each layer, wherein one of the substantially parallel surfaces for each layer is in contact with a corresponding surface of the other layer. In other embodiments, each layer of the disc forms a cylinder having the same diameter and circumference, but different heights, i.e. the two layers have the same upper and lower surface dimensions, but differ in thickness.

Microvascular Capillaries:

Other embodiments of the designs and methods provided herein include pericyte-supported vascular endothelial capillaries, vascularizing the endosteal niche. Endothelial cells can be added before or after mineralization. In one example, collagen hydrogel or another hydrogel is pipetted onto 24-well plates and allowed to undergo fibrillogenesis in a humidified 5% CO₂ incubator at 37° C., for instance for 30 min. For pericyte-supported endothelial tubulogenesis, human umbilical vein endothelial cells (HUVECs) and human mesenchymal stem cells (hMSCs) may be co-encapsulated in the hydrogel at a ratio of 4:1 to a final concentration of 2.5×10⁶ cells/mL, cultured for 3 days, and allowed to form vascular capillaries. The cells self-assemble into a vascular capillaries in a process of vascular morphogenesis. The constructs may then optionally be subjected to the mineralization process described herein. Methods for generating microvascular capillaries have also been described, for instance, in Thrivikraman et al. (Nature Comm., 10(1):3250, 2019) and WO 2020/069033.

Microfluidics:

The bone marrow architecture can be implemented in many ways. In one embodiment, the biomimetic bone marrow can be implemented on a microfluidic chip (bone marrow on a chip). The right implementation of the system will allow to replicate the organ structural organization and physiological functions, as well as to have a high control on the organ microenvironment—e.g., oxygen and nutrient levels, drug exposure—and the chip output, providing valuable information for cancer biomarker discovery and precision medicine, among others. The proposed model can be implemented in 2D or 3D and contain or not specific cell types, which can be seeded with or pre-encapsulated. The microfluidic device can be made of any number of materials including but not limited to Acrylic, plastic, PDMS, polymers and any combination thereof. The niches may or may not be separated from the culture media channel via a permeable barrier.

The microfluidic device can be connected to other organ chips such as breast, liver, etc. to investigate a myriad of subjects including, but not limited to, metastasis, drug screening, drug toxicity, and organ system interactions.

Layer by Layer:

In one embodiment, the microfluidic device is a layer by layer design made of acrylic material—or similar—with a PDMS bottom (see FIGS. 3 and 4). The device includes a mineralized compartment encapsulating mature bone cells (endosteal niche) and an inner soft compartment incorporating the elements of the hematopoietic niche (vascular niche) (see Thrivikraman et al., Nature Comm., 10(1):3250, 2019; WO 2020/069033). The Endosteal Niche can be modeled by a mineralized hydrogel (for instance, collagen) encapsulating cells (for instance, osteoblasts and/or MSCs). The vascular niche is modeled by a hydrogel (for instance, MATRIGEL®) containing cells, for example, Mesenchymal Stem Cells (MSC) and Hematopoietic Stem Cells (HSC). The Endosteal Niche can be separated from the Culture Media (red) with a biocompatible medium permeable barrier. The permeable membrane may or may not include extracellular matrix (ECM) lined with Endothelial Cells (on one or both sides).

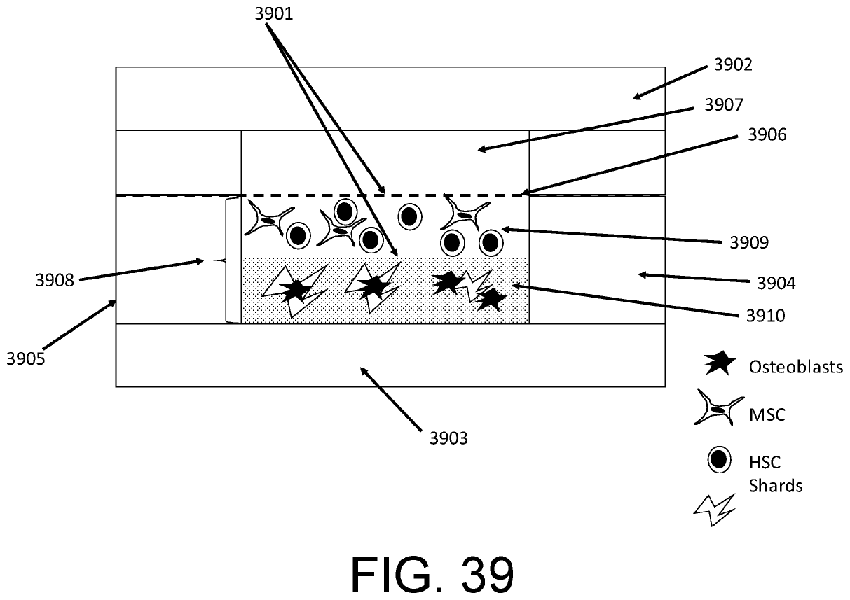

FIG. 39 illustrates an embodiment of the present invention, a layered design wherein the bone niche environments (3908) are maintained in a housing including a top (3902), a bottom (3903), and sides (3904 and 3905). Housings of this type may include any material(s) sufficient for maintenance of the biological activities within. A biocompatible permeable barrier (3906) separates channel for media (3907) from the niches below (3908). In the present example the vascular or hematopoietic niche (3909) is pictured containing mesenchymal stem cells (MSCs) and hematopoietic stem cells (HSCs) and the endosteal niche (3910) is pictured as containing osteoblasts. It will be understood that the order of the vascular or hematopoietic niche (3909) and the endosteal niche (3910) can be reversed.

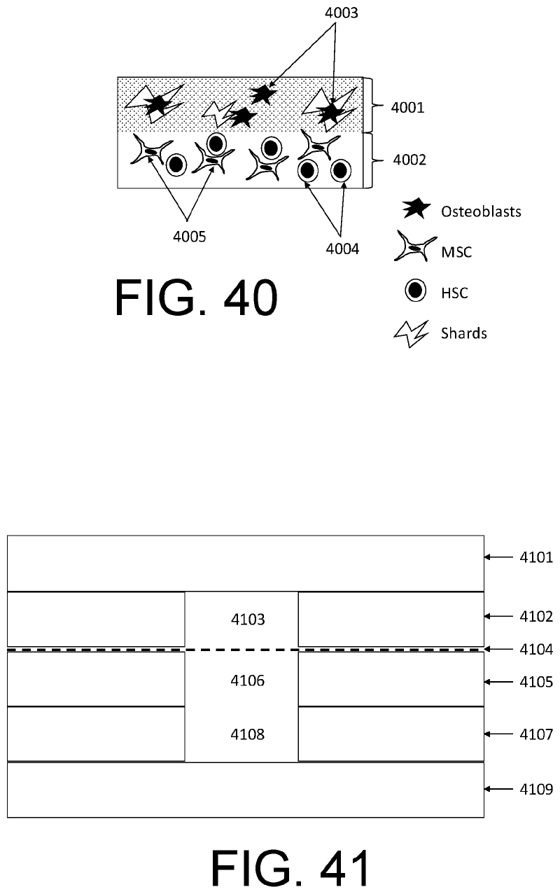

FIG. 40 provides an enlarged view of a two-layered system of this invention including a mineralized hydrogel layer (4001) serving as an endosteal niche and a non-mineralized layer acting as a vascular or hematopoietic niche (4002). The endosteal layer contains osteoblasts (4003) and the vascular layer contains hematopoietic stem cells (4004) and mesenchymal stem cells (4005).

FIG. 41 represents separated elements for an assembly useful as a microfluidics device. Between FIG. housing layers, an upper layer or cover (4101) and a base or bottom layer (4109). In some embodiments, the cover (4101) may include an acrylic and the bottom layer (4109) may include a PDMS or Acrylic material. The sheets including layers (4102), (4105), and (4107) may also include acrylic or PDMS. The permeable layer (4104) may include PDMS or polyethylene terephthalate (PETE) capable of allowing passage of desired media and contents. One commercially available membrane for this purpose is the PE1009030 10.0 micron, 90 mm polyester (PETE) membrane filters available from Sterlitech Corporation (Kent, Wash., USA). When connected, the upper channel (4102) will allow passage of media through the device, middle channel (4106) will include the non-mineralized vascular or hematopoietic niche, and the lower channel (4108) will include the endosteal niche.

In one embodiment, designs containing layers as those of FIG. 41 can have dimensions that allow a desired media to move via a microfluidics pump from one location along upper channel (4103) to another, with the media being accessible or in fluid contact or communication with the vascular (4106) and endosteal (4108) niche channels through the porous membrane (4104). In one embodiment, the upper channel (4103) is from about 0.5 mm wide to about 3 mm wide and from about 300 μm to about 600 μm thick or tall. The middle/vascular channel (4106) and the lower/endosteal channel (4108) may be, from about 0.5 mm wide to about 3 mm wide and from about 100 μm to about 300 μm thick or tall. Examples for the slides or panels including the top (4101) and bottom (4109) include commercially available 75 mm×25 mm or 95 mm×45 mm glass slides.

In another embodiment, the device is a sandwich device referred to as a SuperHERO™ chip in which the cell layer cross section is 950 microns deep×1 mm wide; the media layer is 400 microns deep×1.25 mm wide; the cell layer volume is 25 μL; and the media layer volume is 15 μL.

A pump system capable of moving microfluidic volumes of media can be connected to the design of FIG. 41 at one end of the upper channel (4103). Work for the present designs was accomplished using a LEGATO® 111 Syringe Pump (available from KD Scientific Inc., Holliston, Mass., USA) in combination with a 5-10 ml Luer Lock syringe. Media flow rates may vary depending upon the application indicated. In some embodiments, a flow rate of from about 0.5 μl/minute (0.03 ml/hr) to about 2.0 μl/minute (0.12 ml/hr). In some embodiments, a media flow rate of about 1 μl/minute (0.06 ml/hr) is indicated. In other embodiments, a media flow rate of about 1.5 μl/minute (0.09 ml/hr) is indicated.

A design as seen in FIG. 41 may be prepared as two separate assemblies that are then combined. The first assembly includes the lower plate (4109) and the endosteal plate (4107) and the second assembly includes plates (4101), (4102), and (4105) and permeable membrane (4104). In the first assembly, endosteal plate (4107) is adhered to the lower plate (4109) and lower channel (4108) is filled with the desired elements of the endosteal niche, such as osteoblasts on a three-dimensional collagen structure, which is then cross-linked and mineralized via the methods herein. Elements (4101) through (4106) are then layered together and the desired elements of the vascular niche, such as MSCs on collagen) are introduced into channel (4106). The first and second assemblies can then be joined, and a desired culture media may be run through the device via upper channel (4103).

In more detailed steps, this assembly may be accomplished as follows:

Lower assembly (4107-4109): Fabricate the different layers on the appropriate material (Acrylic or PDMS). Acrylic layers may be machined using laser cutting or computer numerical control (CNC) cutters. Attach lower plate (4107, green) to a cover glass or acrylic layer using Double Side Tape (DST). In case of using PDMS for the bottom substrate, a plasma treatment, including a treatment with (3-aminopropyl) trimethoxysilane (APTES), may be used to improve the bonding. Clean the lower assembly (4107-4109) using isopropyl alcohol (IPA). Sterilize with 70% Ethanol and UV for 30 min. Surface treatment of the first layer (4107-4109) is completed with: O₂ plasma (90 s), followed by Fibronectin (4° C. Overnight or Room Temperature for at least 2 hours). Wash the lower assembly with media. Prepare the Osteoblasts with the collagen. Keep in ice to prevent cross-linking. Fill the channel (4108) with the Endosteal niche (e.g., Osteoblasts on collagen) using a Pipette (60 μl). Cross-link the endosteal niche components in an incubator (45 min, 37° C.). Place the lower assembly (4107-4109) on a petri dish and culture with Osteoblasts Cell Medium (1 Day, 37° C.). Mineralize the endosteal niche in a Petri Dish (3 days, 37° C., Shaker)

Upper assembly (4101-4106): Assemble upper assembly layers using double side tape. Rinse with IPA. Sterilize the upper chip and the tubing with Ethanol and UV light. Attach the upper assembly and the bottom assembly using double side tape. Prepare the vascular niche (e.g., Stem Cells) with the collagen and store in ice until use to prevent cross-linking. Use the inlet for the middle channel (4106) to flow the vascular niche using a pipette. Cross-link the hydrogel in the incubator (45 min, 37° C.). Concurrently, wash intended pump tubing with Ultra-Pure Water using a syringe. Water bath and degas the culture media at 37° C. for at least 30 min. Connect the tubing to the upper media channel (4103). Run culture media through the upper (red) channel in the incubator (Flow rate: 1 μl/min=0.06 ml/h; Syringe: with Luer Lock 5-10 ml; Pump: LEGATO® 111 Syringe Pump).

Niche Separator:

Also provided is a device for preparing an endosteal niche and a vascular niche together.

An example of such a device includes: a body having a top and bottom; a first open channel having a first length and extending substantially straight from a first point in the device to a first exit, the first exit opening out of the body, wherein the first open channel extends through the body at or adjacent to the bottom of the body; a second open channel having a second length and extending substantially straight from a first point in the device to a second exit, wherein the second channel is parallel to the first open channel and the second exit opens out of the body parallel to and in open communication with the first exit; a first opening for receipt of material, wherein the first opening is in open communication with the first open channel; and a second opening for receipt of material, wherein the second opening is in open communication with the second open channel; wherein the elements above are in positions such that endosteal niche materials, as described herein, introduced into the first opening for receipt of material, enter and are directed along the first open channel and exit the body as an endosteal niche extrusion through the first exit; and vascular or hematopoietic niche materials, as described herein, introduced into the second opening for receipt of material, enter and are directed along the second open channel and exit the body through the second exit as a vascular or hematopoietic niche extrusion; and the vascular or hematopoietic niche extrusion exiting the second exit is deposited on the endosteal niche extrusion exiting the first opening.

In some embodiments, the body of the device is substantially flat on the bottom. In some embodiments, the top of the device is substantially flat. In other embodiments, the top and bottom of the device are both substantially flat, and the planes of their surfaces are substantially parallel to each other. In other embodiments the first open channel and the second open channel are in open communication to each other as they pass through the body.

In one embodiment, the microfluidic device, using a phase separation mechanism, is made of printed resin or with PDMS through micro molding and layer assembly. Similar to the previous microfluidic device design, a mineralized compartment encapsulating mature bone cells (endosteal niche) and an inner soft compartment incorporating the elements of the hematopoietic niche (vascular niche). The Endosteal Niche can be modeled by a mineralized hydrogel (for instance, collagen) encapsulating cells (for instance, osteoblasts and/or MSCs). The vascular niche is modeled by a hydrogel (such as MATRIGEL®) containing cells, for example, Mesenchymal Stem Cells (MSC) and Hematopoietic Stem Cells (HSC).

The Endosteal Niche is initially added to the device as a liquid hydrogel via a dedicated inlet confined within the specific channel by a niche separator. After hydrogel solidification, the endosteal niche can be cultured to achieve proper levels of mineralization before the vascular niche is added. These two layers form an interface that is not interrupted by other materials. Endothelial cells can be seeded to the exposed vascular niche using the remaining culture media channel for the creation of an endothelial monolayer. Culture Media can lastly be perfused through any open channel. In this configuration, the top channel of the system is used so that nutrients will initially diffuse to the past the monolayer, into the vascular niche and finally into the endosteal niche.

It is also contemplated that microfluidic device embodiments may employ channels with patterned small ridges, such as PhaseGuides™ capillary pressure barriers (Mimetas, The Netherlands).

Niche Separator:

Resin niche separators are designed, for instance using CAD for both positive and negative versions. Positive resin niche separators are printed and cleaned with ethanol, following manufacturer recommendations. The printed samples undergo further post processing steps of curing under UV lights for 20 minutes (Triad II, Dentsply) and overnight storage at 80° C. to reduce the amount of unreacted monomer present. These prepared parts are used as impression templates in which Polydimethylsiloxane (Sylgard 184, Dow Corning) is cast around the template. After solidification, the printed resin part is removed from impression material. Fluidic connection ports are punched (e.g., using a biopsy punch) into the device, and fluidic connectors can be attached during device use.

Negative niche separators are 3D printed using the same resin as the negative forms. These versions are designed to be the final form of the micro fluidic device, not requiring additional molding steps. The cleaning process for these resin devices can be the same as the positive devices, and follows the previously described cleaning and post processing steps. PDMS molds are attached to glass coverslips via plasma treatment (Plasma Cleaner PDC-32G, Harrick Plasma). Resin devices were attached to glass using super glue adhesive.

HERO™ Sandwich Chip. The Hero™ chips are composed of two channels that may or may not be separated by a biocompatible permeable barrier. HERO™ sandwich chips can be made of different materials, including for instance PDMS or acrylic.

PDMS Hero™ chips are composed of three parts (from top to bottom): Upper channel: PDMS, for instance at least 3 mm thick with a 0.5 mm thick channel; porous membrane (for instance, a PETE membrane with a porosity of 10 μm); and Bottom channel: PDMS, for instance of about 1 mm thick with 0.4 mm thick channel. A method of producing a representative PDMS HERO chip is described in Example 8.

The PDMS molds with the desired channel shape and dimensions are fabricated using standard techniques (such as CNC, laser cutting, and so forth). Prepared PDMS (ratio 1:10) is pour on the aforementioned molds, then degassed and cured overnight at room temperature. Alternatively, this can be cured at a higher temperature for a shorter period (for instance, 80° C. for 2 hours). Inlet and outlet holes are added, using a puncher.

The PDMS chips are fused with/attached to a PET membrane using standard procedures. For instance, PDMS is rinse with alcohol (such as isopropyl alcohol; IPA), dried with nitrogen, then immersed in IPA under sonicate for 20 minutes. The treated PDMS is then tried with nitrogen again, and placed at 80° C. for at least 20 min. In the meantime, PET membrane is placed in the appropriate support, then cleaned/etched using oxygen plasma (e.g., O₂ plasma (35 W, 45 s)). The PET membrane is then moved to a new support, and a thin layer generated over the membrane by spreading an APTES dilution (5% in deionized water) over it and placing at 70° C. for 30 min.

The PDMS and membrane is then placed on a new support, and oxygen plasma etched again (35 W, 15 s), as is another PDMS chip (O₂ plasma (35 W, 45 s)). The surfaces are brought together and subject to mechanical pressure for an hour. This process is repeated for subsequent layer(s).

The assembled chip is rinsed with IPA, then sterilized with 70% ethanol followed by UV treatment for 30 minutes. The sterile assembled sandwich chip is treated overnight with

A representative Acrylic Hero™ chips includes five layers (from top to bottom): Upper cover made of Acrylic, Culture Media channel layer made of Acrylic, porous membrane (such as a PETE membrane with porosity of 10 μm), hydrogel layer made of Acrylic, and bottom cover, for instance made of glass or high precision acrylic.

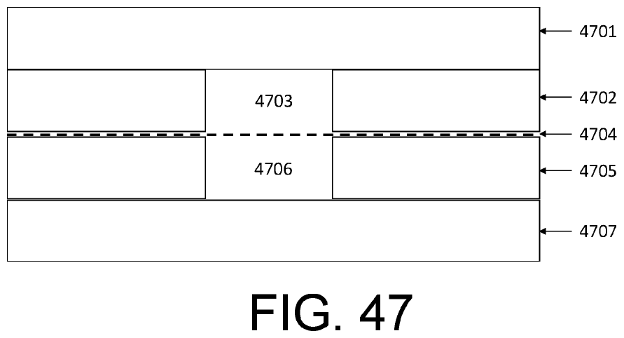

In FIG. 47, an example of an acrylic hero includes an acrylic cover (4701), a culture media channel layer, represented by layer material or sheet 4702 and channel 4703, a permeable barrier or membrane (4704), a vascular and/or endosteal channel layer, represented by layer material or sheet 4705 and channel 4706, and a glass slide 4707.

A method of producing a representative Acrylic HERO chip is described in Example 9.

Dual Media Model:

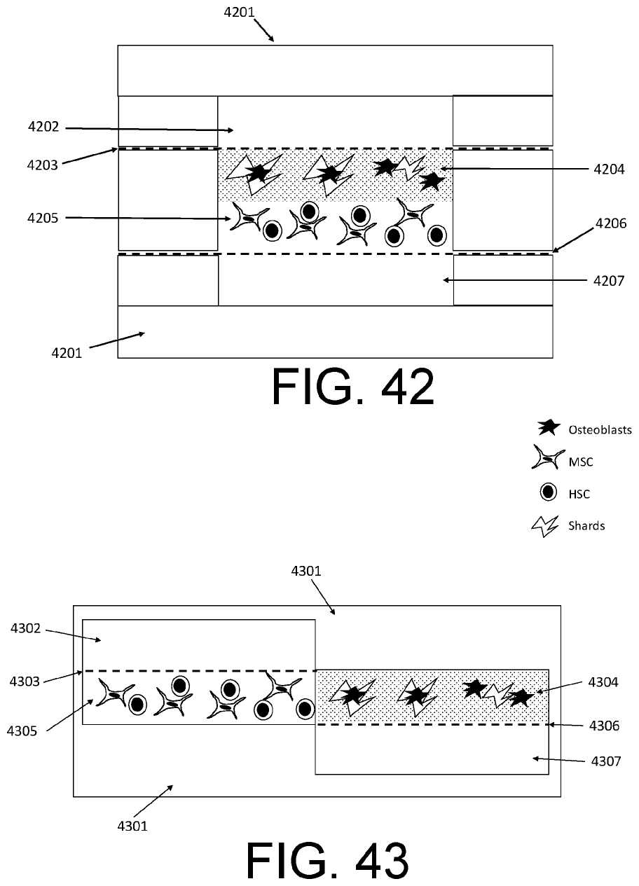

In one embodiment, the device is composed of four different regions including two medium/media layers, and two niche regions (endosteal niche and stem cell/vascular niche); see three-dimensional FIGS. 42, 43, and 44. FIG. 42 depicts such a device in which the layers are maintained in a housing (4201). Media layer A (4202) is adjacent to the endosteal niche (4204) with a permeable barrier (4203) in between. On the other side of the device, Medium layer B (4207) is adjacent to Stem cell/vascular niche (4205) with a permeable barrier (4206) in between.

A naturally-formed physical contact interface (not shown) may be created between the Endosteal niche and the stem cell niche; an interface stabilizer design is used to separate these two niches. To form this separation of two niches, a premixed hydrogel at liquid phase (endosteal niche) is infused into the device through an inlet of Medium A. The premixed hydrogel will fill only one side of the interface stabilizer due to the surface tension at the hydrogel interface. The first premixed hydrogel at liquid phase will crosslink and form a non-liquid, more solid layer after providing suitable crosslinking conditions. This process results in a partially channel of hydrogel, which is adjacent to the Medium A layer, while the other half of the channel (adjacent to the lower Medium B layer) is empty (contains only air phase). The first infused hydrogel will be mineralized by the aforementioned method, and it will form the endosteal niche. The second premixed hydrogel (stem cell niche) is infused through the corresponding inlet after the mineralization of the endosteal niche is completed. The second premixed hydrogel will fill the area between the endosteal niche and the permeable barrier by simple fluidic dynamics. This filling process will create good physical contact the between endosteal niche and the stem cell niche. The second premixed hydrogel will crosslink and form a solid layer to create the stem cell niche. An embodiment with two separated medium layers and two contacting niches is formed. The individual medium layers (A and B) will provide nutrient and oxygen to the endosteal and the stem cell niche from opposite sides, respectively. The design with two separated medium layers gives flexibility of infusing different types of medium that are suitable for each niche, and provides the flexibility of introducing different perturbations corresponding to experimental and/or testing purposes. The perturbation can be different levels of oxygen, supplements, drugs, and cell suspension medium, among others. By changing the perturbation and corresponding flow ratio or flow rate, these perturbations can be restricted to one niche or a gradient within the two niches can be created and controlled. Two different perturbations (e.g., drug) can be introduced to influence each niche or can be controlled to give more influence on one niche and less on the other one. An oxygen gradient can be produced to resemble the healthy human physiological condition or to resemble human physiological conditions with progression of specific diseases, such as cancer. Two separated medium layers also provide the flexibility to sample the chemokine, cytokine or other secretions from each niche, and separated biopsy is applicable.

FIG. 43 provides a three-dimensional view of another embodiment of a device herein wherein, within body (4301), the endosteal niche (4304) is found side-by-side and in contact with the vascular niche (4305). A first media channel (4302) is substantially associated through permeable membrane or barrier (4303) with the vascular niche (4305). A second media channel (4307) is substantially associated through permeable membrane or barrier (4306) with the endosteal niche (4304). Such a design may be useful for testing an agent's effect on the vascular niche in coordination with another agent's effect on the endosteal niche.

In different embodiments or testing methods, it is understood that the two fluid channels may provide the same media or different mediums to the endosteal and hematopoietic niches involved. In some embodiments the fluid mediums are different to establish a concentration gradient through the bone marrow organ model.

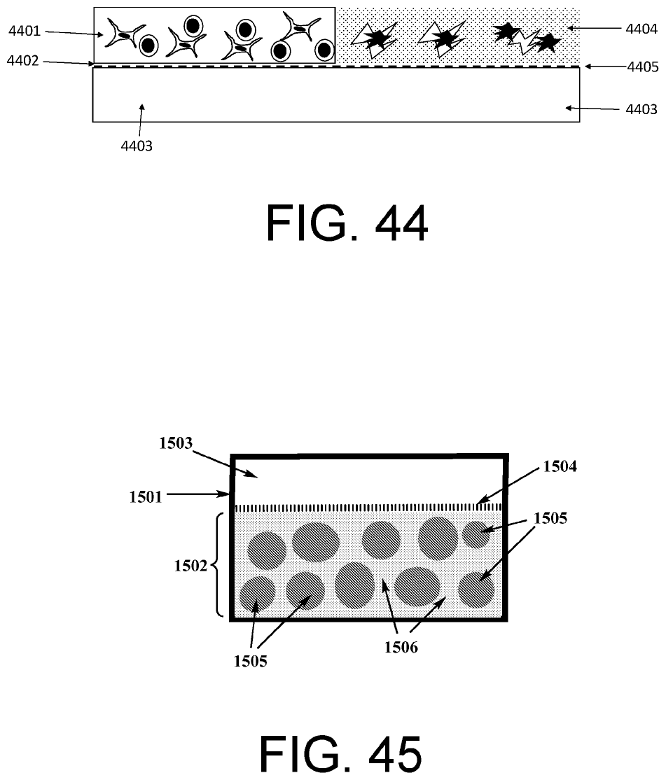

FIG. 44 provides another embodiment without a housing shown, which may or may not be present, as desired. In this design, the vascular niche (4401) is adjacent endosteal niche (4404), and both are separated from the same media channel (4403) by corresponding permeable barriers (4402) and (4405), respectively. This design may be useful for testing the same agent for its activity regarding both niches.

FIG. 45 provides a lateral cross-sectional view of another embodiment wherein a number of endosteal niche segments (4505) in the form of spheres or spheroids are interspersed within the vascular niche materials/matrix (4506) in the same compartment (4502). Individual hydrogel/collagen spheres may be created separately before being combined in a single chamber (4502) separated by a permeable barrier (4504) from a media channel (4503). The embodiment in FIG. 15 contains each element in housing (4501).

In Vivo Model:

In one embodiment, a biomimetic bone marrow is implemented in vitro via different geometries and configurations including but not limited to ring, disk, or sandwich models. This can be implanted in vivo (human, mice, etc.) or be cultured in vitro. These structures can later be implanted in vivo or kept in vitro and cultured. This model offers in vivo complexity that can be used to investigate a myriad of events including but not limited to engraftment, migration, tissue engineering and tissue development. This approach is an improvement over current models that only mimic the endosteal niche, or take months to create a bone marrow niche from primary MSCs. By creating some of the components of the marrow niche separately in vitro (endosteal, endothelial, etc.) we can accelerate creation of an engineered bone marrow niche in vivo. This also allows for manipulation of different components of the niche. For example, primary MSCs from individuals with a hematologic malignancy, such as acute myeloid leukemia, can be used to create an individualized niche that supports leukemia cells in a xenograft model.

Ring: In another embodiment the cells are encapsulated in a collagen matrix and mineralized (creating the endosteal niche), a hole is created via biopsy puncher and is filled with a second layer of hydrogels containing stem cells is introduced creating the vascular niche (ring model; see FIG. 37).

Disk: In one embodiment, the cells are encapsulated in a collagen matrix and mineralized, creating the endosteal niche, then a second layer of hydrogel containing stem cells is introduced creating the vascular niche (disk model). This can be formatted, for instance, as a set of stacked discs representing the vascular layer or niche and an endosteal layer or niche. In another embodiment, shards are embedded in the vascular niche and the structure is implanted in mice or another test animal.

Disease Models:

The implementations described herein can be used with primary cell samples to create disease-specific models that can then be used to screen drugs, small molecules, radiation, antibodies, etc.

Primary marrow stromal cells can be cultured and expanded in vitro (Traer et al., Cancer Res 76(22):6471-6482, 2016) from a number of different hematologic malignancies and disorders, including acute leukemias, chronic leukemias, marrow failure syndromes, lymphomas, myelomas, and solid tumor metastasis to the bone marrow. This retains the unique expression of the diseased state microenvironment they were derived from (Viola et al., Br J Haematol., 172:978-992, 2016). The disease-specific MSCs can then be combined with malignant cells, for example leukemia cells, and/or normal HSCs, to recreate the native features of the diseased bone marrow microenvironment. This approach can be used to create a personalized model of the bone marrow with primary diseased mesenchymal cells and diseased hematopoietic cells (leukemia cells for example) derived from the same patient. The addition of diseased primary endothelial cells is another component that may be added to the model. The important interaction between bone marrow stromal cells and malignant hematopoietic cells is recreated with this approach.

Primary cells can be used to create personalized disease-specific models for a wide variety of both hematologic malignancies (leukemias, lymphomas, myelomas, etc.) and pre-malignant conditions, such as clonal hematopoiesis of indeterminant potential (CHIP), clonal cytopenias of undetermined significance (CCUS), monoclonal gammopathy of undetermined significance (MGUS), and monoclonal B-cell lymphocytosis among others.

Primary MSCs and HSCs also can be used to create a bone marrow microenvironment to study metastatic models of disease. Solid tumors such as lung cancer, prostate cancer, breast cancer, thyroid cancer, etc. can develop metastases to bone marrow. A model of the bone marrow can be made with either MSCs and HSCs from normal cells, or by using primary MSCs and primary HSCs derived from patients. Primary endothelial cells may or may not be included as well. Metastatic cells using either cell lines or from primary patient samples can be added to the model directly to recreate bone marrow metastasis. This approach allows a model to study cancer cell sensitivity within the unique microenvironment of the metastatic site.

Disease models can then be used to screen drugs and pre-clinical small molecules (or combinations), for instance using a delivery system that mimics normal peaks and troughs of drug concentration. This is achieved with the pump delivery system of the vascular channel. Using the pump better recapitulates the pharmacokinetics of drug delivery in vivo. The longer survival of primary cells in the marrow on a chip model also allows screening of drugs that do not have immediate cytotoxic effects on disease cells. One example of this is drugs that lead to differentiation of leukemia cells, such as hypomethylating agents, tretinoin, and IDH inhibitors. These drugs cannot be assayed with normal cell culture models, because the cells do not survive long enough to measure differentiation. However, in chip models described herein the cells survive long enough to evaluate these changes. In addition, the effects of drugs on the normal HSCs can also be evaluated with the herein described model. For example, disease cells from leukemia patients may contain some normal HSCs (or normal HSCs can be added) that are repressed by the diseased leukemia and stromal cells. If the diseased cells are successfully treated using any of the therapies described above, the normal HSCs and MSCs may be able to expand and restore normal hematopoiesis. This can be used to evaluate the therapeutic window of a drug, or approaches to specifically reduce toxicity to normal HSCs. Another possibility is evaluating the effects of drugs and small molecules that target diseased stromal cells, and the subsequent effect on normal and diseased hematopoietic cells in the microenvironment either alone or in combination with other drugs (Javidi-Sharifi et al., Elife. 2019 Mar. 29; 8. pii: e47174).

Other types of therapy that can be evaluated with the described bone marrow models, include radiation, antibodies (or antibody fragments), antibody-drug conjugates, liposomal-enclosed drugs, and engineered cellular therapies such as chimeric antigen receptor T cells (CAR T). The chip model creates a more native marrow microenvironment to model how these molecules enter from the vasculature, enter the marrow space, and affect diseased cells in the presence of the surrounding cells of the microenvironment.

In addition to evaluating the direct effects of drugs on diseased cells, the model can also be used to evaluate how diseased cells affect normal cells. For example, diseased hematopoietic cells, such as leukemia cells, can be added to normal MSCs to study how the leukemia cells alter the expression and growth of the normal MSCs to support their own growth. Conversely, stromal cells from diseased states can be combined with normal HSCs to evaluate the effects on normal hematopoiesis. Alternatively, normal HSCs and MSCs can be used to make a model of the bone marrow and then exposed to secreted proteins or exosomes derived from diseased cells (leukemia cells, lymphoma cells, metastatic solid tumor cells, etc.) or diseased stroma (Javidi-Sharifi et al., Elife. 2019 Mar. 29; 8. pii: e47174) to determine the effect on hematopoiesis and/or stromal cells in the marrow microenvironment. This will allow studies of disease progression, reprogramming of the marrow microenvironment (hematopoietic and stromal cells), and pre-metastatic remodeling; and the potential to test and analyze drugs, antibodies or proteins that might modulate these effects.

Normal HSCs and MSCs can also be used to create a normal bone marrow model that is exposed to drugs or small molecules used to treat other medical conditions or malignancies, or toxins that affect the marrow, to explore the marrow-specific side effects on stromal, hematopoietic, and endothelial cells. This can be used to test additional drugs or proteins designed to specifically protect the marrow cells from exogenous insults.

Performing a Bone Marrow “Biopsy”

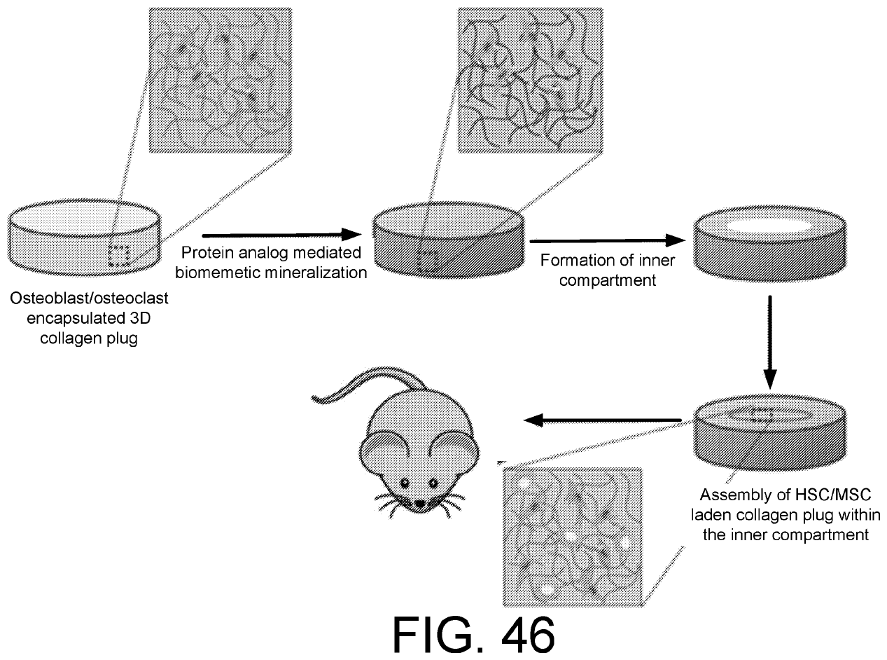

Incorporation of a Plug on the in vivo/in vitro model: Different elements can be incorporated in the design to facilitate access to content of the engineered bone marrow model in a “biopsy like” manner. In one embodiment, a plug can be incorporated in the design to facilitate marrow aspiration, as indicated in FIG. 46. Biopsies may also be conducted on a bone marrow chip or bone marrow model/device as described herein.

In order to have access to the chip content and carry out further analysis, a biopsy-like procedure can be operated directly on the microfluidic chip without disturbing chip function. A puncher can be used to get a biopsy of the chip channel including the region of interest. A dummy tubing can be left behind in order to avoid leakage and preserve the structure and operation of the chip.

Methods of Analysis with SEM, TEM, uCT

Characterization of the microdevice content is typically done via light-microscopy. However, the bone matrix has myriad characteristics that may benefit from nanoscale analyses, such as collagen mineralization and cell-matrix interactions.

For that, electron microscopy (EM) is recommended. In one embodiment, the device is fabricated to be compatible with conventional EM fixation and embedding protocols while tissue is kept on-chip.

For instance, such a device is configured to enable sectioning of fixed material in cross-section or longitudinal section, and to enable imaging in conventional electron microscopes such as SEM, TEM, as well as in tomography-based equipment, such as μCT.

Methods of Analysis with Cyclic Immunofluorescence (CycIF)

Cyclic Immunofluorescence (CycIF) is a highly multiplexed immunofluorescence imaging where samples are repeatedly stained and imaged using a conventional fluorescence microscope. It uses simple reagents and existing antibodies to construct images followed by fluorophore inactivation, in a cyclic manner.

This method can be used for any of the herein described implementations of engineered bone marrow. For instance, bone marrow cell(s) on a microfluidic device can be labeled with different antibodies through the media or cell compartment, followed by optional nuclei stain, and imaging, followed by antibody deactivation with quenching agents, and then re-staining again in a cyclic manner. Typically, this is used to identify particular stromal cells, diseased cells, and/or normal cells with the marrow compartment, including their proximity to each other. An advantage of CycIF is that the cells remain in the same position during multiple rounds of staining and the images can be merged with software for analysis.

Matrix Mineralization Methods

Methods for preparing a mineralized matrix for the endosteal niche for use in the designs and combinations herein may be accomplished in specified methods.

A mineralized matrix of polymer chains and living cells, may be prepared by a method including: combining the living cells with a fluid medium and unlinked polymer chains to create a first composition; treating the first composition with a cross-linking agent or cross-linking treatment sufficient to cross-link the polymer chains to form a cross-linked polymer matrix including the living cells; and treating the cross-linked polymer matrix including the living cells with at least one mineralizing solution for a time sufficient to form the composition of a mineralized cross-linked polymer matrix including the living cells.

Also useful is a method of preparing a composition of a mineralized matrix of polymer chains and living cells, the method including: combining unlinked polymer chains with a fluid medium to create a first composition; treating the first composition with an agent or treatment sufficient to cross-link or entangle the polymer chains to form a polymer matrix; combining the living cells with the cross-linked polymer matrix to form a polymer matrix including the living cells; and treating the polymer matrix including the living cells with at least one mineralizing solution for a time sufficient to form the composition of a mineralized polymer matrix including the living cells.

Another method of preparing a composition of mineralized Type 1 collagen matrix including living cells includes: combining the living cells with a fluid medium and acid solubilized Type 1 collagen chains to create a first composition; maintaining the first composition under conditions sufficient to cause the Type 1 collagen chains of the first composition to undergo fibrillogenesis and form a second composition including the living cells; and treating the second composition including the living cells with at least one mineralizing solution for a time sufficient to form the composition of a mineralized Type 1 collagen matrix including the living cells.

Another method of preparing a composition of mineralized Type 1 collagen matrix including living cells includes: combining a fluid medium and acid solubilized Type 1 collagen chains to create a first composition; maintaining the first composition under conditions sufficient to cause the Type 1 collagen chains of the first composition to undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.

Another method of preparing a composition of mineralized Type 1 collagen matrix including living cells includes: combining the living cells with a fluid medium and acid solubilized Type 1 collagen chains to create a first composition; adjusting the pH of the first composition to from 6-8, or from 6.5-8, or from 6.8-7.8, or from 7.0-7.8, or from 7.2-7.8, or from 7.2-7.6, or to about 7.6, and maintaining the first composition and maintaining the first composition at a pH from 6-8, or from 6.5-8, or from 6.8-7.8, or from 7.0-7.8, or from 7.2-7.8, or from 7.2-7.6, or to about 7.6, until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.

Another method of preparing a composition of mineralized Type 1 collagen matrix including living cells includes: combining a fluid medium and acid solubilized Type 1 collagen chains to create a first composition; adjusting the pH of the first composition to from 6-8, or from 6.5-8, or from 6.8-7.8, or from 7.0-7.8, or from 7.2-7.8, or from 7.2-7.6, or to about 7.6, and maintaining the first composition and maintaining the first composition at a pH of from 6-8, or from 6.5-8, or from 6.8-7.8, or from 7.0-7.8, or from 7.2-7.8, or from 7.2-7.6, or to about 7.6, until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.

Another method for preparing a composition of mineralized Type 1 collagen matrix including living cells includes: combining the living cells with a fluid medium and acid solubilized Type 1 collagen chains to create a first composition; adjusting the pH of the first composition to from 6-8, or from 6.5-8, or from 6.8-7.8, or from 7.0-7.8, or from 7.2-7.8, or from 7.2-7.6, or to about 7.6, and maintaining the first composition and maintaining the first composition at a pH of from 6-8, or from 6.5-8, or from 6.8-7.8, or from 7.0-7.8, or from 7.2-7.8, or from 7.2-7.6, or to about 7.6, and a temperature of from about 34° C. to about 40° C. until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.

Another useful method of preparing a composition of mineralized Type 1 collagen matrix including living cells includes: combining a fluid medium and acid solubilized Type 1 collagen chains to create a first composition; adjusting the pH of the first composition to from 6-8, or from 6.5-8, or from 6.8-7.8, or from 7.0-7.8, or from 7.2-7.8, or from 7.2-7.6, or to about 7.6, and maintaining the first composition and maintaining the first composition at a pH of from 6-8, or from 6.5-8, or from 6.8-7.8, or from 7.0-7.8, or from 7.2-7.8, or from 7.2-7.6, or to about 7.6, and a temperature of from about 34° C. to about 40° C. until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.

For each of the methods of preparation for a mineralized matrix, there is a further embodiment in which the same methods are used, and the mineralizing solution includes from about 3.0 mM to about 6.0 mM of calcium ions. For each of the methods of preparation, there is another embodiment in which the same methods are used, and the mineralizing solution includes from about 4.0 mM to about 5.0 mM of calcium ions.

For each of the methods of preparation, there is a further embodiment in which the same methods are used, and the mineralizing solution includes from about 1.5 mM to about 3.0 mM of phosphate ions. For each of the methods of preparation herein, there is a further embodiment in which the same methods are used, and the mineralizing solution includes from about 1.8 mM to about 2.5 mM of phosphate ions.

For each of the methods of preparation, there is a further embodiment in which the same methods are used, and the mineralizing solution includes from about 3.0 mM to about 6.0 mM of calcium ions and from about 1.5 mM to about 3.0 mM of phosphate ions.

For each of the methods of preparation, there is a further embodiment in which the same methods are used, and the mineralizing solution includes from about 4.0 mM to about 10.0 mM of calcium ions and from about 1.8 mM to about 20 mM of phosphate ions. See, for instance, Thrivikraman et al., Nature Comm., 10(1):3250, 2019; WO 2020/069033 for additional discussion on varying calcium and phosphate levels to influence mineralization.

It is understood that a “fluid medium” referred to herein indicates a biologically acceptable medium or growth medium that facilitates maintenance of the living cells in a given matrix. In some embodiments, the fluid medium is an aqueous medium including nutrients needed for cell growth and reproduction. Fluid media may also contain additional agents, not limited to antibiotic agents, antifungal agents, antiviral agents, buffers, anticoagulants, vitamins, salts, minerals, amino acids, nucleic acids, ribonucleic acids, fatty acids, lipids, O₂ and/or CO₂ gases, carbohydrates, serum proteins, cofactors, growth factors, cytokines, enzymes, hormones, signaling substances, antibodies, among others, or combinations thereof.

In other embodiments, the Type 1 collagen matrix in the methods and compositions herein is prepared by reconstituting acid solubilized Type 1 collagen. In further embodiments, the Type 1 collagen matrix is prepared by reconstituting acid solubilized Type 1 collagen to a final concentration of from about 0.5 mg/mL to about 5.0 mg/mL.

In some embodiments, the final matrix in the methods and compositions herein includes from about 1× to about 20× phosphate buffered saline (PBS). In other embodiments, the final matrix includes Dulbecco's Modified Eagle Medium (DMEM). In additional embodiments, the final matrix includes from about 5× to about 20×PBS and Dulbecco's Modified Eagle Medium (DMEM).

In other embodiments the living cells are present in the final matrix in the methods and compositions herein at a concentration of from about 1×10⁵ cells/mL to about 10×10⁵ cells/mL.

In some embodiments of the methods herein, the fluid medium in step a) of the methods of preparation herein is maintained at a pH of from 6-8, or from 6.5-8, or from 6.8-7.8, or from 7.0-7.8, or from 7.2-7.8, or from 7.2-7.6, or to about 7.6.

In embodiments of the methods herein, the matrix is maintained at a pH of from about 7.2 to about 8.0 during the mineralization process. In further embodiments, the matrix is maintained at a temperature of from about 34° C. to about 40° C. during the mineralization process. In still further embodiments, the matrix is maintained at a pH of from 6-8, or from 6.5-8, or from 6.8-7.8, or from 7.0-7.8, or from 7.2-7.8, or from 7.2-7.6, or to about 7.6, and at a temperature of from about 34° C. to about 40° C. during the mineralization process.

In some embodiments, the methods herein further include the steps above wherein the fluid medium in step a) includes a physiologically acceptable buffer or buffer system. In the systems and methods herein, the physiologically acceptable buffer or buffer system is understood to include any that are compatible with maintenance and/or growth and/or cellular activity of the cell type(s) included in the compositions and matrices herein.

In some embodiments, the buffer or buffer system includes a physiologically active amount of buffering agents alone or in a biologically acceptable fluid medium selected from the group of 4-(2-hydroxyethyl)-piperazineethane sulfonic acid (HEPES), Modified Dulbecco's Medium (DMEM), phosphate buffered saline (PBS or DPBS), sodium bicarbonate buffers, Roswell Park Memorial Institute (RPMI) or RPMI1640, Eagle's essential medium (EEM), Edinburgh Minimal medium (EMM), Hanks' salts medium (HMEM), Hank's Balanced Salt Solution (HBSS), Earle's Balanced Salt Solution (EBSS), Iscove's modified Dulbecco's Medium (IMDM), Osteoblast Medium (ObM), and fetal bovine serum (FBS), or combinations thereof.

In other embodiments the methods herein further include the presence of an inhibitor of hydroxyapatite formation, nucleation, or precipitation in the at least one mineralizing solution during the mineralizing process. In some embodiments, the inhibitor of hydroxyapatite formation includes a protein capable of inhibiting hydroxyapatite nucleation and precipitation in solution. In further embodiments, the protein capable of inhibiting hydroxyapatite is selected from the group of Osteopontin (OPN), Osteocalcin (OC), Osteonectin (ON), bone sialoprotein (BSP), dentine phosphoryn (DPP), dentin matrix protein 1 (DMP1), dentin sialophosphoprotein (DSPP), matrix extracellular phosphoglycoprotein (MEPE), chondrocalcin (CC), proline-rich proteins such as Proline-rich protein 1 (PRP1), Proline-rich protein 2 (PRP2), and Proline-rich protein 3 (PRP3), PRP1-T1, PRP3-T1, Histatin 5, MG1, MG2, Asialo_MG2, Amylase, statherin, cystatin S, cystatin SN, Cystatin S1, fetuin, and HSA. In some embodiments, the inhibitor of hydroxyapatite formation is Osteopontin, for instance 100 μg/mL Osteopontin.

The “mineralizing solution” or “mineralizing solutions” used herein refer to solutions, preferably aqueous solutions that provide an ionic source for a desired mineralization of a specified matrix herein. In some embodiments, the mineralizing solution includes a calcium ion solution. In other embodiments, the mineralizing solution includes a phosphate ion solution. In further embodiments, the mineralizing solution may include ions of magnesium, sodium, potassium, carbonate, iron, barium, boron, strontium, copper, and/or zinc.

In some embodiments, the mineralizing solution is one containing one or more sources of ionic minerals selected from the group of calcium phosphate, calcium carbonate, hydroxyapatite, strontium carbonate, barium carbonate, and calcium sulfate, strontium sulfate, calcium oxalate, magnesium-bearing calcium carbonate, and magnesium-bearing calcium phosphate.

In some embodiments, the mineralizing solution is a calcifying solution. In some embodiments, the calcifying solution includes calcium and phosphate ions. In some embodiments the calcifying solution includes a calcium salt selected from the group of calcium phosphate, calcium carbonate, calcium chloride (including those selected from the group of anhydrous CaCl₂, CaCl₂.H₂O, CaCl₂.2H₂O, and CaCl₂.6H₂O), calcium citrate, calcium glubionate, calcium gluconate, calcium acetate, and calcium lactate.

In other embodiments, the mineralizing solution is prepared using hydroxyapatite, octacalcium phosphate, tricalcium phosphate, carbonated hydroxyapatite, fluorinated hydroxyapatite, brushite, magnesium-containing hydroxyapatite, dicalcium phosphate dihydrate, and amorphous calcium phosphate.

Kits

Also provided are kits useful for examining, characterizing, working with, or using in experiments a bone marrow model as described herein. An example of the kit includes one or more bone marrow analysis device (such as a chip) as described herein; along with one or more solution(s) for use in or with the analysis device; and/or a liquid growth medium for culturing one or more cell types for use in the bone marrow model described herein; and/or a container or chamber in which the analysis device can be stored, operated, cultured, and or used. One specific kit example includes: (1) at least one bone marrow device; (2) an activation/chip coating solution(s) e.g. fibronectin or the like; (3) a matrix or components thereof, such as fibrinogen, thrombin, aprotinin etc. for use with a fibrin gel, or MATRIGEL®, or another hydrogel composition as described herein; (4) media and/or growth supplements; (5) devices and/or materials useful for loading cells into the device (for instance, when loading HUVECs on Emulate chips, a device is used to invert these levelly); and (6) instructions. Another specific kit embodiment includes at least one bone marrow model device; solution(s) to treat/coat the device prior to use; a hydrogel prepolymer; and cell culture media for cells for use in the device. Any of the provided kits may optionally also provide one or more types of reference cells, such as endothelial cells, MSCs and/or HSCs.

More generally, kits can comprise hydrogel matrix, reference standard molecules, solid supports, and medical devices described. Kits can include instructions, for example written instructions, on how to use the material(s) therein. Material(s) can be, for example, any substance, composition, polynucleotide, solution, etc., herein or in any patent, patent application publication, reference, or article that is incorporated by reference.

A kit can include a device as described herein, and optionally additional components such as buffers, reagents, and instructions for carrying out the methods described herein. The choice of buffers and reagents will depend on the particular application, e.g., setting of the assay (point-of-care, research, clinical), analyte(s) or cell(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about production of the device, physical properties of the device, date of expiration, batch or production site information, and so forth.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### First Set of Representative Embodiments

1. A bone marrow model including: a first microenvironment including a hydrogel with or without cells; and a second microenvironment including: a hydrogel with cells found in a normal hematopoietic niche of normal bone marrow; or a hydrogel with cells found in a condition selected from the group consisting of hematologic malignancies, marrow failure syndromes, pre-malignant clonal hematopoiesis, and solid tumor metastases to bone marrow.
2. A bone marrow model including: a first microenvironment including a mineralized hydrogel with or without cells; and a second microenvironment including: a hydrogel with cells found in a normal hematopoietic niche of normal bone marrow; or a hydrogel with cells found in a condition selected from the group of hematologic malignancies, marrow failure syndromes, pre-malignant clonal hematopoiesis, and solid tumor metastases to bone marrow.
3. A bone marrow model including: a first microenvironment including a hydrogel with mature bone cells; and a second microenvironment including a hydrogel with cells found in the hematopoietic niche of normal bone; wherein the first microenvironment and second microenvironment are maintained in a position where they can interact with a fluid medium.
4. A bone marrow model including: a first microenvironment including a mineralized hydrogel with mature bone cells; and a second microenvironment including a hydrogel with cells found in the hematopoietic niche of normal bone; wherein the first microenvironment and second microenvironment are maintained in a position where they can interact with a fluid medium.
5. A bone marrow model including: a first microenvironment including a hydrogel with immature bone cells; and a second microenvironment including a hydrogel with cells found in the hematopoietic niche of normal bone; wherein the first microenvironment and second microenvironment are maintained in a position where they can interact with a fluid medium.
6. A bone marrow model including: a first microenvironment including a mineralized hydrogel with immature bone cells; and a second microenvironment including a hydrogel with cells found in the hematopoietic niche of normal bone; wherein the first microenvironment and second microenvironment are maintained in a position where they can interact with a fluid medium.
7. A bone marrow model including: a first microenvironment including a hydrogel without cells or with mature bone cells and/or mesenchymal stem cells; and a second microenvironment including a non-mineralized hydrogel incorporating elements of the hematopoietic niche; wherein the first microenvironment is in contact with the second microenvironment.
8. A bone marrow model including: a first microenvironment including a mineralized hydrogel without cells or with mature bone cells and/or mesenchymal stem cells; and a second microenvironment including a non-mineralized hydrogel incorporating elements of the hematopoietic niche; wherein the first microenvironment is in contact with the second microenvironment.
9. A bone marrow model including: a first microenvironment including a hydrogel with mature bone cells; and a second microenvironment including: a hydrogel with cells found in a normal hematopoietic niche of normal bone marrow; or a hydrogel with cells associated with a diseased state in a hemopoietic niche.
10. A bone marrow model including: a first microenvironment including a mineralized hydrogel with mature bone cells; and a second microenvironment including: a hydrogel with cells found in a normal hematopoietic niche of normal bone marrow; or a hydrogel with cells associated with a diseased state in a hemopoietic niche.
11. A bone marrow model including: a first microenvironment including a hydrogel with immature bone cells and/or mesenchymal stem cells; and a second microenvironment including: a hydrogel with cells found in a normal hematopoietic niche of normal bone marrow; or a hydrogel with cells associated with a diseased state in a hemopoietic niche.
12. A bone marrow model including: a first microenvironment including a mineralized hydrogel with immature bone cells and/or mesenchymal stem cells; and a second microenvironment including: a hydrogel with cells found in a normal hematopoietic niche of normal bone marrow; or a hydrogel with cells associated with a diseased state in a hemopoietic niche.
13. The bone marrow model of any of embodiments 9, 10, 11, and 12, wherein the second microenvironment includes a hydrogel with cells found in a condition selected from the group consisting of hematologic malignancies, marrow failure syndromes, pre-malignant clonal hematopoiesis, and solid tumor metastases to bone marrow.
14. A bone marrow model including: a first microenvironment including a mineralized hydrogel without cells or with mature bone cells and/or mesenchymal stem cells; and a second microenvironment including a non-mineralized hydrogel incorporating elements of the hematopoietic niche; wherein the first microenvironment is in contact with the second microenvironment.
15. A microfluidic bone marrow model including: a central chamber open at two opposing ends and defined by continuous walls formed by an upper plate, a lower plate, and two side plates; wherein the upper plate and lower plate are substantially parallel to each other, are separated from one another by the height of the central chamber, and are substantially perpendicular to the two side plates; and wherein the two side plates are substantially parallel to each other, are separated from one another by the width of the central chamber, and are substantially perpendicular to the top plate and the bottom plate; and a permeable barrier dividing the central chamber parallel to and maintained between the upper plate and lower plate and in contact with each of the two side plates, the permeable barrier dividing the central chamber into an upper chamber located between the permeable barrier and upper plate and a lower central chamber located between the permeable barrier and the lower plate; wherein the lower central chamber is divided into a first microenvironment including an endosteal niche and a second microenvironment including a stem cell niche, the stem cell niche being in contact with the permeable barrier and the two side plates and the endosteal niche being in contact with the bottom plate and the two side plates; and further wherein the stem cell niche and the endosteal niche are in contact and communication with each other.
16. A bone marrow model including: a first microenvironment including a mineralized hydrogel without cells or with bone cells or mesenchymal stem cells; and is implemented as mineralized fragments in the form of shards; and a second microenvironment including a hydrogel with cells found in the hematopoietic niche of normal, modified, or diseased bone marrow; wherein the mineralized hydrogel of the first microenvironment is embedded in the hydrogel of the second microenvironment and the hydrogels of both the first and second microenvironments are maintained in a position where they can interact with a fluid medium.
17. A bone marrow model including: a) a first microenvironment including a mineralized hydrogel without cells or with mature bone cells or mesenchymal stem cells; wherein the mineralized hydrogel is in a form selected from the group of spheres, spheroids, beads, and droplets; and b) a second microenvironment including a hydrogel material with cells found in the hematopoietic niche of normal, modified, or diseased bone marrow; wherein the layers of hydrogel referenced above a) is embedded in b) and a) and b) are maintained in a position where they can interact with a fluid medium.
18. The bone marrow model of any of embodiments 2, 4, 6, 8, 10, 12, 14, 16, and 17, wherein the mineralized hydrogel of the first microenvironment is prepared by a method including: combining the living cells with a fluid medium and unlinked polymer chains to create a first composition; treating the first composition with a cross-linking agent or cross-linking treatment sufficient to cross-link the polymer chains to form a cross-linked polymer matrix including the living cells; and treating the cross-linked polymer matrix including the living cells with at least one mineralizing solution for a time sufficient to form the composition of a mineralized cross-linked polymer matrix including the living cells.
19. The bone marrow model of any of embodiments 2, 4, 6, 8, 10, 12, 14, 16, and 17, wherein the mineralized hydrogel of the first microenvironment is prepared by a method including: combining unlinked polymer chains with a fluid medium to create a first composition; treating the first composition with an agent or treatment sufficient to cross-link or entangle the polymer chains to form a polymer matrix; combining the living cells with the cross-linked polymer matrix to form a polymer matrix including the living cells; and treating the polymer matrix including the living cells with at least one mineralizing solution for a time sufficient to form the composition of a mineralized polymer matrix including the living cells.
20. The bone marrow model of any of embodiments 2, 4, 6, 8, 10, 12, 14, 16, and 17, wherein the mineralized hydrogel of the first microenvironment is prepared by a method including: combining the living cells with a fluid medium and acid solubilized Type 1 collagen chains to create a first composition; maintaining the first composition under conditions sufficient to cause the Type 1 collagen chains of the first composition to undergo fibrillogenesis and form a second composition including the living cells; and treating the second composition including the living cells with at least one mineralizing solution for a time sufficient to form the composition of a mineralized Type 1 collagen matrix including the living cells.
21. The bone marrow model of any of embodiments 2, 4, 6, 8, 10, 12, 14, 16, and 17, wherein the mineralized hydrogel of the first microenvironment is prepared by a method including: combining a fluid medium and acid solubilized Type 1 collagen chains to create a first composition; maintaining the first composition under conditions sufficient to cause the Type 1 collagen chains of the first composition to undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
22. The bone marrow model of any of embodiments 2, 4, 6, 8, 10, 12, 14, 16, and 17, wherein the mineralized hydrogel of the first microenvironment is prepared by a method including: combining the living cells with a fluid medium and acid solubilized Type 1 collagen chains to create a first composition; adjusting the pH of the first composition to from about 7.2 to about 7.6 and maintaining the first composition and maintaining the first composition at a pH of from about 7.2 to about 7.6 until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.
23. The bone marrow model of any of embodiments 2, 4, 6, 8, 10, 12, 14, 16, and 17, wherein the mineralized hydrogel of the first microenvironment is prepared by a method including: combining a fluid medium and acid solubilized Type 1 collagen chains to create a first composition; adjusting the pH of the first composition to from about 7.2 to about 7.6 and maintaining the first composition and maintaining the first composition at a pH of from about 7.2 to about 7.6 until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
24. The bone marrow model of any of embodiments 2, 4, 6, 8, 10, 12, 14, 16, and 17, wherein the mineralized hydrogel of the first microenvironment is prepared by a method including: combining the living cells with a fluid medium and acid solubilized Type 1 collagen chains to create a first composition; adjusting the pH of the first composition to from about 7.2 to about 7.6 and maintaining the first composition and maintaining the first composition at a pH of from about 7.2 to about 7.6 and a temperature of from about 34° C. to about 40° C. until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.
25. The bone marrow model of any of embodiments 2, 4, 6, 8, 10, 12, 14, 16, and 17, wherein the mineralized hydrogel of the first microenvironment is prepared by a method including: combining a fluid medium and acid solubilized Type 1 collagen chains to create a first composition; adjusting the pH of the first composition to from about 7.2 to about 7.6 and maintaining the first composition and maintaining the first composition at a pH of from about 7.2 to about 7.6 and a temperature of from about 34° C. to about 40° C. until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
26. The bone marrow model of any of embodiments 18, 19, 20, 21, 22, 23, 24, and 25, wherein the mineralizing solution includes from about 3.0 mM to about 6.0 mM of calcium ions.
27. The bone marrow model of any of embodiments 18, 19, 20, 21, 22, 23, 24, and 25, wherein the mineralizing solution includes from about 4.0 mM to about 5.0 mM of calcium ions.
28. The bone marrow model of any of embodiments 18, 19, 20, 21, 22, 23, 24, and 25, wherein the mineralizing solution includes from about 1.5 mM to about 3.0 mM of phosphate ions
29. The bone marrow model of any of embodiments 18, 19, 20, 21, 22, 23, 24, and 25, wherein the mineralizing solution includes from about 1.8 mM to about 2.5 mM of phosphate ions.
30. The bone marrow model of any of Embodiments 18, 19, 20, 21, 22, 23, 24, and 25, wherein the mineralizing solution includes from about 3.0 mM to about 6.0 mM of calcium ions and from about 1.5 mM to about 3.0 mM of phosphate ions.
31. The bone marrow model of any of embodiments 18, 19, 20, 21, 22, 23, 24, and 25, wherein the mineralizing solution includes from about 4.0 mM to about 5.0 mM of calcium ions and from about 1.8 mM to about 2.5 mM of phosphate ions.
32. The bone marrow model of any of embodiments 18, 19, 20, 21, 22, 23, 24, 25, 26, 27, 28, 29, 30, and 31, wherein the fluid medium includes at least one agent selected from the group of antibiotic agents, antifungal agents, antiviral agents, buffers, anticoagulants, vitamins, salts, minerals, amino acids, nucleic acids, ribonucleic acids, fatty acids, lipids, O2 gas, CO2 gas, carbohydrates, serum proteins, cofactors, growth factors, cytokines, enzymes, hormones, signaling substances, and antibodies.
33. The bone marrow model of any of Embodiments 18, 19, 20, 21, 22, 23, 24, 25, 26, 27, 28, 29, 30, 31, and 32, wherein the fluid medium includes from about 5× to about 20× phosphate buffered saline (PBS).
34. The bone marrow model of any of embodiments 18, 19, 20, 21, 22, 23, 24, 25, 26, 27, 28, 29, 30, 31, 32, and 33, wherein the fluid medium includes Dulbecco's Modified Eagle Medium (DMEM).
35. The bone marrow model of any of embodiments 18, 19, 20, 21, 22, 23, 24, 25, 26, 27, 28, 29, 30, 31, 32, 33, and 34, wherein the fluid medium includes from about 5× to about 20× phosphate buffered saline (PBS) and Dulbecco's Modified Eagle Medium (DMEM).
36. The bone marrow model of any of embodiments 18, 19, 20, 21, 22, 23, 24, 25, 26, 27, 28, 29, 30, 31, 32, 33, 34, and 35, wherein the at least one mineralizing solution includes an inhibitor of hydroxyapatite formation, nucleation, or precipitation during the mineralizing process.
37. The bone marrow model of embodiment 36, wherein the inhibitor of hydroxyapatite formation includes a protein capable of inhibiting hydroxyapatite nucleation and precipitation in solution.
38. The bone marrow model of embodiment 37, wherein the protein capable of inhibiting hydroxyapatite formation, nucleation, or precipitation is selected from the group consisting of Osteopontin (OPN), Osteocalcin (OC), Osteonectin (ON), bone sialoprotein (BSP), dentine phosphoryn (DPP), dentin matrix protein 1 (DMP1), dentin sialophosphoprotein (DSPP), matrix extracellularphosphoglycoprotein (MEPE), chondrocalcin (CC), Proline-rich protein 1 (PRP1), Proline-rich protein 2 (PRP2), Proline-rich protein 3 (PRP3), PRP1-T1, PRP3-T1, Histatin 5, MG1, MG2, Asialo_MG2, Amylase, statherin, cystatin S, cystatin SN, Cystatin S1, fetuin, and HSA.
39. The bone marrow model of any of embodiments 36, 37, and 38, wherein the inhibitor of hydroxyapatite formation, nucleation, or precipitation includes Osteopontin.
40. The bone marrow model of any of embodiments 18-39, wherein the mineralizing solution includes one or more sources of ionic minerals selected from the group of calcium phosphate, calcium carbonate, hydroxyapatite, strontium carbonate, barium carbonate, and calcium sulfate, strontium sulfate, calcium oxalate, magnesium-bearing calcium carbonate, and magnesium-bearing calcium phosphate.
41. The bone marrow model of any of embodiments 18-40, wherein the mineralizing solution is a calcifying solution.
42. The bone marrow model of embodiment 41, wherein the calcifying solution includes calcium ions and phosphate ions.
43. The bone marrow model of any of embodiments 1-42, further including one or more cell selected from the group consisting of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, and macrophages.
44. The bone marrow model of any of embodiments 1-43, wherein the second microenvironment includes stem cells.
45. The bone marrow model of any of embodiments 1-44, wherein the second microenvironment includes cells selected from the group consisting of hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, endothelial cells (ECs), osteoblast precursor cells, osteolineage cells, pericytes, chondrocyte precursor cells, mesenchymal stem and progenitor cells or mesenchymal stromal progenitor cells, and CXCL12-abundant reticular cells.
46. The bone marrow model of any of embodiments 1-45, wherein the first microenvironment includes one or more types of diseased cells.
47. The bone marrow model of any of embodiments 1-46, wherein the second microenvironment includes one or more types of diseased cells.
48. The bone marrow model of any of embodiments 1-47, wherein both the first microenvironment and second microenvironment include one or more types of diseased cells.
49. The bone marrow model of any of embodiments 1-48, wherein the second microenvironment includes cells found in hematopoietic niche experiencing or subject to a condition selected from the group consisting of hematologic malignancies, marrow failure syndromes, pre-malignant clonal hematopoiesis, and solid tumor metastases to bone marrow.
50. The bone marrow model of any of embodiments 44, 45, 46, 47, 48, and 49, wherein the one or more types of diseased cells are selected from the group consisting of acute myeloid leukemia, chronic myeloid leukemia, atypical chronic myeloid leukemia, chronic neutrophilic leukemia, acute lymphoblastic leukemia, multiple myeloma, Non-Hodgkin lymphoma, Chronic lymphocytic leukemia, Hodgkin lymphoma, T-cell lymphoma, bone marrow failure syndromes, myelodysplastic syndrome, clonal hematopoiesis of indeterminant potential, clonal cytopenias of undetermined significance, and aplastic anemia, and cells of metastatic solid tumors that travel to the bone marrow
51. The bone marrow model of embodiment 50 wherein the cells of metastatic solid tumors that travel to the bone marrow are selected from the group of lung cancers, breast cancers, kidney cancers, prostate cancers, and thyroid cancers.
52. The bone marrow model of any of embodiments 1-51, wherein the first microenvironment further includes cells selected from the group of osteoblasts, osteoprogenitors, and osteochondroprogenitors.
53. The bone marrow model of any of embodiments 1-52, wherein the first microenvironment further includes osteoblasts.
54. The bone marrow model of any of embodiments 1-53, wherein the second microenvironment includes cells selected from the group of mesenchymal stem cells and hematopoietic stem cells.
55. The bone marrow model of any of embodiments 1-54, wherein the first microenvironment includes a mineralized collagen.
56. The bone marrow model of any of embodiments 1-55, wherein the first microenvironment and the second microenvironment are maintained in a position in which they can interact with a fluid medium.
57. The bone marrow model of any of embodiments 1-56, further including one or more fluid channels in communication with at least one of the first microenvironment and the second microenvironment.
58. The bone marrow model of any of embodiments 1-57, further including a fluid channel in communication with the first microenvironment.
59. The bone marrow model of any of embodiments 1-57, further including a fluid channel in communication with the second microenvironment.
60. The bone marrow model of any of embodiments 1-57, further including a fluid channel in communication with both the first microenvironment and the second microenvironment.
61. The bone marrow model of any of embodiments 1-57, further including a first fluid channel and a second fluid channel, wherein the first fluid channel is in communication with both the first microenvironment and the second microenvironment and the second fluid channel is in communication with both the first microenvironment and the second microenvironment.
62. The bone marrow model of any of embodiments 1-57, further including a first fluid channel and a second fluid channel, wherein the first fluid channel is in communication with both the first microenvironment and the second microenvironment and the second fluid channel is in communication with one of the first microenvironment and the second microenvironment.
63. A method of testing a drug or drug candidate, or a pharmaceutically acceptable salt thereof, using the bone marrow model of any of embodiments 1-62, the method including: ascertaining the elements of a device or design described in the bone marrow model having first microenvironment and a second microenvironment; exposing at least one of the first microenvironment and the second microenvironment to the drug or drug candidate, or a pharmaceutically acceptable salt thereof; and ascertaining any changes to the elements of the device or design following exposure to the drug or drug candidate, or a pharmaceutically acceptable salt thereof.
64. The method of embodiment 63, wherein the at least one of the first microenvironment and the second microenvironment is exposed to the drug or drug candidate, or a pharmaceutically acceptable salt thereof, by contact with at least one fluid media including the drug or drug candidate, or a pharmaceutically acceptable salt thereof.
65. The method of embodiment 63 or embodiment 64, wherein the method of drug testing is accomplished in vivo.
66. The method of embodiment 65 wherein the drug testing is accomplished in vivo using a ring model as described herein.
67. The method of embodiment 63 or embodiment 64, wherein the method of drug testing is accomplished using a high-throughput static assay.
68. The method of embodiment 63 or embodiment 64, wherein the method of drug testing is accomplished using high-throughput flow cytometry.

### Second Set of Representative Embodiments

1. A bone marrow model including: a first microenvironment including a hydrogel with or without cells; and a second microenvironment including: a hydrogel with cells found in a normal hematopoietic niche of normal bone marrow; or a hydrogel with cells found in a condition selected from the group consisting of hematologic malignancies, marrow failure syndromes, pre-malignant clonal hematopoiesis, and solid tumor metastases to bone marrow.
2. The bone marrow model of embodiment 1, wherein the first microenvironment includes a mineralized hydrogel.
3. A bone marrow model including: a first microenvironment including a hydrogel with mature bone cells; and a second microenvironment including a hydrogel with cells found in a hematopoietic niche of normal bone; wherein the first microenvironment and second microenvironment are maintained in a position where they can interact with a fluid medium.
4. The bone marrow model of embodiment 3, wherein the first microenvironment includes a mineralized hydrogel.
5. A bone marrow model including: a first microenvironment including a hydrogel with immature bone cells; and a second microenvironment including a hydrogel with cells found in a hematopoietic niche of normal bone; wherein the first microenvironment and second microenvironment are maintained in a position where they can interact with a fluid medium.
6. The bone marrow model of embodiment 5, wherein the first microenvironment includes a mineralized hydrogel.
7. A bone marrow model including: a first microenvironment including a hydrogel without cells or with mature bone cells and/or mesenchymal stem cells; and a second microenvironment including a non-mineralized hydrogel incorporating elements of a hematopoietic niche; wherein the first microenvironment is in contact with the second microenvironment.
8. The bone marrow model of embodiment 7, wherein the first microenvironment includes a mineralized hydrogel.
9. A bone marrow model including: a first microenvironment including a hydrogel with mature bone cells; and a second microenvironment including: a hydrogel with cells found in a normal hematopoietic niche of normal bone marrow; or a hydrogel with cells associated with a diseased state in a hemopoietic niche.
10. The bone marrow model of embodiment 9, wherein the first microenvironment including a mineralized hydrogel.
11. A bone marrow model including: a first microenvironment including a hydrogel with immature bone cells and/or mesenchymal stem cells; and a second microenvironment including: a hydrogel with cells found in a normal hematopoietic niche of normal bone marrow; or a hydrogel with cells associated with a diseased state in a hemopoietic niche.
12. The bone marrow model of embodiment 11, wherein the first microenvironment including a mineralized hydrogel.
13. The bone marrow model of any of embodiments 9, 10, 11, or 12, wherein the second microenvironment includes a hydrogel with cells found in a condition selected from the group consisting of hematologic malignancies, marrow failure syndromes, pre-malignant clonal hematopoiesis, and solid tumor metastases to bone marrow.
14. A bone marrow model including: a first microenvironment including a mineralized hydrogel without cells or with mature bone cells and/or mesenchymal stem cells; and a second microenvironment including a non-mineralized hydrogel incorporating elements of a hematopoietic niche; wherein the first microenvironment is in contact with the second microenvironment.
16. A bone marrow model including: a first microenvironment including a mineralized hydrogel without cells or with bone cells or mesenchymal stem cells; and is implemented as mineralized fragments in the form of shards; and a second microenvironment including a hydrogel with cells found in the hematopoietic niche of normal, modified, or diseased bone marrow; wherein the mineralized hydrogel of the first microenvironment is embedded in the hydrogel of the second microenvironment and the hydrogels of both the first and second microenvironment s are maintained in a position where they can interact with a fluid medium.
17. A bone marrow model including: a) a first microenvironment including a mineralized hydrogel without cells or with mature bone cells or mesenchymal stem cells; and b) a second microenvironment including a hydrogel material with cells found in the hematopoietic niche of normal, modified, or diseased bone marrow; wherein the layers of hydrogel referenced above a) is embedded in b) and a) and b) are maintained in a position where they can interact with a fluid medium.
18. The bone marrow model of embodiment 17, wherein the mineralized hydrogel is in a form spheres, spheroids, beads, and/or droplets.
19. The bone marrow model of any of embodiments 2, 4, 6, 8, 10, 12, 14, 16, 17, and 18 wherein the mineralized hydrogel of the first microenvironment is prepared by a method including: combining the living cells with a fluid medium and unlinked polymer chains to create a first composition; treating the first composition with a cross-linking agent or cross-linking treatment sufficient to cross-link the polymer chains to form a cross-linked polymer matrix including the living cells; and treating the cross-linked polymer matrix including the living cells with at least one mineralizing solution for a time sufficient to form the composition of a mineralized cross-linked polymer matrix including the living cells.
20. The bone marrow model of any of embodiments 2, 4, 6, 8, 10, 12, 14, 16, 17, and 18, wherein the mineralized hydrogel of the first microenvironment is prepared by a method including: combining unlinked polymer chains with a fluid medium to create a first composition; treating the first composition with an agent or treatment sufficient to cross-link or entangle the polymer chains to form a polymer matrix; combining the living cells with the cross-linked polymer matrix to form a polymer matrix including the living cells; and treating the polymer matrix including the living cells with at least one mineralizing solution for a time sufficient to form the composition of a mineralized polymer matrix including the living cells.
21. The bone marrow model of any of embodiments 2, 4, 6, 8, 10, 12, 14, 16, 17, and 18, wherein the mineralized hydrogel of the first microenvironment is prepared by a method including: combining the living cells with a fluid medium and acid solubilized Type 1 collagen chains to create a first composition; maintaining the first composition under conditions sufficient to cause the Type 1 collagen chains of the first composition to undergo fibrillogenesis and form a second composition including the living cells; and treating the second composition including the living cells with at least one mineralizing solution for a time sufficient to form the composition of a mineralized Type 1 collagen matrix including the living cells.
22. The bone marrow model of any of embodiments 2, 4, 6, 8, 10, 12, 14, 16, 17, and 18, wherein the mineralized hydrogel of the first microenvironment is prepared by a method including: combining a fluid medium and acid solubilized Type 1 collagen chains to create a first composition; maintaining the first composition under conditions sufficient to cause the Type 1 collagen chains of the first composition to undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
23. The bone marrow model of any of embodiments 2, 4, 6, 8, 10, 12, 14, 16, 17, and 8, wherein the mineralized hydrogel of the first microenvironment is prepared by a method including: combining the living cells with a fluid medium and acid solubilized Type 1 collagen chains to create a first composition; adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.
24. The bone marrow model of any of embodiments 2, 4, 6, 8, 10, 12, 14, 16, 17, and 18 wherein the mineralized hydrogel of the first microenvironment is prepared by a method including: combining a fluid medium and acid solubilized Type 1 collagen chains to create a first composition; adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
25. The bone marrow model of any of embodiments 2, 4, 6, 8, 10, 12, 14, 16, 17, and 18, wherein the mineralized hydrogel of the first microenvironment is prepared by a method including: combining the living cells with a fluid medium and acid solubilized Type 1 collagen chains to create a first composition; adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 and a temperature of from about 34° C. to about 40° C. until the Type 1 collagen chains undergo fibrillogenesis and form a second composition including the living cells; and treating the second composition including the living cells with at least one mineralizing solution for a time sufficient to form the composition of a mineralized Type 1 collagen matrix including the living cells.
26. The bone marrow model of any of embodiments 2, 4, 6, 8, 10, 12, 14, 16, 17, and 18, wherein the mineralized hydrogel of the first microenvironment is prepared by a method including: combining a fluid medium and acid solubilized Type 1 collagen chains to create a first composition; adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 and a temperature of from about 34° C. to about 40° C. until the Type 1 collagen chains undergo fibrillogenesis and form a second composition; combining the living cells with the second composition to form a third composition including the living cells; and treating the third composition including the living cells with at least one mineralizing solution for a time sufficient to form the composition of a mineralized Type 1 collagen matrix including the living cells.
27. The bone marrow model of any of embodiments 18-26, wherein the mineralizing solution includes: from about 3.0 mM to about 6.0 mM of calcium ions; or from about 4.0 mM to about 5.0 mM of calcium ions.
28. The bone marrow model of any of embodiments 18-26, wherein the mineralizing solution includes: from about 1.5 mM to about 3.0 mM of phosphate ions; or from about 1.8 mM to about 2.5 mM of phosphate ions.
29. The bone marrow model of any of embodiments 18-26, wherein the mineralizing solution includes: from about 3.0 mM to about 6.0 mM of calcium ions and from about 1.5 mM to about 3.0 mM of phosphate ions; or from about 4.0 mM to about 5.0 mM of calcium ions and from about 1.8 mM to about 2.5 mM of phosphate ions.
30. The bone marrow model of any of embodiments 18-29, wherein the fluid medium includes at least one agent selected from the group of antibiotic agents, antifungal agents, antiviral agents, buffers, anticoagulants, vitamins, salts, minerals, amino acids, nucleic acids, ribonucleic acids, fatty acids, lipids, O₂ gas, CO₂ gas, carbohydrates, serum proteins, cofactors, growth factors, cytokines, enzymes, hormones, signaling substances, and antibodies.
31. The bone marrow model of any of embodiments 18-30, wherein the fluid medium includes from about 1× to about 20× phosphate buffered saline (PBS).
32. The bone marrow model of any of embodiments 18-31, wherein the fluid medium includes Dulbecco's Modified Eagle Medium (DMEM).
33. The bone marrow model of any of embodiments 18-32, wherein the fluid medium includes from about 5× to about 20× phosphate buffered saline (PBS) and Dulbecco's Modified Eagle Medium (DMEM).
34. The bone marrow model of any of embodiments 18-33, wherein the at least one mineralizing solution includes an inhibitor of hydroxyapatite formation, nucleation, or precipitation during the mineralizing process.
35. The bone marrow model of embodiment 34, wherein the inhibitor of hydroxyapatite formation includes a protein capable of inhibiting hydroxyapatite nucleation and precipitation in solution.
36. The bone marrow model of embodiment 35, wherein the protein capable of inhibiting hydroxyapatite formation, nucleation, or precipitation is selected from the group consisting of Osteopontin (OPN), Osteocalcin (OC), Osteonectin (ON), bone sialoprotein (BSP), dentine phosphoryn (DPP), dentin matrix protein 1 (DMP1), dentin sialophosphoprotein (DSPP), matrix extracellularphosphoglycoprotein (MEPE), chondrocalcin (CC), Proline-rich protein 1 (PRP1), Proline-rich protein 2 (PRP2), Proline-rich protein 3 (PRP3), PRP1-T1, PRP3-T1, Histatin 5, MG1, MG2, Asialo_MG2, Amylase, statherin, cystatin S, cystatin SN, Cystatin S1, fetuin, and HSA.
37. The bone marrow model of any of embodiments 34-36, wherein the inhibitor of hydroxyapatite formation, nucleation, or precipitation includes Osteopontin.
38. The bone marrow model of embodiment 37, wherein the osteopontin is present at a concentration of 50 μg/mL to 200 μg/mL.
39. The bone marrow model of embodiment 38, wherein the osteopontin is present at a concentration of 100 μg/mL.
40. The bone marrow model of any of embodiments 18-39, wherein the mineralizing solution includes one or more sources of ionic minerals selected from the group of calcium phosphate, calcium carbonate, hydroxyapatite, strontium carbonate, barium carbonate, and calcium sulfate, strontium sulfate, calcium oxalate, magnesium-bearing calcium carbonate, and magnesium-bearing calcium phosphate.
41. The bone marrow model of any of embodiments 18-40, wherein the mineralizing solution is a calcifying solution.
42. The bone marrow model of embodiment 41, wherein the calcifying solution includes calcium ions and phosphate ions.
43. A microfluidic bone marrow model including: a central chamber open at two opposing ends and defined by continuous walls formed by an upper plate, a lower plate, and two side plates; wherein the upper plate and lower plate are substantially parallel to each other, are separated from one another by the height of the central chamber, and are substantially perpendicular to the two side plates; and wherein the two side plates are substantially parallel to each other, are separated from one another by the width of the central chamber, and are substantially perpendicular to the top plate and the bottom plate; and a permeable barrier dividing the central chamber parallel to and maintained between the upper plate and lower plate and in contact with each of the two side plates, the permeable barrier dividing the central chamber into an upper chamber located between the permeable barrier and upper plate and a lower central chamber located between the permeable barrier and the lower plate; wherein the lower central chamber is divided into a first microenvironment including an endosteal niche and a second microenvironment including a stem cell niche, the stem cell niche being in contact with the permeable barrier and the two side plates and the endosteal niche being in contact with the bottom plate and the two side plates; and further wherein the stem cell niche and the endosteal niche are in contact and communication with each other.
44. The bone marrow model of any of embodiments 1-43, further including one or more cell selected from the group consisting of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages, and exosomes.
45. The bone marrow model of any of embodiments 1-44, wherein the second microenvironment includes stem cells.
46. The bone marrow model of any of embodiments 1-45, wherein the second microenvironment includes one or more of hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, endothelial cells (ECs), osteoblast precursor cells, osteolineage cells, pericytes, chondrocyte precursor cells, mesenchymal stem and progenitor cells or mesenchymal stromal progenitor cells, CXCL12-abundant reticular cells, and exosomes.
47. The bone marrow model of any of embodiments 1-46, wherein: the first microenvironment includes one or more types of diseased cells; the second microenvironment includes one or more types of diseased cells; or both the first microenvironment and second microenvironment include one or more types of diseased cells.
48. The bone marrow model of any of embodiments 1-47, wherein the second microenvironment includes cells found in hematopoietic niche experiencing or subject to a condition selected from the group consisting of hematologic malignancies, marrow failure syndromes, pre-malignant clonal hematopoiesis, and solid tumor metastases to bone marrow.
49. The bone marrow model of any of embodiments 45-48, wherein the one or more types of diseased cells are selected from acute myeloid leukemia, chronic myeloid leukemia, atypical chronic myeloid leukemia, chronic neutrophilic leukemia, acute lymphoblastic leukemia, multiple myeloma, Non-Hodgkin lymphoma, Chronic lymphocytic leukemia, Hodgkin lymphoma, T-cell lymphoma, bone marrow failure syndromes, myelodysplastic syndrome, clonal hematopoiesis of indeterminant potential, clonal cytopenias of undetermined significance, and aplastic anemia, and circulating tumor cells and cells of metastatic solid tumors that travel to the bone marrow
50. The bone marrow model of embodiment 49 wherein the cells of metastatic solid tumors that travel to the bone marrow are selected from the group of lung cancers, breast cancers, kidney cancers, prostate cancers, and thyroid cancers.
51. The bone marrow model of any of embodiments 1-50, wherein the first microenvironment further includes cells selected from the group of osteoblasts, osteoprogenitors, and osteochondroprogenitors.
52. The bone marrow model of any of embodiments 1-51, wherein the first microenvironment further includes osteoblasts.
53. The bone marrow model of any of embodiments 1-52, wherein the second microenvironment includes cells selected from the group of mesenchymal stem cells and hematopoietic stem cells.
54. The bone marrow model of any of embodiments 1-53, wherein the first microenvironment includes a mineralized collagen.
55. The bone marrow model of any of embodiments 1-54, wherein the first microenvironment and the second microenvironment are maintained in a position in which they can interact with a fluid medium.
56. The bone marrow model of any of embodiments 1-55, further including one or more fluid channels in communication with at least one of the first microenvironment and the second microenvironment.
57. The bone marrow model of any of embodiments 1-56, further including a fluid channel in communication with the first microenvironment.
58. The bone marrow model of any of embodiments 1-56, further including a fluid channel in communication with the second microenvironment.
59. The bone marrow model of any of embodiments 1-56, further including a fluid channel in communication with both the first microenvironment and the second microenvironment.
60. The bone marrow model of any of embodiments 1-56, further including a first fluid channel and a second fluid channel, wherein the first fluid channel is in communication with both the first microenvironment and the second microenvironment and the second fluid channel is in communication with both the first microenvironment and the second microenvironment.
61. The bone marrow model of any of embodiments 1-56, further including a first fluid channel and a second fluid channel, wherein the first fluid channel is in communication with both the first microenvironment and the second microenvironment and the second fluid channel is in communication with one of the first microenvironment and the second microenvironment.
62. The bone marrow model of any one of embodiments 57-61, wherein epithelial cells line at least a portion of the first fluid channel, the second fluid channel, or both the first and the second fluid channels.
63. The bone marrow model of any one of embodiments 57-61, wherein the vascular niche hydrogel contains endothelial cells.
64. A method of testing a drug or drug candidate, or a pharmaceutically acceptable salt thereof, using the bone marrow model of any of embodiments 1-63, the method including: ascertaining the elements of a device or design described in the bone marrow model having first microenvironment and a second microenvironment; exposing at least one of the first microenvironment and the second microenvironment to the drug or drug candidate, or a pharmaceutically acceptable salt thereof; and ascertaining any changes to the elements of the device or design following exposure to the drug or drug candidate, or a pharmaceutically acceptable salt thereof.
65. The method of embodiment 65, wherein the at least one of the first microenvironment and the second microenvironment is exposed to the drug or drug candidate, or a pharmaceutically acceptable salt thereof, by contact with at least one fluid media including the drug or drug candidate, or a pharmaceutically acceptable salt thereof.
66. The method of embodiment 64 or embodiment 65, wherein the method of drug testing is accomplished in vivo.
67. The method of embodiment 66 wherein the drug testing is accomplished in vivo using a ring, disk, or shard model.
68. The method of embodiment 64 or embodiment 65, wherein the drug testing is accomplished using one or more of a high-throughput static assay; a high-throughput flow assay; or a high-throughput microfluidic device.
69. A system for use in a bone marrow model, including: a well plate including: an array of media wells configured to receive fluid media, a media well of the array of media wells including a bottom end that is covered with a permeable barrier; an array of hydrogel chambers, a hydrogel chamber of the array of hydrogel chambers positioned underneath the permeable barrier; and an array of loading ports, a loading port of the array of loading ports in fluid communication with the hydrogel chamber to direct a hydrogel containing cells into the hydrogel chamber.
70. The system of embodiment 69, wherein the loading port is positioned beside the media well, and wherein the hydrogel chamber is positioned underneath the loading port.
71. The system of embodiment 69, wherein a first number of the array of loading ports is greater than a second number of the array of media wells, wherein the loading port is a first loading port, and wherein a second loading port of the array of loading ports is in fluid communication with the hydrogel chamber.
72. The system of embodiment 69, wherein the loading port has a first end configured to receive a pipette tip and a second end at an opening to the hydrogel chamber, wherein the first end of the loading port has a first inner diameter, and wherein the second end of the loading port has a second inner diameter less than the first inner diameter.
73. The system of embodiment 69, wherein the well plate is a first well plate, wherein the media well further includes a top end that opens into a recessed area at a top of the first well plate, wherein the first well plate further includes a connector on an external side surface of the first well plate, and wherein the system further includes a second well plate configured to detachably couple to the top of the first well plate, the second well plate including:
an array of tubes extending from a bottom surface of the second well plate, a tube of the array of tubes having an outer diameter less than an inner diameter of the media well to allow the tube to be inserted into the media well when the second well plate is coupled to the top of the first well plate; and an array of collection wells in fluid communication with the array of tubes, a collection well of the array of collection wells positioned over the media well when the second well plate is coupled to the top of the first well plate, wherein the recessed area at the top of the first well plate forms an air chamber when the second well plate is coupled to the top of the first well plate, and wherein the air chamber is configured to be pressurized through operation of a pump that is connected to the connector.
74. The system of embodiment 73, wherein at least one of the first well plate or the second well plate includes an elastomer to create a hermetic seal when the second well plate is coupled to the top of the first well plate.
75. The system of embodiment 73, wherein the array of media wells are arranged in rows, wherein a row of the rows includes multiple media wells, wherein each media well of the multiple media wells in the row is positioned adjacent to at least one loading port of the array of loading ports, wherein the row is separated from an adjacent row by a vertically-oriented wall to define the air chamber for the row of the multiple media wells, and wherein the connector allows for pressurizing the air chamber independently from other air chambers of other rows.
76. A system for use in a bone marrow model, including: a well plate including: a first media well and a second media well, the first media well and the second media well configured to receive fluid media, wherein a first bottom end of the first media well is covered with a permeable membrane, and wherein a second bottom end of the second media well is covered with the permeable membrane, or a different permeable membrane; a first extracellular matrix (ECM) chamber and a second ECM chamber, wherein the first ECM chamber is positioned underneath the first media well with the permeable membrane interposed between the first ECM chamber and the first media well, and wherein the second ECM chamber is positioned underneath the second media well with the permeable membrane, or the different permeable membrane, interposed between the second ECM chamber and the second media well; and a first loading port and a second loading port, wherein the first loading port is in fluid communication with the first ECM chamber, and wherein the second loading port is in fluid communication with the second ECM chamber.
77. The system of embodiment 76, wherein the first loading port is positioned beside the first media well, the first ECM chamber is positioned underneath the first loading port, the second loading port is beside the second media well, and the second ECM chamber is positioned underneath the second loading port.
78. The system of embodiment 76, wherein the well plate further includes a third loading port and a fourth loading port, wherein the third loading port is in fluid communication with the first ECM chamber, and wherein the fourth loading port is in fluid communication with the second ECM chamber.
79. The system of embodiment 76, wherein the first loading port has a first end configured to receive a pipette tip and a second end at an opening to the first ECM chamber, wherein the first end of the first loading port has a first inner diameter, and wherein the second end of the first loading port has a second inner diameter less than the first inner diameter.
80. The system of embodiment 76, wherein the well plate is a first well plate, wherein the first media well includes a first top end that opens into a first recessed area at a top of the first well plate, wherein the second media well includes a second top end that opens into a second recessed area at the top of the first well plate, wherein a vertically-oriented wall separates the first recessed area from the second recessed area, wherein the first well plate further includes a connector and a second connector on an external side surface of the first well plate, and wherein the system further includes a second well plate configured to detachably couple to the top of the first well plate, the second well plate including: a first tube and a second tube extending from a bottom surface of the second well plate, wherein the first tube is configured to be inserted into the first media well and the second tube is configured to be inserted into the second media well when the second well plate is coupled to the top of the first well plate; a first collection well in fluid communication with the first tube and positioned over the first media well when the second well plate is coupled to the top of the first well plate; and a second collection well in fluid communication with the second tube and positioned over the second media well when the second well plate is coupled to the top of the first well plate, wherein the first recessed area forms a first air chamber and the second recessed area forms a second air chamber when the second well plate is coupled to the top of the first well plate, and wherein the first air chamber is configured to be pressurized through operation of a pump that is connected to the first connector and the second air chamber is configured to be pressurized through operation of the pump, or a different pump, that is connect to the second connector.
81. The system of embodiment 80, wherein at least one of the first well plate or the second well plate includes an elastomer to create a hermetic seal when the second well plate is coupled to the top of the first well plate.
82. A method including: expressing, into an array of loading ports in a well plate, a hydrogel containing cells to at least partially fill an array of hydrogel chambers in the well plate with the hydrogel; and filling, at least partially, an array of media wells in the well plate with fluid media, wherein a media well of the array of media wells includes a bottom end that is covered with a permeable barrier, wherein a hydrogel chamber of the array of hydrogel chambers is positioned underneath the permeable barrier, and wherein the cells include one or more of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages, hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, osteoblast precursor cells, osteolineage cells, chondrocyte precursor cells, mesenchymal stem and progenitor cells or mesenchymal stromal progenitor cells, CXCL12-abundant reticular cells, and exosomes.
83. The method of embodiment 82, further including: mounting the well plate to an orbital shaker; and operating the orbital shaker to agitate the fluid media and/or the hydrogel.
84. The method of embodiment 82, wherein the expressing the hydrogel to at least partially fill the hydrogel chamber includes expressing the hydrogel from a pipette containing the hydrogel while a tip of the pipette is inserted into a loading port of the array of loading ports that is in fluid communication with the hydrogel chamber.
85. The method of embodiment 82, wherein the well plate is a first well plate, wherein a recessed area is defined in a top of the first well plate above one or more media wells of the array of media wells, and wherein the method further includes coupling a second well plate to the top of the first well plate to convert the recessed area into a hermetically-sealed air chamber; coupling a pump to a connector on an external side surface of the first well plate; and operating the pump to pressurize the hermetically-sealed air chamber as a pressurized air chamber, wherein the second well plate includes an array of tubes extending from a bottom surface of the second well plate; wherein tubes in the array of tubes are in fluid communication with collection wells in an array of collection wells in the second well plate; wherein one or more tubes of the array of tubes are inserted into the one or more media wells as a result of the coupling of the second well plate to the top of the first well plate; and wherein the pressurized air chamber causes continuous perfusion of the fluid media through the one or more tubes.
86. The method of embodiment 85, wherein the pressurized air chamber is pressurized with a positive pressure or a negative pressure.
87. The method of embodiment 85, wherein the pressurized air chamber causes the fluid media to flow in an upward direction through the one or more tubes and into one or more collection wells of the array of collection wells.
88. The method of embodiment 82, wherein the well plate is a first well plate, wherein multiple recessed areas are defined in a top of the first well plate, the multiple recessed areas including a first recessed area above a first set of media wells of the array of media wells and a second recessed area above a second set of media wells of the array of media wells, and wherein the method further includes coupling a second well plate to the top of the first well plate to convert the first recessed area into a first hermetically-sealed air chamber and the second recessed area into a second hermetically-sealed air chamber; coupling one or more pumps to a first connector and a second connector on an external side surface of the first well plate; and operating the one or more pumps to pressurize the first hermetically-sealed air chamber as a first pressurized air chamber and the second hermetically-sealed as a second pressurized air chamber, wherein the second well plate includes an array of tubes extending from a bottom surface of the second well plate; wherein tubes in the array of tubes are in fluid communication with collection wells in an array of collection wells in the second well plate; wherein a first set of tubes of the array of tubes are inserted into the first set of media wells and a second set of tubes of the array of tubes are inserted into the second set of media wells as a result of the coupling of the second well plate to the top of the first well plate; and wherein the first pressurized air chamber causes continuous perfusion of the fluid media through the first set of tubes and the second pressurized air chamber causes continuous perfusion of the fluid media through the second set of tubes.
89. A system for use in a bone marrow model, including: a first reservoir including: an array of microfluidic channels, a microfluidic channel of the array of microfluidic channels configured to receive fluid media at an inlet of the microfluidic channel and allow the fluid media to egress from an outlet of the microfluidic channel; and an array of inserts, an insert of the array of inserts coupled to at least one of the inlet of the microfluidic channel or the outlet of the microfluidic channel and including: a media channel to receive the fluid media at an inlet of the media channel and allow the fluid media to egress from an outlet of the media channel; a hydrogel chamber positioned underneath the media channel, the hydrogel chamber configured to be filled, at least partially, with a hydrogel containing cells; and a permeable barrier interposed between the hydrogel chamber and the media channel; and a second reservoir configured to detachably couple to a bottom of the first reservoir, the second reservoir including: an array of collection wells, a collection well of the array of collection wells positioned underneath the microfluidic channel when the second reservoir is coupled to the bottom of the first reservoir and configured to collect the fluid media that has passed through the microfluidic channel and through the insert.
90. The system of embodiment 89, wherein the first reservoir further includes a recessed area defined in a top of the first reservoir to receive the fluid media, and wherein the inlet of the microfluidic channel is positioned at a bottom of the recessed area.
91. The system of embodiment 89, wherein the array of microfluidic channels includes an array of spiral channels.
92. The system of embodiment 91, wherein an individual spiral channel of the array of spiral channels has: a length that is no greater than about 11 millimeters; and a diameter that is no greater than about 11 millimeters.
93. The system of embodiment 89, wherein the array of microfluidic channels includes an array of serpentine channels.
94. The system of embodiment 93, wherein an individual serpentine channel of the array of serpentine channels has a width that is no greater than about 8 millimeters.
95. The system of embodiment 89, wherein:
the second reservoir further includes a recessed section at a periphery of a top of the second reservoir; the first reservoir further includes a lip around a periphery of the bottom of the first reservoir; and the second reservoir is configured to detachably couple to the bottom of the first reservoir by inserting the lip into the recessed section.
96. The system of embodiment 89, wherein the inlet of the media channel is positioned at a top of the insert at a first side of the insert, and wherein the outlet of the media channel is positioned at a bottom of the insert at a second side of the insert opposite the first side.
97. A system for use in a bone marrow model, including: a first reservoir including: a first microfluidic channel and a second microfluidic channel, the first microfluidic channel and the second microfluidic channel each configured to allow fluid media to pass therethrough; and a first insert and a second insert, the first insert coupled to at least one of an inlet of the first microfluidic channel or an outlet of the first microfluidic channel, and the second insert coupled to at least one of an inlet of the second microfluidic channel or an outlet of the second microfluidic channel, the first insert and the second insert each including: a media channel to receive the fluid media at an inlet of the media channel and allow the fluid media to egress from an outlet of the media channel; an extracellular matrix (ECM) chamber positioned underneath the media channel, the ECM chamber configured to be filled, at least partially, with an ECM; and a permeable barrier interposed between the ECM chamber and the media channel; and a second reservoir including: a first collection well and a second collection well, wherein the first collection well is vertically-aligned with the first microfluidic channel and the second collection well is vertically-aligned with the second microfluidic channel when the second reservoir is positioned underneath the first reservoir.
98. The system of embodiment 97, wherein the first reservoir further includes a recessed area defined in a top of the first reservoir to receive the fluid media, and wherein the inlet of the first microfluidic channel and the inlet of the second microfluidic channel are positioned at a bottom of the recessed area.
99. The system of embodiment 97, wherein at least one of the first microfluidic channel or the second microfluidic channel includes a spiral channel.
100. The system of embodiment 99, wherein the spiral channel has: a length that is no greater than about 11 millimeters; and a diameter that is no greater than about 11 millimeters.
101. The system of embodiment 97, wherein at least one of the first microfluidic channel or the second microfluidic channel includes a serpentine channel.
102. The system of embodiment 101, wherein the serpentine channel has a width that is no greater than about 8 millimeters.
103. The system of embodiment 97, wherein: the second reservoir further includes a recessed section at a periphery of a top of the second reservoir; and
the first reservoir further includes a lip around a periphery of the bottom of the first reservoir, the lip configured to be inserted into the recessed section when the first reservoir is set on top of the second reservoir.
104. The system of embodiment 97, wherein the inlet of the media channel is positioned at a top of the first insert and on a first side of the first insert, and wherein the outlet of the media channel is positioned at a bottom of the first insert and on a second side of the first insert, the second side opposite the first side.
105. A vacuum insert for use in a bone marrow model, including: an annular base including: a plurality of inlets defined in a top of the annular base; a media channel defined in an interior of the annular base and in fluid communication with the plurality of inlets; and a permeable barrier positioned underneath the media channel; and a central tube coupled to the annular base at a center of the annular base and extending orthogonally from the top of the annular base, wherein the vacuum insert is configured to couple to a vacuum source at a top end of the central tube, and wherein the media channel of the annular base is configured to allow fluid media drawn into the vacuum insert via the plurality of inlets to pass through the annular base and into the central tube.
106. The vacuum insert of embodiment 105, wherein the vacuum insert is configured to be placed inside a media well of a well plate with the annular base at a bottom of the media well.
107. The vacuum insert of embodiment 105, wherein the permeable barrier spans an area of the annular base.
108. The vacuum insert of embodiment 105, wherein the annular base includes a recessed area defined in a bottom of the annular base to accommodate a hydrogel containing cells underneath the permeable barrier.
109. The vacuum insert of embodiment 105, wherein the annular base further includes a hydrogel chamber defined in the annular base underneath the permeable barrier, the hydrogel chamber configured to be loaded with hydrogel containing cells.
110. The vacuum insert of embodiment 105, wherein the plurality of inlets are spaced equidistantly around the periphery of the annular base.
111. A vacuum insert for use in a bone marrow model, including: an annular base including: a one or more inlets defined in a top of the annular base; a media channel defined in an interior of the annular base and in fluid communication with the one or more inlets; and a permeable barrier positioned underneath the media channel; and a central tube at a center of the annular base and extending orthogonally from the top of the annular base, wherein the vacuum insert is configured to couple to a vacuum source at a top end of the central tube, and wherein the central tube is in fluid communication with the media channel.
112. The vacuum insert of embodiment 111, wherein the vacuum insert is configured to be placed inside a media well of a well plate.
113. The vacuum insert of embodiment 112, wherein the vacuum insert is configured to be aspirated while other vacuum inserts within other media wells of the well plate are being aspirated via the vacuum source.
114. The vacuum insert of embodiment 111, wherein the permeable barrier spans an area of the annular base.
115. The vacuum insert of embodiment 111, wherein the annular base further includes a recessed area defined in a bottom of the annular base to accommodate a hydrogel containing cells underneath the permeable barrier.
115. The vacuum insert of embodiment 111, wherein the annular base further includes a hydrogel chamber defined in the annular base underneath the permeable barrier, the hydrogel chamber configured to be loaded with hydrogel containing cells.
116. The vacuum insert of embodiment 111, wherein the one or more inlets include a plurality of inlets spaced equidistantly around the periphery of the annular base.
117. A microfluidic chip for use in a bone marrow model, including: a first substrate including: an inlet defined in a top of the first substrate, the inlet configured to allow fluid media to ingress into the microfluidic chip; an outlet defined in the top of the first substrate, the outlet configured to allow the fluid media to egress from the microfluidic chip; and a loading port defined in the top of the first substrate, the loading port configured to receive a hydrogel containing cells; a second substrate disposed underneath the first substrate, the second substrate including: a hydrogel channel configured to be filled, at least partially, with the hydrogel containing cells, wherein an end of the hydrogel channel is vertically-aligned with the loading port; a first through-hole that is vertically-aligned with the inlet; and a second through-hole that is vertically-aligned with the outlet; a permeable barrier disposed underneath the second substrate; and a third substrate disposed underneath the permeable barrier, the third substrate including: a media channel configured to receive the fluid media, wherein a first end of the media channel is vertically-aligned with the first through-hole and a second end of the media channel is vertically-aligned with the second through-hole.
118. The microfluidic chip of embodiment 117, wherein the media channel spans a center of the third substrate and is oriented horizontally on the third substrate.
119. The microfluidic chip of embodiment 117, wherein the hydrogel channel spans a center of the second substrate and is oriented horizontally on the second substrate.
120. The microfluidic chip of embodiment 117, wherein: a center portion of the hydrogel channel is straight; and a peripheral portion of the hydrogel channel is curved.
121. The microfluidic chip of embodiment 117, wherein: the first substrate further includes a second loading port defined in the top of the first substrate; and the hydrogel channel includes a second end that is vertically-aligned with the second loading port.
122. The microfluidic chip of embodiment 117, wherein: the first substrate further includes: a second inlet defined in the top of the first substrate, the second inlet configured to allow the fluid media to ingress into the microfluidic chip; a second outlet defined in the top of the first substrate, the second outlet configured to allow the fluid media to egress from the microfluidic chip; and a second loading port defined in the top of the first substrate, the second loading port configured to receive the hydrogel containing cells; the second substrate further includes: a second hydrogel channel configured to be filled, at least partially, with the hydrogel containing cells, wherein an end of the second hydrogel channel is vertically-aligned with the second loading port; a third through-hole that is vertically-aligned with the second inlet; and a fourth through-hole that is vertically-aligned with the second outlet; and the third substrate further includes: a second media channel configured to receive the fluid media, wherein a first end of the second media channel is vertically-aligned with the third through-hole and a second end of the second media channel is vertically-aligned with the fourth through-hole.
123. The microfluidic chip of embodiment 122, wherein: the inlet and the second inlet are spaced along a first side edge of the first substrate; and the outlet and the second outlet are positioned at a second side edge of the first substrate, the second side edge opposite the first side edge.
124. A device for use in a bone marrow model, including: a top substrate including: an inlet defined in a top of the top substrate, the inlet configured to allow fluid media to ingress into the device; an outlet defined in the top of the top substrate, the outlet configured to allow the fluid media to egress from the device; and a loading port defined in the top of the top substrate, the loading port configured to receive a hydrogel containing cells; a bottom substrate including a media channel configured to receive the fluid media; an intermediate substrate interposed between the top substrate and the bottom substrate, the intermediate substrate including: a hydrogel channel configured to be filled, at least partially, with the hydrogel containing cells, wherein an end of the hydrogel channel is vertically-aligned with the loading port; a first through-hole that is vertically-aligned with the inlet and with a first end of the media channel; and a second through-hole that is vertically-aligned with the outlet and with a second end of the media channel; and a permeable barrier interposed between the intermediate substrate and the bottom substrate.
125. The device of embodiment 124, wherein the media channel spans a center of the bottom substrate and is oriented horizontally on the bottom substrate.
126. The device of embodiment 124, wherein the hydrogel channel spans a center of the intermediate substrate and is oriented horizontally on the intermediate substrate.
127. The device of embodiment 124, wherein: a center portion of the hydrogel channel is straight; and a peripheral portion of the hydrogel channel is curved.
128. The device of embodiment 124, wherein: the top substrate further includes a second loading port defined in the top of the top substrate; and the hydrogel channel includes a second end that is vertically-aligned with the second loading port.
129. The device of embodiment 124, wherein: the top substrate further includes: a second inlet defined in the top of the top substrate, the second inlet configured to allow the fluid media to ingress into the device; a second outlet defined in the top of the top substrate, the second outlet configured to allow the fluid media to egress from the device; and a second loading port defined in the top of the top substrate, the second loading port configured to receive the hydrogel containing cells; the bottom substrate further includes a second media channel configured to receive the fluid media; and the intermediate substrate further includes: a second hydrogel channel configured to be filled, at least partially, with the hydrogel containing cells, wherein an end of the second hydrogel channel is vertically-aligned with the second loading port; a third through-hole that is vertically-aligned with the second inlet and with a first end of the second media channel; and a fourth through-hole that is vertically-aligned with the second outlet and with a second end of the second media channel.
130. The device of embodiment 129, wherein: the inlet and the second inlet are spaced along a first side edge of the top substrate; and the outlet and the second outlet are positioned at a second side edge of the top substrate, the second side edge opposite the first side edge.

### Example 1: Representative Method to Prepare an Endosteal Niche

This example provides a protocol for preparing an endosteal niche for use in the designs and methods herein.

Osteopontin (OPN) stock: 5 mg of OPN was dissolved in 1 mL of DMEM-HEPES basal medium (without fetal bovine serum (FBS) and penicillin and streptomycin (P/S)). The mixture was ultrasonicated for 15 min (or until completely dissolved), then aliquoted into small vials and stored at −20° C. The working stock was sonicated for 10 min prior to adding it to mineralization solution.

DMEM-HEPES complete medium: DMEM-HEPES basal medium (Gibco 12320-032) supplemented with 10% FBS+50 to 100 I.U./mL penicillin and 50 to 100 (μg/mL) streptomycin.

Solution-1: Calcium chloride solution (9 mM): 13.23 mg CaCl₂.2H₂O was dissolved in 10 mL of DMEM-HEPES complete medium and filtered using 0.2 μm syringe filter. Stored at 4° C. for up to 2 weeks.

Solution-2: Potassium Phosphate solution (4.2 mM): 7.31 K₂HPO₄ was dissolved in 10 mL of DMEM-HEPES complete medium and filtered using 0.2 μm syringe filter. Stored at 4° C. for up to 2 weeks.

Mineralizing Medium: This medium was prepared fresh (from stocks) before use for every media change. An OPN stock aliquot was thawed and ultrasonicated for 10 min., then Solution-1 was mixed thoroughly with the OPN stock to get a final concentration of 100 μg/mL. Solution-2 was added to this mixture. The order of mixing components is important. For preparing 10 mL Mineralizing medium: 5 mL of Solution-1 was mixed with 200 μl of OPN stock (5 mg/mL) and then 5 mL of Solution-2 was added to this.

Collagen Gel: Collagen gel was prepared on ice; it should not be allowed to reach room temperature. A 2 mg/mL Collagen Gel was prepared using 66.67 μL of 3 mg/mL Type I collagen, 10 μL of 10×PBS, and 1.7 μL of 1N NaOH.

Cells (5×10⁴) were suspended in 21.674 μL DMEM-HEPES complete medium; cells were suspended in 38.75 μL medium), then added to the prepared collagen gel to get a final 100 μL gel volume. [Note: Cell number greater than 5×10⁵ cells/mL results in poor gelation and faster gel degradation.] This was mixed thoroughly without forming bubbles.

The drop was added onto the well plate and allowed to crosslink for 1 hr in a humidified incubator at 37° C. Once cross-linked, the transparent gel turned opaque. The freshly prepared mineralizing medium was then added along the walls of the well, and the plate placed in a rotary shaker inside the humidified 37° C. incubator. The old medium was replaced with fresh mineralizing medium every 24 hrs for 3 days to achieve mineralization. Mineralizing medium is prepared fresh every time, immediately before addition. Once the gel was mineralized after 3 days, the mineralizing medium was replaced with DMEM-HEPES complete medium for the rest of the study period.

As characterized previously (Thrivikraman et al., Nature Comm., 10(1):3250, 2019; WO 2020/069033), this process provides a mineralized hydrogel material having an ultrastructure similar to native bone; this mineralized hydrogel material is useful in the designs and methods provided herein. It is chemically similar to native bone in FTIR spectral, mineral to matrix ratio, and crystallinity index aspects (Thrivikraman et al., Nature Comm., 10(1):3250, 2019; WO 2020/069033). Mineralized samples had comparable values to that of native bone, and both were significantly higher than non-mineralized controls (****p<0.0001 ANOVA/Tukey). Mineral crystallinity was calculated from the FT-IR spectra suggestive of native bone-like apatite crystallinity in mineralized microenvironment. Crystallinity index was calculated from the extent of splitting of the two absorption bands at 605 and 565 cm-1.

Elements of the endosteal niche (such as the mineralized collagen) mentioned above can be cut, broken, or mechanically agitated to form “shards”. Shards may or may not encapsulate cells. Shards can be associated with the bone marrow model in a variety of ways, for instance they may be embedded throughout the cell laden hydrogel (for instance, in the vascular niche).

### Example 2: Formation of Endosteal Niche Shards

Shards were created by mechanical dissociation of mineralized collagen/cell endosteal niche. These shards enable elements of the niche to be applied throughout stem cell laden matrix. They may be created to varying sizes depending upon channel width or other needs.

After formation of the mineralized niche such as described in Example 1, mineralized disks were removed from their wells and placed into a 35 mm tissue culture dish containing enough DMEM-HEPES complete media to embed but not cover the disks (about 3-5 ml). Using a clean rounded scalpel, the disks were then chopped into roughly 0.5-1 mm² pieces. The media and mineralized pieces were then passed through gradually smaller needles (18 g, 23 g, 25 g) with a 5 ml syringe to create the final shard morphology. Shards were pipetted into a microcentrifuge tube and spun at 300×g for 3 min. Used media was removed and replaced with fresh DMEM+ HEPES complete. Shards+media were transferred to a tissue culture well and placed into a 37° C. incubator with humidified atmosphere containing 5% CO₂.

Optionally, these shards are seeded with live cells, for instance human fetal osteoblasts (hfOB), as described herein.

### Example 3: Formation of Mineralized Collagen Drops Seeded with Human Fetal Osteoblasts (hfOBs)

This example provides a representative method for formatting mineralized hydrogel (exemplified using rate tail collagen 1) drops/beads/spheres, and loading them with live cells (exemplified with human fetal osteoblasts).

A cell suspension of hfOBs was prepared in Osteoblast growth medium (HObGM; Cell Applications Inc. #417-500) at a concentration of 1×10⁶ cells/mL.

Several drops of silicon oil were added into each well of a U-bottom 96-well plate.

Collagen mixture was prepared essentially as above, by mixing 3.4 μL 1N NaOH with 16.7 μL 10×DPBS in a 1.5 mL Eppendorf tube, to make a 3.76 mg/mL stock. 147 μL of Rat tail collagen 1 was carefully pipetted into the tube without introducing bubbles, the pipetted up and down to mix thoroughly. 100 μL cell suspension or 100 μL of cell-free media was added to the freshly collagen mixture, pipetting up and down to mix. Using a p2 pipettor, a 0.2 μL aliquot of the collagen mixture was placed into the bottom of each well, then incubated at 37° C. for 45 minutes.

A 15 mL conical tube was prepared with 3-4 mL OGM media, then the collagen drops into this using a wide-bore pipette tip, and allowed to settle to bottom of media layer. The upper oil layer was removed and disposed of, then the media was siphoned off, leaving ˜1 mL.

A wide bore pipette tip was used to transfer solution bearing collagen drops into wells of a 24-well dish (˜4 drops per well, to minimize aggregation). Each well was then filled with mineralizing solution as described in Example 1 but with HObGM media in place of the DMEM-HEPES basal medium.

The mineralizing solution was carefully exchanged without siphoning up collagen drops each day for a total of 3 rounds of freshly made mineralizing solution. Following 3 days of mineralization, media bearing mineralized drops was transferred from all wells into a 15 mL conical tube and allowed to settle. The majority of media was removed, the beads washed with 10 mL DMEM+FBS+S/P, then transferred to a well where they are cultured in DMEM+FBS+S/P until needed.

### Example 4: Vascular Niche Protocol

This example describes a representative vascular niche experiment including hMSCs and human primary Bone Marrow CD34+ Cells (HSCs) seeded into MATRIGEL® basement membrane.

hMSC Cells were used from passages 2-6. Prior to experiments, MSCs were cultured in Mesenchymal Stem Cell Basal Medium for Adipose, Umbilical and Bone Marrow-derived MSCs (ATCC PCS-500-030) supplemented with Mesenchymal Stem Cell Growth Kit for Bone Marrow-derived MSCs (ATCC PCS-500-041) containing rh FGF basic: 125 pg/mL, rh IGF-1: 15 ng/mL, Fetal Bovine Serum: 7%, L-Alanyl-L-Glutamine: 2.4 mM and 1% antibiotic solution. Cells were dissociated using TrypLE express (Gibco) and centrifuged at 300×g for 3 minutes. Cells were then suspended in growth media at desired concentrations. HSCs were stored in liquid N₂ until the day of the experiment, then thawed, suspended in Hanks Balanced Salt Solution at 1:10 ratio, and spun at 300×g for 3 minutes. Media was carefully drawn off and the cell pellet suspended in the appropriate amount of MSC growth media.

Prior to cell dissociation (hMSC) and thawing (HSC), MATRIGEL® was allowed thaw. Cells were mixed with MATRIGEL® in a 1:1 cell containing solution to MATRIGEL® mixture. At this point the cell, MATRIGEL® mixture optionally may be seeded with human osteoblast-containing mineralized shards, or mineralized collagen spheres. Further an entire disk of mineralized collagen containing human osteoblasts may be laid into a channel of the microfluidic device prior to this point.

The seeded MATRIGEL® mixture was pipetted into the designated channel of the microfluidic device, and the seeded microfluidic device was placed into a humid 37° C. incubator with a 5% CO₂ atmosphere. The cell seeded MATRIGEL® was allowed to gel for a minimum of 30 minutes, after which the device was removed and the appropriate growth media (here, SFEM II medium (STEMCELL Technologies, 09655) supplemented with 10% FBS, 100 U/l penicillin and 100 mg/ml streptomycin, 12.5 μg/ml aprotinin, 20 ng/ml EPO, 1 ng/ml G-CSF, 100 ng/ml Flt3-L, 100 ng/ml TPO, 50 ng/ml SCF, and select EGM-2 BULLETKIT™ (Lonza, CC-4176) components (hFGF-B, VEGF, R3-IGF-1, hEGF, ascorbic acid, and heparin) according to the manufacturer's instructions) was fed through adjoining channels.

Alternative cytokine mixtures have also been tested, including media which contains 20 ng/ml EPO and 100 ng/ml TPO but no aprotinin, G-CSF, SCF or EGM-2 Bullet kit reagent and others which contain half the previous amounts of EPO and TPO but are otherwise cytokine free, see Table 3 in Example 11. The entire device, media and pumping apparatus was placed into the incubator for the entire length of the experiment. The microfluid devices were periodically removed for inspection of channel(s) containing cells and/or media. Media may be replaced depending upon the condition of the experiment to solution containing growth factors, drugs or other.

### Example 5: Ring Protocol

Mineralized cell-seeded collagen rings were created by removing an inner area of the endosteal niche disk described herein by use of a 1 mm-3 mm biopsy punch. Cell-seeded hydrogel ‘plugs’ were then added to the center void and allowed to cross link at 37° C. in a humid atmosphere containing 5% CO₂ for at least 30 minutes and up to 1 hour. Appropriate growth media was then added, depending upon experimental conditions.

FIG. 37 presents a ring design of this embodiment, showing a mineralized outer layer encapsulating osteoblasts, and a non-mineralized core encapsulating mesenchymal stem cells.

### Example 6: Disk Protocol

Disks were created by using applying approximately 100 μl osteoblast-seeded collagen onto the center of a well within a 24-well tissue culture plate. Sizes may be scaled up or down by changing well size and volume used. This mixture was allowed to cross link at 37° C. in a humid atmosphere containing 5% CO₂ for at least 30 minutes and up to 1 hour. One milliliter of warmed mineralization media was added to the well and the plate was placed upon a 2-d shaker inside a 37° C. incubator with humid atmosphere and 5% CO₂. Media was removed daily and fresh media was added for three days, after which DMEM containing, 1 g/L sucrose, 25 mM HEPES, 10% FBS, and 1% antibiotics was used. Shrinkage of the disks was normal and may be up to 50% of original size after mineralization.

### Example 6: Microvascular Capillaries

Other embodiments in the designs and methods herein include pericyte-supported vascular endothelial capillaries, vascularizing the endosteal niche. Endothelial cells can be added before or after mineralization; they then self-assemble into a vascular capillaries by themselves in a process of vascular morphogenesis. This example provides a representative protocol; see also Thrivikraman et al., Nature Comm., 10(1):3250, 2019; WO 2020/069033.

BM-MSC cultured and human umbilical vein endothelial cells (HUVECs) were encapsulated in collagen (as described above) at a ratio of 4:1 to a final concentration of 2.5×10⁶ cells/mL, the cultured for 3 days in a 4:1 endothelial growth medium (EGM) EGM-DMEM medium, supplemented with 2 ng/ml of TGF-β.

Next, the collagen constructs were mineralized using the standard DMEM mineralization medium described herein (with OPN, Ca and P) supplemented with EGM™ BULLETKIT™ growth factors, without TGF-β.

### Example 7: Niche Separator

Embodiments of the provided bone marrow models may optionally include a niche separator. This example describes representative niche separators and how to make and use them.

Resin niche separators were designed using CAD (Fusion 360, Autodesk) for both positive and negative versions. Positive resin niche separators were printed (Ember, Autodesk) and cleaned with ethanol following manufacturer recommendations. The printed samples underwent further post processing steps of curing lights for 20 minutes (Triad II, Dentsply) and overnight storage at 80° C. to reduce the amount of unreacted monomer present. These parts were used as impression templates in which Polydimethylsiloxane (Sylgard 184, Dow Corning) was cast around the template. After solidification, the printed resin part was removed from the impression material. Fluidic connection ports were biopsy punched into the device and fluidic connectors are attached during device use.

Negative niche separators were 3D printed using the same resin as the negative forms. These versions were designed to be the final form of the micro fluidic device, not requiring additional molding steps. The cleaning process for these resin devices was the same as the positive devices and followed the previously described cleaning and post processing steps. PDMS molds were attached to glass coverslips via plasma treatment (Plasma Cleaner PDC-32G, Harrick Plasma). Resin devices were attached to glass using super glue adhesive.

### Example 8: BDMS HERO™ Sandwich Chip

Examples of the provided bone marrow models are in the form of sandwich (HERO™) chips. This example describes one HERO sandwich chip that includes two channels that may or may not be separated by a biocompatible permeable barrier.

Protocol for fabrication of polydimethylsiloxane (PDMS) HERO Chips: The PDMS Hero chips were composed of three parts (from top to bottom): Upper channel: PDMS at least 3 mm thick with a 0.5 mm thick channel; PETE porous membrane (porosity of 10 μm); and Bottom channel: PDMS 1 mm thick with 0.4 mm thick channel.

PDMS molds were fabricated with the desired channel shape and dimensions. This can be done using different known methods, such as computerized numerical control (CNC), laser cutting, and so forth. PDMS (ratio 1:10 curing agent:base elastomer) was prepared and poured on the aforementioned molds, the filled molds were degassed and cured overnight at room temperature. Alternatively, the degassed filled molds were cured at 80° C. for 2 hours. Inlet and outlet holes were made using a puncher.

The PDMS chips were attached with PET membrane, as follows: 5% (3-aminopropyl)triethoxysilane (APTES) was prepared in deionized water. The PDMS was rinsed with isopropyl alcohol (IPA), then dried with nitrogen; immersed in IPA and sonicated for 20 minutes, and again dried with nitrogen, then placed at 80° C. for at least 20 min.

The PET membrane was prepared as follows: the membrane was inserted into the appropriate support, and plasma cleaned/etched using O₂ plasma (35 W, 45 s). The cleaned membrane was moved to a new support, and a thin layer of the APTES dilution created over the membrane. It was then placed at 70° C. for 30 min.

The PDMS and Membrane was placed on a new support, then the PDMS was treated with O₂ plasma (35 W, 45 s) and the APTES-treated membrane and PDMS treated with O₂ plasma of (35 W, 15 s). The surfaces were then brought together, and mechanical pressure applied for 1 hour. The procedure was repeated for the next layer.

The chip was flowed and rinsed with IPA, then sterilized with 70% ethanol followed by UV for 30 min. The prepared and sterile chip was treated overnight with fibronectin (100 μg/ml in dPBS) by pipetting solution into channels, removing excess from chip surface, and incubating at 37° C. The following day, channels were washed with and re-filled with dPBS. The chips were then stored at 4° C. in a humid environment.

### Example 9: Acrylic HERO™ Chip

Another embodiment is an Acrylic HERO chip. This example provides a description of how to make a representative acrylic HERO sandwich chip. By way of example, such a chip includes five layers (from top to bottom): Upper cover: Made of Acrylic of 3 mm thick; Culture Media channel layer: Made of Acrylic of 0.384 mm; PETE porous membrane (porosity of 10 μm); MATRIGEL® layer: Made of Acrylic of 0.178 mm; and Bottom cover: Made of high precision acrylic of 0.5 mm thick. As illustrated in FIG. 47, an example acrylic HERO™ sandwich chip includes acrylic cover (4701), a culture media channel layer, represented by layer material or sheet 4702 and channel 4703, permeable barrier or membrane (4704), a vascular and/or endosteal channel layer, represented by layer material or sheet 4705 and channel 4706, and glass slide 4707.

Assembly of an Acrylic Hero Chip: Each of the aforementioned acrylic layers was fabricated using CNC or Laser cutting techniques. Doubles side tape was used to assemble the upper and the bottom parts of the chip, then each part was rinsed with IPA. The assembly was sterilized with 70% ethanol, then treated with UV light for 30 min. The bottom part of the chip was plasma treated similarly to described in Example 7, and the chip assembled. It as then treated overnight with fibronectin.

### Example 10: In Vivo Model

This example describes a method of making an in vivo bone marrow model in a disk format, using bmMSCs.

Disks and shards were created as described above. A volume of shards equal to two 100 μl disks was suspended in media containing 2.0×10⁶ cells/ml bmMSCs. An equal volume of MATRIGEL® was diluted with this mixture. The hydrogel mixture was injected subcutaneously onto the haunches of NGS mice.

For the disks experiment, two whole disks were suspended in a volume of bmMSC equal to that of the shards, this was mixed with MATRIGEL® and injected into mice as described above.

Control gels: cells were suspended in MATRIGEL® but without addition of shards or disks.

The mice were observed for 8 weeks. At eight weeks, the nodules at injection sites were removed, stained with H&E and inspected for infiltration of hematopoietic cells into them.

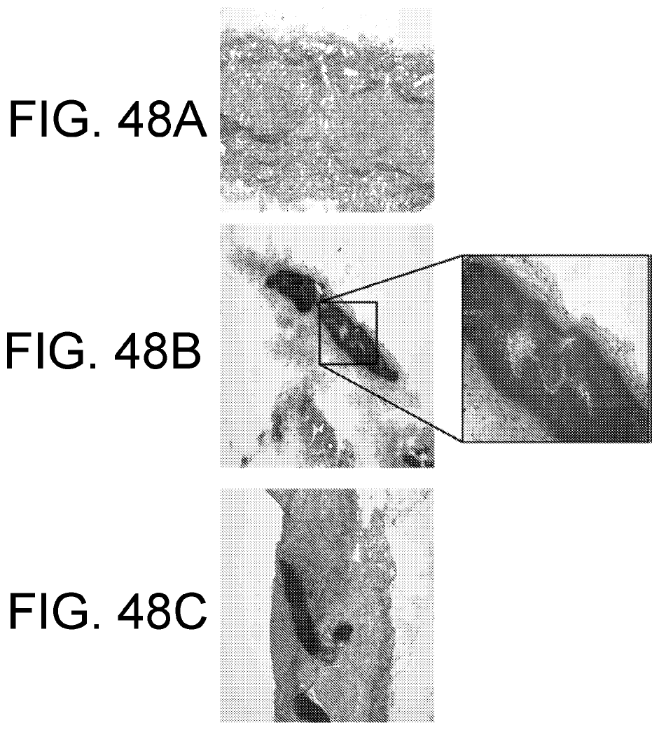

Results: Mice containing shards or disks developed hardened nodules at injection sites. whereas approximately only one of four injection sites of gel control evolved this nodule. H&E staining showed minimal infiltration at all sites. FIG. 48A shows endosteal free gel; FIG. 48B (plus inset) shows gel containing a mineralized disk. FIG. 48C shows gel containing mineralized shards.

Experiment 11: Alternative Media Containing Different Cytokine Loads

This example describes preparation and testing of bone marrow model microenvironment niches using different media and cytokine loads.

Shards were created as described above.

A fibrin hydrogel was created containing 0.2 mg/ml Collagen Type I, 25 μg/ml Aprotinin, 0.5 U/ml Thrombin and 5 mg/ml fibrinogen, bone marrow derived MSCs, CD34+ HSCs and shards. A 100 μl volume contained 2.0×10⁴ mesenchymal stem cells, 1.0×10⁴ CD34+ hematopoietic stem cells and shards from a 50 μl disk before mineralization.

25 ul of this mixture was applied to the bottom of wells of a tissue culture treated U- or V-bottomed 96-well microplate. The hydrogel mixture was allowed to cross link inside a 37° C. tissue culture incubator for 60 minutes.

Pre-warmed culture full media (as in Example 3) was applied gently to each well. The culture media contained: SFEM II (Stemcell technologies cat: 09655), 10% fetal bovine serum, 100 I.U./mL penicillin and 100 (μg/mL) streptomycin, 12.5 μg/ml Aprotinin, 20 ng/ml EPO, 1 ng/ml G-CSF, 100 ng/ml Flt3-L, 100 ng/ml TPO, 50 ng/ml SCF, and select EGM-2 BulletKit (Lonza, CC-4176) components (hFGF-B, VEGF, R3-IGF-1, hEGF, ascorbic acid, and heparin) according to the manufacturer's instructions. Plates were placed back into incubators and allowed to culture for 7 days. 50% media changes were performed every other day.

On day 8 media of the “Low” cytokine cohort was replaced with: SFEM II (Stemcell technologies cat: 09655), 10% fetal bovine serum, 100 I.U./mL penicillin and 100 (μg/mL) streptomycin, 20 ng/ml EPO, and 100 ng/ml TPO.

On day 8 media of the “Half” cytokine cohorts was changed to: SFEM II (Stemcell technologies cat: 09655), 10% fetal bovine serum, 100 I.U./mL penicillin and 100 (μg/mL) streptomycin, 10 ng/ml EPO, and 50 ng/ml TPO.

Media changes were performed 3× per week for all cohorts.

At two and four weeks, hydrogels were digested using 1 mg/ml collagenase, 2.5 mg/ml Nattokinase in a 25 mM HEPES/DMEM buffer for approximately 1 hour. Samples were spun at 300×g and the resultant pellet suspended in flow cytometry staining buffer, stained for appropriate surface markers and interrogated on a flow cytometer. Table 3 shows mean numbers of total cells per sample, CD45+ cells, and CD45+ CD34+ cells.

Results: Table 3 shows some loss of per sample size from two weeks to four weeks. It also shows not significant change in the number and frequency of CD45+ and CD45+ CD34+ cells per cytokine load. Based on this, it is believed that the MSCs present in the sample provide necessary cytokines for hematopoietic cell culture.

TABLE 3
Average counts and frequencies for cytokine experiment

| | | Total Single | | | | |
|---|---|---|---|---|---|---|
| | | Live Cells | CD45+ Cells | | CD45+ CD34+ Cells | |
| Timeline | Media | Count | Count | Frequency | Count | Frequency |
| Two Week | Full | 14815.5 | 11886.125 | 80.23% | 354.125 | 2.98% |
| | Half | 14461.75 | 10297.875 | 71.21% | 157.25 | 1.53% |
| | Low | 12525 | 8742.25 | 69.80% | 113.375 | 1.30% |
| Four Week | Full | 8376 | 2577.75 | 30.78% | 58 | 2.25% |
| | Half | 11610.5 | 3176 | 27.35% | 42 | 1.32% |
| | Low | 9539.5 | 3708.375 | 38.87% | 65.875 | 1.78% |

Counts represent means. CD45+ Cell frequency represents frequency of total live single cells. CD45+ CD34+ Cell frequency represents frequency of total live single cells.

Experiment 12: Use of Endothelial Monolayer Separating Media and Vascular Niche, and Shards Versus No Shards

This example describes a multiparameter experiment testing different embodiments of bone marrow models.

Shards were created as previously described.

For “Shard” experiments in commercially available chips (exemplified with S-1 microfluidic chips from Emulate, Inc.) and in 96-well TC plates (Static embodiment), fibrin hydrogels were created containing 0.2 mg/ml Collagen Type I, 25 ug/ml Aprotinin, 0.5 U/ml Thrombin and 5 mg/ml fibrinogen, bone marrow derived MSCs, CD34+ HSCs and shards. A 100 μl volume contained 2.0×10⁴ mesenchymal stem cells (MSCs), 1.0×10⁴ CD34+ hematopoietic stem cells, and shards from a 50 μl disk before mineralization.

For “No Shard” experiments, fibrin hydrogels were created containing 0.2 mg/ml Collagen Type I, 25 μg/ml Aprotinin, 0.5 U/ml Thrombin and 5 mg/ml fibrinogen, bone marrow derived MSCs and CD34+ HSCs. A 100 μl volume contained 2.0×10⁴ mesenchymal stem cells, 1.0×10⁴ CD34+ hematopoietic stem cells.

For static samples, 25 μl of the appropriate mixture was applied to the bottom of wells of a tissue culture treated U- or V-bottomed 96-well microplate.

Emulate-type experiments contain a HUVEC monolayer created by adding a 4.0×10⁶ cells/ml HUVEC suspension into the media channel of a microfluidic chip, inverting the chip and incubating for 2 hours at 37° C. The cell suspension was then gently washed out and replaced with endothelial growth media (EGM™-2 Endothelial Cell Growth Medium-2 BulletKit, LONZA CC-3162). Cells were allowed to culture for 24 hours prior to the addition cell-laden hydrogels into the upper, cell channel.

For Emulate samples, 25 μl of the appropriate mixture was applied to the upper (cell) channel an Emulate S-1 microfluidic chip. The hydrogel mixture was allowed to cross link inside a 37° C. tissue culture incubator for 60 minutes.

Pre-warmed culture media was applied gently to each sample. Emulate chips were run at 30 μl/hr. Static wells contained 200 μl of media per well changes three times per week. The following culture media was used: SFEM II (Stemcell technologies cat: 09655), 10% fetal bovine serum, 100 I.U./mL penicillin and 100 (μg/mL) streptomycin, 12.5 μg/ml Aprotinin, 20 ng/ml EPO, 1 ng/ml G-CSF, 100 ng/ml Flt3-L, 100 ng/ml TPO, 50 ng/ml SCF, and select EGM-2 BulletKit (Lonza, CC-4176) components (hFGF-B, VEGF, R3-IGF-1, hEGF, ascorbic acid, and heparin) according to the manufacturer's instructions.

Samples were placed back into incubators and allowed to culture for 14 days.

At two weeks, all hydrogels in chips and in wells were digested using 1 mg/ml Collagenase, 2.5 mg/ml Nattokinase in a 25 mM HEPES/DMEM buffer for approximately 1 hour. Samples were spun at 300×g and suspended in flow cytometry staining buffer, stained for appropriate surface markers and interrogated on a flow cytometer. Table 4 shows mean numbers of total cells per sample, CD45+ cells and CD45+ CD34+ cells.

Results: Table 4 shows similar frequency of CD45+ and CD45+CD34+ cells across conditions. The static cohort of this experiment had a slightly higher frequency of both marker groups. The Shard, No HUVEC cohorts have lower overall cell count but CD45+ and D45+CD34+ frequencies are similar. Based on this, it is believed that the addition of an endothelial monolayer does not lead to significant disruption of the vascular niche when compared to fluidic samples lacking this and compared with static samples.

TABLE 4
Average counts and frequencies for multiparameter experiment

| | | | Total | | | | |
|---|---|---|---|---|---|---|---|
| | | | Single | | | | |
| | | | Live | | | CD45+ | |
| | | | Cells | CD45+ Cells | | CD34+ Cells | |
| Type | Shard | HUVEC | Count | Count | Frequency | Count | Frequency |
| Emulate | Shard | HUVEC | 50764.3 | 41248 | 81.25% | 1566 | 3.80% |
| | | No HUVEC | 21728.3 | 17019.83 | 78.33% | 925 | 5.43% |
| | No Shard | HUVEC | 68478.6 | 49927.8 | 72.91% | 2182.4 | 4.37% |
| | | No HUVEC | 53403.5 | 42023.67 | 78.69% | 2284.67 | 5.44% |
| | | | Count | Count | Frequency | Count | Frequency |
| Static | Shard | No HUVEC | 41506.7 | 34668.67 | 83.53% | 2554.67 | 7.37% |
| | No Shard | No HUVEC | 29225.5 | 23940.5 | 81.92% | 1619.5 | 6.76% |

Counts represent means.
CD45+ Cells frequency represents frequency of Total Single Live Cells.
CD45+ CD34+ Cells frequency represents frequency of CD45+ Cells.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is any alteration in a method, composition, model, or procedure that would significantly alter the ability of a model to represent biological activity(s) of a bone tissue or microenvironment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed.

Anthony Smith, Oxford University Press, Oxford, 2004).

1. A bone marrow model comprising:
a first microenvironment comprising a hydrogel with or without cells; and
a second microenvironment comprising:
a hydrogel with cells found in a normal hematopoietic niche of normal bone marrow; or
a hydrogel with cells found in a condition selected from the group consisting of hematologic malignancies, marrow failure syndromes, pre-malignant clonal hematopoiesis, and solid tumor metastases to bone marrow.

2. The bone marrow model of claim 1, wherein the first microenvironment comprises a mineralized hydrogel.

3. A bone marrow model comprising:
a first microenvironment comprising a hydrogel with mature bone cells; and
a second microenvironment comprising a hydrogel with cells found in a hematopoietic niche of normal bone;
wherein the first microenvironment and second microenvironment are maintained in a position where they can interact with a fluid medium.

4. The bone marrow model of claim 3, wherein the first microenvironment comprises a mineralized hydrogel.

5. A bone marrow model comprising:
a first microenvironment comprising a hydrogel with immature bone cells; and
a second microenvironment comprising a hydrogel with cells found in a hematopoietic niche of normal bone;
wherein the first microenvironment and second microenvironment are maintained in a position where they can interact with a fluid medium.

6. The bone marrow model of claim 5, wherein the first microenvironment comprises a mineralized hydrogel.

7. A bone marrow model comprising:
a first microenvironment comprising a hydrogel without cells or with mature bone cells and/or mesenchymal stem cells; and
a second microenvironment comprising a non-mineralized hydrogel incorporating elements of a hematopoietic niche;
wherein the first microenvironment is in contact with the second microenvironment.

8. The bone marrow model of claim 7, wherein the first microenvironment comprises a mineralized hydrogel.

9. A bone marrow model comprising:
a first microenvironment comprising a hydrogel with mature bone cells; and
a second microenvironment comprising:
a hydrogel with cells found in a normal hematopoietic niche of normal bone marrow; or
a hydrogel with cells associated with a diseased state in a hemopoietic niche.

10. The bone marrow model of claim 9, wherein the first microenvironment comprising a mineralized hydrogel.

11. A bone marrow model comprising:
a first microenvironment comprising a hydrogel with immature bone cells and/or mesenchymal stem cells; and
a second microenvironment comprising:
a hydrogel with cells found in a normal hematopoietic niche of normal bone marrow; or
a hydrogel with cells associated with a diseased state in a hemopoietic niche.

12. The bone marrow model of claim 11, wherein the first microenvironment comprising a mineralized hydrogel.

13. The bone marrow model of claim 9, wherein the second microenvironment comprises a hydrogel with cells found in a condition selected from the group consisting of hematologic malignancies, marrow failure syndromes, pre-malignant clonal hematopoiesis, and solid tumor metastases to bone marrow.

14. A bone marrow model comprising:
a first microenvironment comprising a mineralized hydrogel without cells or with mature bone cells and/or mesenchymal stem cells; and
a second microenvironment comprising a non-mineralized hydrogel incorporating elements of a hematopoietic niche;
wherein the first microenvironment is in contact with the second microenvironment.

15. A bone marrow model comprising:
a first microenvironment comprising a mineralized hydrogel without cells or with bone cells or mesenchymal stem cells; and is implemented as mineralized fragments in the form of shards; and
a second microenvironment comprising a hydrogel with cells found in the hematopoietic niche of normal, modified, or diseased bone marrow;
wherein the mineralized hydrogel of the first microenvironment is embedded in the hydrogel of the second microenvironment and the hydrogels of both the first and second microenvironments are maintained in a position where they can interact with a fluid medium.

16. A bone marrow model comprising:
a) a first microenvironment comprising a mineralized hydrogel without cells or with mature bone cells or mesenchymal stem cells; and
b) a second microenvironment comprising a hydrogel material with cells found in the hematopoietic niche of normal, modified, or diseased bone marrow;
wherein the layers of hydrogel referenced above a) is embedded in b) and a) and b) are maintained in a position where they can interact with a fluid medium.

17. The bone marrow model of claim 16, wherein the mineralized hydrogel is in a form spheres, spheroids, beads, and/or droplets.

18. The bone marrow model of claim 2, wherein the mineralized hydrogel of the first microenvironment is prepared by a method comprising:
combining the living cells with a fluid medium and unlinked polymer chains to create a first composition;
treating the first composition with a cross-linking agent or cross-linking treatment sufficient to cross-link the polymer chains to form a cross-linked polymer matrix comprising the living cells; and
treating the cross-linked polymer matrix comprising the living cells with at least one mineralizing solution for a time sufficient to form the composition of a mineralized cross-linked polymer matrix comprising the living cells.

19. The bone marrow model of claim 2, wherein the mineralized hydrogel of the first microenvironment is prepared by a method comprising:
combining unlinked polymer chains with a fluid medium to create a first composition;
treating the first composition with an agent or treatment sufficient to cross-link or entangle the polymer chains to form a polymer matrix;
combining the living cells with the cross-linked polymer matrix to form a polymer matrix comprising the living cells; and
treating the polymer matrix comprising the living cells with at least one mineralizing solution for a time sufficient to form the composition of a mineralized polymer matrix comprising the living cells.

20. The bone marrow model of claim 2, wherein the mineralized hydrogel of the first microenvironment is prepared by a method comprising:
combining the living cells with a fluid medium and acid solubilized Type 1 collagen chains to create a first composition;
maintaining the first composition under conditions sufficient to cause the Type 1 collagen chains of the first composition to undergo fibrillogenesis and form a second composition comprising the living cells; and
treating the second composition comprising the living cells with at least one mineralizing solution for a time sufficient to form the composition of a mineralized Type 1 collagen matrix comprising the living cells.

21. The bone marrow model of claim 2, wherein the mineralized hydrogel of the first microenvironment is prepared by a method comprising:
combining a fluid medium and acid solubilized Type 1 collagen chains to create a first composition;
maintaining the first composition under conditions sufficient to cause the Type 1 collagen chains of the first composition to undergo fibrillogenesis and form a second composition;
combining the living cells with the second composition to form a third composition comprising the living cells; and
treating the third composition comprising the living cells with at least one mineralizing solution for a time sufficient to form the composition of a mineralized Type 1 collagen matrix comprising the living cells.

22. The bone marrow model of claim 2, wherein the mineralized hydrogel of the first microenvironment is prepared by a method comprising:
combining the living cells with a fluid medium and acid solubilized Type 1 collagen chains to create a first composition;
adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 until the Type 1 collagen chains undergo fibrillogenesis and form a second composition comprising the living cells; and
treating the second composition comprising the living cells with at least one mineralizing solution for a time sufficient to form the composition of a mineralized Type 1 collagen matrix comprising the living cells.

23. The bone marrow model of claim 2 wherein the mineralized hydrogel of the first microenvironment is prepared by a method comprising:
combining a fluid medium and acid solubilized Type 1 collagen chains to create a first composition;
adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 until the Type 1 collagen chains undergo fibrillogenesis and form a second composition;
combining the living cells with the second composition to form a third composition comprising the living cells; and
treating the third composition comprising the living cells with at least one mineralizing solution for a time sufficient to form the composition of a mineralized Type 1 collagen matrix comprising the living cells.

24. The bone marrow model of claim 2, wherein the mineralized hydrogel of the first microenvironment is prepared by a method comprising:
combining the living cells with a fluid medium and acid solubilized Type 1 collagen chains to create a first composition;
adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 and a temperature of from about 34° C. to about 40° C. until the Type 1 collagen chains undergo fibrillogenesis and form a second composition comprising the living cells; and
treating the second composition comprising the living cells with at least one mineralizing solution for a time sufficient to form the composition of a mineralized Type 1 collagen matrix comprising the living cells.

25. The bone marrow model of claim 2, wherein the mineralized hydrogel of the first microenvironment is prepared by a method comprising:
combining a fluid medium and acid solubilized Type 1 collagen chains to create a first composition;
adjusting the pH of the first composition to 6-8, or from 6.5-8, or from 6.8-7.8, or from 7.0-7.8, or from 7.2-7.8, or from 7.2-7.6, or to about 7.6 and maintaining the first composition at a pH of 6-8, or from 6.5-8, or from 6.8-7.8, or from 7.0-7.8, or from 7.2-7.8, or from 7.2-7.6, or about 7.6 and a temperature of from about 34° C. to about 40° C. until the Type 1 collagen chains undergo fibrillogenesis and form a second composition;
combining the living cells with the second composition to form a third composition comprising the living cells; and
treating the third composition comprising the living cells with at least one mineralizing solution for a time sufficient to form the composition of a mineralized Type 1 collagen matrix comprising the living cells.

26. The bone marrow model of claim 17, wherein the mineralizing solution comprises:
from about 3.0 mM to about 6.0 mM of calcium ions; or
from about 4.0 mM to about 5.0 mM of calcium ions.

27. The bone marrow model of claim 17, wherein the mineralizing solution comprises:
from about 1.5 mM to about 3.0 mM of phosphate ions; or
from about 1.8 mM to about 2.5 mM of phosphate ions.

28. The bone marrow model of claim 17, wherein the mineralizing solution comprises:
from about 3.0 mM to about 6.0 mM of calcium ions and from about 1.5 mM to about 3.0 mM of phosphate ions; or
from about 4.0 mM to about 5.0 mM of calcium ions and from about 1.8 mM to about 2.5 mM of phosphate ions.

29. The bone marrow model of claim 17, wherein the fluid medium comprises at least one agent selected from the group of antibiotic agents, antifungal agents, antiviral agents, buffers, anticoagulants, vitamins, salts, minerals, amino acids, nucleic acids, ribonucleic acids, fatty acids, lipids, O₂ gas, CO₂ gas, carbohydrates, serum proteins, cofactors, growth factors, cytokines, enzymes, hormones, signaling substances, and antibodies.

30. The bone marrow model of claim 17, wherein the fluid medium comprises from about 1× to about 20× phosphate buffered saline (PBS).

31. The bone marrow model claim 17, wherein the fluid medium comprises Dulbecco's Modified Eagle Medium (DMEM).

32. The bone marrow model of claim 17, wherein the fluid medium comprises from about 5× to about 20× phosphate buffered saline (PBS) and Dulbecco's Modified Eagle Medium (DMEM).

33. The bone marrow model of claim 17, wherein the at least one mineralizing solution comprises an inhibitor of hydroxyapatite formation, nucleation, or precipitation during the mineralizing process.

34. The bone marrow model of claim 33, wherein the inhibitor of hydroxyapatite formation comprises a protein capable of inhibiting hydroxyapatite nucleation and precipitation in solution.

35. The bone marrow model of claim 34, wherein the protein capable of inhibiting hydroxyapatite formation, nucleation, or precipitation is selected from the group consisting of Osteopontin (OPN), Osteocalcin (OC), Osteonectin (ON), bone sialoprotein (BSP), dentine phosphoryn (DPP), dentin matrix protein 1 (DMP1), dentin sialophosphoprotein (DSPP), matrix extracellular phosphoglycoprotein (MEPE), chondrocalcin (CC), Proline-rich protein 1 (PRP1), Proline-rich protein 2 (PRP2), Proline-rich protein 3 (PRP3), PRP1-T1, PRP3-T1, Histatin 5, MG1, MG2, Asialo_MG2, Amylase, statherin, cystatin S, cystatin SN, Cystatin S1, fetuin, and HSA.

36. The bone marrow model of claim 33, wherein the inhibitor of hydroxyapatite formation, nucleation, or precipitation comprises Osteopontin.

37. The bone marrow model of claim 36, wherein the osteopontin is present at a concentration of 50 μg/mL to 200 μg/mL.

38. The bone marrow model of claim 37, wherein the osteopontin is present at a concentration of 100 μg/mL.

39. The bone marrow model of claim 17, wherein the mineralizing solution comprises one or more sources of ionic minerals selected from the group of calcium phosphate, calcium carbonate, hydroxyapatite, strontium carbonate, barium carbonate, and calcium sulfate, strontium sulfate, calcium oxalate, magnesium-bearing calcium carbonate, and magnesium-bearing calcium phosphate.

40. The bone marrow model of claim 18, wherein the mineralizing solution is a calcifying solution.

41. The bone marrow model of claim 40, wherein the calcifying solution comprises calcium ions and phosphate ions.

42. A microfluidic bone marrow model comprising:
a central chamber open at two opposing ends and defined by continuous walls formed by an upper plate, a lower plate, and two side plates; wherein the upper plate and lower plate are substantially parallel to each other, are separated from one another by the height of the central chamber, and are substantially perpendicular to the two side plates; and wherein the two side plates are substantially parallel to each other, are separated from one another by the width of the central chamber, and are substantially perpendicular to the top plate and the bottom plate; and
a permeable barrier dividing the central chamber parallel to and maintained between the upper plate and lower plate and in contact with each of the two side plates, the permeable barrier dividing the central chamber into an upper chamber located between the permeable barrier and upper plate and a lower central chamber located between the permeable barrier and the lower plate;
wherein the lower central chamber is divided into a first microenvironment comprising an endosteal niche and a second microenvironment comprising a stem cell niche, the stem cell niche being in contact with the permeable barrier and the two side plates and the endosteal niche being in contact with the bottom plate and the two side plates;
and further wherein the stem cell niche and the endosteal niche are in contact and communication with each other.

43. The bone marrow model of any of claim 1, further comprising one or more cell selected from the group consisting of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages, and exosomes.

44. The bone marrow model of claim 1, wherein the second microenvironment comprises stem cells.

45. The bone marrow model of claim 1, wherein the second microenvironment comprises one or more of hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, endothelial cells (ECs), osteoblast precursor cells, osteolineage cells, pericytes, chondrocyte precursor cells, mesenchymal stem and progenitor cells or mesenchymal stromal progenitor cells, CXCL12-abundant reticular cells, and exosomes.

46. The bone marrow model of claim 1, wherein:
the first microenvironment comprises one or more types of diseased cells;
the second microenvironment comprises one or more types of diseased cells; or
both the first microenvironment and second microenvironment comprise one or more types of diseased cells.

47. The bone marrow model of claim 1, wherein the second microenvironment comprises cells found in hematopoietic niche experiencing or subject to a condition selected from the group consisting of hematologic malignancies, marrow failure syndromes, pre-malignant clonal hematopoiesis, and solid tumor metastases to bone marrow.

48. The bone marrow model of claim 44, wherein the one or more types of diseased cells are selected from acute myeloid leukemia, chronic myeloid leukemia, atypical chronic myeloid leukemia, chronic neutrophilic leukemia, acute lymphoblastic leukemia, multiple myeloma, Non-Hodgkin lymphoma, Chronic lymphocytic leukemia, Hodgkin lymphoma, T-cell lymphoma, bone marrow failure syndromes, myelodysplastic syndrome, clonal hematopoiesis of indeterminant potential, clonal cytopenias of undetermined significance, and aplastic anemia, and circulating tumor cells and cells of metastatic solid tumors that travel to the bone marrow

49. The bone marrow model of claim 48 wherein the cells of metastatic solid tumors that travel to the bone marrow are selected from the group of lung cancers, breast cancers, kidney cancers, prostate cancers, and thyroid cancers.

50. The bone marrow model of claim 1, wherein the first microenvironment further comprises cells selected from the group of osteoblasts, osteoprogenitors, and osteochondroprogenitors.

51. The bone marrow model of claim 1, wherein the first microenvironment further comprises osteoblasts.

52. The bone marrow model of claim 1, wherein the second microenvironment comprises cells selected from the group of mesenchymal stem cells and hematopoietic stem cells.

53. The bone marrow model of claim 1, wherein the first microenvironment comprises a mineralized collagen.

54. The bone marrow model of claim 1, wherein the first microenvironment and the second microenvironment are maintained in a position in which they can interact with a fluid medium.

55. The bone marrow model of claim 1, further comprising one or more fluid channels in communication with at least one of the first microenvironment and the second microenvironment.

56. The bone marrow model of claim 1, further comprising a fluid channel in communication with the first microenvironment.

57. The bone marrow model of claim 1, further comprising a fluid channel in communication with the second microenvironment.

58. The bone marrow model of claim 1, further comprising a fluid channel in communication with both the first microenvironment and the second microenvironment.

59. The bone marrow model of claim 1, further comprising a first fluid channel and a second fluid channel, wherein the first fluid channel is in communication with both the first microenvironment and the second microenvironment and the second fluid channel is in communication with both the first microenvironment and the second microenvironment.

60. The bone marrow model of claim 1, further comprising a first fluid channel and a second fluid channel, wherein the first fluid channel is in communication with both the first microenvironment and the second microenvironment and the second fluid channel is in communication with one of the first microenvironment and the second microenvironment.

61. The bone marrow model of claim 56, wherein epithelial cells line at least a portion of the first fluid channel, the second fluid channel, or both the first and the second fluid channels.

62. The bone marrow model of claim 56, wherein the vascular niche hydrogel contains endothelial cells.

63. A method of testing a drug or drug candidate, or a pharmaceutically acceptable salt thereof, using the bone marrow model of any claim 1, the method comprising:
ascertaining the elements of a device or design described in the bone marrow model having first microenvironment and a second microenvironment;
exposing at least one of the first microenvironment and the second microenvironment to the drug or drug candidate, or a pharmaceutically acceptable salt thereof; and
ascertaining any changes to the elements of the device or design following exposure to the drug or drug candidate, or a pharmaceutically acceptable salt thereof.

64. The method of claim 63, wherein the at least one of the first microenvironment and the second microenvironment is exposed to the drug or drug candidate, or a pharmaceutically acceptable salt thereof, by contact with at least one fluid media comprising the drug or drug candidate, or a pharmaceutically acceptable salt thereof.

65. The method of claim 63, wherein the method of drug testing is accomplished in vivo.

66. The method of claim 65 wherein the drug testing is accomplished in vivo using a ring, disk, or shard model.

67. The method of claim 63, wherein the drug testing is accomplished using one or more of: a high-throughput static assay; a high-throughput flow assay; or a high-throughput microfluidic device.

68. A system for use in a bone marrow model, comprising:
a well plate comprising:
an array of media wells configured to receive fluid media, a media well of the array of media wells including a bottom end that is covered with a permeable barrier;
an array of hydrogel chambers, a hydrogel chamber of the array of hydrogel chambers positioned underneath the permeable barrier; and
an array of loading ports, a loading port of the array of loading ports in fluid communication with the hydrogel chamber to direct a hydrogel containing cells into the hydrogel chamber.

69. The system of claim 68, wherein the loading port is positioned beside the media well, and wherein the hydrogel chamber is positioned underneath the loading port.

70. The system of claim 68, wherein a first number of the array of loading ports is greater than a second number of the array of media wells, wherein the loading port is a first loading port, and wherein a second loading port of the array of loading ports is in fluid communication with the hydrogel chamber.

71. The system of claim 68, wherein the loading port has a first end configured to receive a pipette tip and a second end at an opening to the hydrogel chamber, wherein the first end of the loading port has a first inner diameter, and wherein the second end of the loading port has a second inner diameter less than the first inner diameter.

72. The system of claim 68, wherein the well plate is a first well plate, wherein the media well further comprises a top end that opens into a recessed area at a top of the first well plate, wherein the first well plate further comprises a connector on an external side surface of the first well plate, and wherein the system further comprises a second well plate configured to detachably couple to the top of the first well plate, the second well plate comprising:
an array of tubes extending from a bottom surface of the second well plate, a tube of the array of tubes having an outer diameter less than an inner diameter of the media well to allow the tube to be inserted into the media well when the second well plate is coupled to the top of the first well plate; and
an array of collection wells in fluid communication with the array of tubes, a collection well of the array of collection wells positioned over the media well when the second well plate is coupled to the top of the first well plate,
wherein the recessed area at the top of the first well plate forms an air chamber when the second well plate is coupled to the top of the first well plate, and
wherein the air chamber is configured to be pressurized through operation of a pump that is connected to the connector.

73. The system of claim 72, wherein at least one of the first well plate or the second well plate includes an elastomer to create a hermetic seal when the second well plate is coupled to the top of the first well plate.

74. The system of claim 72, wherein the array of media wells are arranged in rows, wherein a row of the rows comprises multiple media wells, wherein each media well of the multiple media wells in the row is positioned adjacent to at least one loading port of the array of loading ports, wherein the row is separated from an adjacent row by a vertically-oriented wall to define the air chamber for the row of the multiple media wells, and wherein the connector allows for pressurizing the air chamber independently from other air chambers of other rows.

75. A system for use in a bone marrow model, comprising:
a well plate comprising:
a first media well and a second media well, the first media well and the second media well configured to receive fluid media, wherein a first bottom end of the first media well is covered with a permeable membrane, and wherein a second bottom end of the second media well is covered with the permeable membrane, or a different permeable membrane;
a first extracellular matrix (ECM) chamber and a second ECM chamber, wherein the first ECM chamber is positioned underneath the first media well with the permeable membrane interposed between the first ECM chamber and the first media well, and wherein the second ECM chamber is positioned underneath the second media well with the permeable membrane, or the different permeable membrane, interposed between the second ECM chamber and the second media well; and
a first loading port and a second loading port, wherein the first loading port is in fluid communication with the first ECM chamber, and wherein the second loading port is in fluid communication with the second ECM chamber.

76. The system of claim 75, wherein the first loading port is positioned beside the first media well, the first ECM chamber is positioned underneath the first loading port, the second loading port is beside the second media well, and the second ECM chamber is positioned underneath the second loading port.

77. The system of claim 75, wherein the well plate further includes a third loading port and a fourth loading port, wherein the third loading port is in fluid communication with the first ECM chamber, and wherein the fourth loading port is in fluid communication with the second ECM chamber.

78. The system of claim 75, wherein the first loading port has a first end configured to receive a pipette tip and a second end at an opening to the first ECM chamber, wherein the first end of the first loading port has a first inner diameter, and wherein the second end of the first loading port has a second inner diameter less than the first inner diameter.

79. The system of claim 75, wherein the well plate is a first well plate, wherein the first media well includes a first top end that opens into a first recessed area at a top of the first well plate, wherein the second media well includes a second top end that opens into a second recessed area at the top of the first well plate, wherein a vertically-oriented wall separates the first recessed area from the second recessed area, wherein the first well plate further comprises a connector and a second connector on an external side surface of the first well plate, and wherein the system further comprises a second well plate configured to detachably couple to the top of the first well plate, the second well plate comprising:
a first tube and a second tube extending from a bottom surface of the second well plate, wherein the first tube is configured to be inserted into the first media well and the second tube is configured to be inserted into the second media well when the second well plate is coupled to the top of the first well plate;
a first collection well in fluid communication with the first tube and positioned over the first media well when the second well plate is coupled to the top of the first well plate; and
a second collection well in fluid communication with the second tube and positioned over the second media well when the second well plate is coupled to the top of the first well plate,
wherein the first recessed area forms a first air chamber and the second recessed area forms a second air chamber when the second well plate is coupled to the top of the first well plate, and
wherein the first air chamber is configured to be pressurized through operation of a pump that is connected to the first connector and the second air chamber is configured to be pressurized through operation of the pump, or a different pump, that is connect to the second connector.

80. The system of claim 79, wherein at least one of the first well plate or the second well plate includes an elastomer to create a hermetic seal when the second well plate is coupled to the top of the first well plate.

81. A method comprising:
expressing, into an array of loading ports in a well plate, a hydrogel containing cells to at least partially fill an array of hydrogel chambers in the well plate with the hydrogel; and
filling, at least partially, an array of media wells in the well plate with fluid media,
wherein a media well of the array of media wells includes a bottom end that is covered with a permeable barrier,
wherein a hydrogel chamber of the array of hydrogel chambers is positioned underneath the permeable barrier, and
wherein the cells comprise one or more of osteoblasts, osteocytes, osteoclasts, mesenchymal stem cells, hematopoietic stem cells, stromal cells, endothelial cells, pericytes, neurons, HUVECs, myelopoietic cells, erythropoietic cells, megakaryocytes, plasma cells, reticular cells, lymphocytes, monocytes, adipocytes, fibroblasts, macrophages, hematopoietic stem cells (HSCs), long-term hematopoietic stem cells, short-term hematopoietic stem cells, multipotent progenitor cells, common myeloid progenitor cells, common lymphoid progenitor cells, megakaryocyte-erythroid progenitor cells, adipocytes, macrophages, granulocyte/macrophage progenitor cells, osteoblast precursor cells, osteolineage cells, chondrocyte precursor cells, mesenchymal stem and progenitor cells or mesenchymal stromal progenitor cells, CXCL12-abundant reticular cells, and exosomes.

82. The method of claim 81, further comprising:
mounting the well plate to an orbital shaker; and
operating the orbital shaker to agitate the fluid media and/or the hydrogel.

83. The method of claim 81, wherein the expressing the hydrogel to at least partially fill the hydrogel chamber comprises expressing the hydrogel from a pipette containing the hydrogel while a tip of the pipette is inserted into a loading port of the array of loading ports that is in fluid communication with the hydrogel chamber.

84. The method of claim 81, wherein the well plate is a first well plate, wherein a recessed area is defined in a top of the first well plate above one or more media wells of the array of media wells, and wherein the method further comprises
coupling a second well plate to the top of the first well plate to convert the recessed area into a hermetically-sealed air chamber;
coupling a pump to a connector on an external side surface of the first well plate; and
operating the pump to pressurize the hermetically-sealed air chamber as a pressurized air chamber,
wherein the second well plate comprises an array of tubes extending from a bottom surface of the second well plate;
wherein tubes in the array of tubes are in fluid communication with collection wells in an array of collection wells in the second well plate;
wherein one or more tubes of the array of tubes are inserted into the one or more media wells as a result of the coupling of the second well plate to the top of the first well plate; and
wherein the pressurized air chamber causes continuous perfusion of the fluid media through the one or more tubes.

85. The method of claim 84, wherein the pressurized air chamber is pressurized with a positive pressure or a negative pressure.

86. The method of claim 84, wherein the pressurized air chamber causes the fluid media to flow in an upward direction through the one or more tubes and into one or more collection wells of the array of collection wells.

87. The method of claim 81, wherein the well plate is a first well plate, wherein multiple recessed areas are defined in a top of the first well plate, the multiple recessed areas including a first recessed area above a first set of media wells of the array of media wells and a second recessed area above a second set of media wells of the array of media wells, and wherein the method further comprises
coupling a second well plate to the top of the first well plate to convert the first recessed area into a first hermetically-sealed air chamber and the second recessed area into a second hermetically-sealed air chamber;
coupling one or more pumps to a first connector and a second connector on an external side surface of the first well plate; and
operating the one or more pumps to pressurize the first hermetically-sealed air chamber as a first pressurized air chamber and the second hermetically-sealed as a second pressurized air chamber,
wherein the second well plate comprises an array of tubes extending from a bottom surface of the second well plate;
wherein tubes in the array of tubes are in fluid communication with collection wells in an array of collection wells in the second well plate;
wherein a first set of tubes of the array of tubes are inserted into the first set of media wells and a second set of tubes of the array of tubes are inserted into the second set of media wells as a result of the coupling of the second well plate to the top of the first well plate; and
wherein the first pressurized air chamber causes continuous perfusion of the fluid media through the first set of tubes and the second pressurized air chamber causes continuous perfusion of the fluid media through the second set of tubes.

88. A system for use in a bone marrow model, comprising:
a first reservoir comprising:
an array of microfluidic channels, a microfluidic channel of the array of microfluidic channels configured to receive fluid media at an inlet of the microfluidic channel and allow the fluid media to egress from an outlet of the microfluidic channel; and
an array of inserts, an insert of the array of inserts coupled to at least one of the inlet of the microfluidic channel or the outlet of the microfluidic channel and comprising:
a media channel to receive the fluid media at an inlet of the media channel and allow the fluid media to egress from an outlet of the media channel;
a hydrogel chamber positioned underneath the media channel, the hydrogel chamber configured to be filled, at least partially, with a hydrogel containing cells; and
a permeable barrier interposed between the hydrogel chamber and the media channel; and
a second reservoir configured to detachably couple to a bottom of the first reservoir, the second reservoir comprising:
an array of collection wells, a collection well of the array of collection wells positioned underneath the microfluidic channel when the second reservoir is coupled to the bottom of the first reservoir and configured to collect the fluid media that has passed through the microfluidic channel and through the insert.

89. The system of claim 88, wherein the first reservoir further comprises a recessed area defined in a top of the first reservoir to receive the fluid media, and wherein the inlet of the microfluidic channel is positioned at a bottom of the recessed area.

90. The system of claim 88, wherein the array of microfluidic channels comprises an array of spiral channels.

91. The system of claim 90, wherein an individual spiral channel of the array of spiral channels has:
a length that is no greater than about 11 millimeters; and
a diameter that is no greater than about 11 millimeters.

92. The system of claim 88, wherein the array of microfluidic channels comprises an array of serpentine channels.

93. The system of claim 92, wherein an individual serpentine channel of the array of serpentine channels has a width that is no greater than about 8 millimeters.

94. The system of claim 88, wherein:
the second reservoir further comprises a recessed section at a periphery of a top of the second reservoir;
the first reservoir further comprises a lip around a periphery of the bottom of the first reservoir; and
the second reservoir is configured to detachably couple to the bottom of the first reservoir by inserting the lip into the recessed section.

95. The system of claim 88, wherein the inlet of the media channel is positioned at a top of the insert at a first side of the insert, and wherein the outlet of the media channel is positioned at a bottom of the insert at a second side of the insert opposite the first side.

96. A system for use in a bone marrow model, comprising:
a first reservoir comprising:
a first microfluidic channel and a second microfluidic channel, the first microfluidic channel and the second microfluidic channel each configured to allow fluid media to pass therethrough; and
a first insert and a second insert, the first insert coupled to at least one of an inlet of the first microfluidic channel or an outlet of the first microfluidic channel, and the second insert coupled to at least one of an inlet of the second microfluidic channel or an outlet of the second microfluidic channel, the first insert and the second insert each comprising:
a media channel to receive the fluid media at an inlet of the media channel and allow the fluid media to egress from an outlet of the media channel;
an extracellular matrix (ECM) chamber positioned underneath the media channel, the ECM chamber configured to be filled, at least partially, with an ECM; and
a permeable barrier interposed between the ECM chamber and the media channel; and
a second reservoir comprising:
a first collection well and a second collection well, wherein the first collection well is vertically-aligned with the first microfluidic channel and the second collection well is vertically-aligned with the second microfluidic channel when the second reservoir is positioned underneath the first reservoir.

97. The system of claim 96, wherein the first reservoir further comprises a recessed area defined in a top of the first reservoir to receive the fluid media, and wherein the inlet of the first microfluidic channel and the inlet of the second microfluidic channel are positioned at a bottom of the recessed area.

98. The system of claim 96, wherein at least one of the first microfluidic channel or the second microfluidic channel comprises a spiral channel.

99. The system of claim 98, wherein the spiral channel has:
a length that is no greater than about 11 millimeters; and
a diameter that is no greater than about 11 millimeters.

100. The system of claim 96, wherein at least one of the first microfluidic channel or the second microfluidic channel comprises a serpentine channel.

101. The system of claim 100, wherein the serpentine channel has a width that is no greater than about 8 millimeters.

102. The system of claim 96, wherein:
the second reservoir further comprises a recessed section at a periphery of a top of the second reservoir; and
the first reservoir further comprises a lip around a periphery of the bottom of the first reservoir, the lip configured to be inserted into the recessed section when the first reservoir is set on top of the second reservoir.

103. The system of claim 96, wherein the inlet of the media channel is positioned at a top of the first insert and on a first side of the first insert, and wherein the outlet of the media channel is positioned at a bottom of the first insert and on a second side of the first insert, the second side opposite the first side.

104. A vacuum insert for use in a bone marrow model, comprising:
an annular base comprising:
a plurality of inlets defined in a top of the annular base;
a media channel defined in an interior of the annular base and in fluid communication with the plurality of inlets; and
a permeable barrier positioned underneath the media channel; and
a central tube coupled to the annular base at a center of the annular base and extending orthogonally from the top of the annular base,
wherein the vacuum insert is configured to couple to a vacuum source at a top end of the central tube, and wherein the media channel of the annular base is configured to allow fluid media drawn into the vacuum insert via the plurality of inlets to pass through the annular base and into the central tube.

105. The vacuum insert of claim 104, wherein the vacuum insert is configured to be placed inside a media well of a well plate with the annular base at a bottom of the media well.

106. The vacuum insert of claim 104, wherein the permeable barrier spans an area of the annular base.

107. The vacuum insert of claim 104, wherein the annular base comprises a recessed area defined in a bottom of the annular base to accommodate a hydrogel containing cells underneath the permeable barrier.

108. The vacuum insert of claim 104, wherein the annular base further comprises a hydrogel chamber defined in the annular base underneath the permeable barrier, the hydrogel chamber configured to be loaded with hydrogel containing cells.

109. The vacuum insert of claim 104, wherein the plurality of inlets are spaced equidistantly around the periphery of the annular base.

110. A vacuum insert for use in a bone marrow model, comprising:
an annular base comprising:
a one or more inlets defined in a top of the annular base;
a media channel defined in an interior of the annular base and in fluid communication with the one or more inlets; and
a permeable barrier positioned underneath the media channel; and
a central tube at a center of the annular base and extending orthogonally from the top of the annular base,
wherein the vacuum insert is configured to couple to a vacuum source at a top end of the central tube, and
wherein the central tube is in fluid communication with the media channel.

111. The vacuum insert of claim 110, wherein the vacuum insert is configured to be placed inside a media well of a well plate.

112. The vacuum insert of claim 111, wherein the vacuum insert is configured to be aspirated while other vacuum inserts within other media wells of the well plate are being aspirated via the vacuum source.

113. The vacuum insert of claim 110, wherein the permeable barrier spans an area of the annular base.

114. The vacuum insert of claim 110, wherein the annular base further comprises a recessed area defined in a bottom of the annular base to accommodate a hydrogel containing cells underneath the permeable barrier.

115. The vacuum insert of claim 110, wherein the annular base further comprises a hydrogel chamber defined in the annular base underneath the permeable barrier, the hydrogel chamber configured to be loaded with hydrogel containing cells.

116. The vacuum insert of claim 110, wherein the one or more inlets comprise a plurality of inlets spaced equidistantly around the periphery of the annular base.

117. A microfluidic chip for use in a bone marrow model, comprising:
a first substrate comprising:
an inlet defined in a top of the first substrate, the inlet configured to allow fluid media to ingress into the microfluidic chip;
an outlet defined in the top of the first substrate, the outlet configured to allow the fluid media to egress from the microfluidic chip; and
a loading port defined in the top of the first substrate, the loading port configured to receive a hydrogel containing cells;
a second substrate disposed underneath the first substrate, the second substrate comprising:
a hydrogel channel configured to be filled, at least partially, with the hydrogel containing cells, wherein an end of the hydrogel channel is vertically-aligned with the loading port;
a first through-hole that is vertically-aligned with the inlet; and
a second through-hole that is vertically-aligned with the outlet;
a permeable barrier disposed underneath the second substrate; and
a third substrate disposed underneath the permeable barrier, the third substrate comprising:
a media channel configured to receive the fluid media, wherein a first end of the media channel is vertically-aligned with the first through-hole and a second end of the media channel is vertically-aligned with the second through-hole.

118. The microfluidic chip of claim 117, wherein the media channel spans a center of the third substrate and is oriented horizontally on the third substrate.

119. The microfluidic chip of claim 117, wherein the hydrogel channel spans a center of the second substrate and is oriented horizontally on the second substrate.

120. The microfluidic chip of claim 117, wherein:
a center portion of the hydrogel channel is straight; and
a peripheral portion of the hydrogel channel is curved.

121. The microfluidic chip of claim 117, wherein:
the first substrate further comprises a second loading port defined in the top of the first substrate; and
the hydrogel channel comprises a second end that is vertically-aligned with the second loading port.

122. The microfluidic chip of claim 117, wherein:
the first substrate further comprises:
a second inlet defined in the top of the first substrate, the second inlet configured to allow the fluid media to ingress into the microfluidic chip;
a second outlet defined in the top of the first substrate, the second outlet configured to allow the fluid media to egress from the microfluidic chip; and
a second loading port defined in the top of the first substrate, the second loading port configured to receive the hydrogel containing cells;
the second substrate further comprises:
a second hydrogel channel configured to be filled, at least partially, with the hydrogel containing cells, wherein an end of the second hydrogel channel is vertically-aligned with the second loading port;
a third through-hole that is vertically-aligned with the second inlet; and
a fourth through-hole that is vertically-aligned with the second outlet; and
the third substrate further comprises:
a second media channel configured to receive the fluid media, wherein a first end of the second media channel is vertically-aligned with the third through-hole and a second end of the second media channel is vertically-aligned with the fourth through-hole.

123. The microfluidic chip of claim 122, wherein:
the inlet and the second inlet are spaced along a first side edge of the first substrate; and
the outlet and the second outlet are positioned at a second side edge of the first substrate, the second side edge opposite the first side edge.

124. A device for use in a bone marrow model, comprising:
a top substrate comprising:
an inlet defined in a top of the top substrate, the inlet configured to allow fluid media to ingress into the device;
an outlet defined in the top of the top substrate, the outlet configured to allow the fluid media to egress from the device; and
a loading port defined in the top of the top substrate, the loading port configured to receive a hydrogel containing cells;
a bottom substrate comprising a media channel configured to receive the fluid media;
an intermediate substrate interposed between the top substrate and the bottom substrate, the intermediate substrate comprising:
a hydrogel channel configured to be filled, at least partially, with the hydrogel containing cells, wherein an end of the hydrogel channel is vertically-aligned with the loading port;
a first through-hole that is vertically-aligned with the inlet and with a first end of the media channel; and
a second through-hole that is vertically-aligned with the outlet and with a second end of the media channel; and
a permeable barrier interposed between the intermediate substrate and the bottom substrate.

125. The device of claim 124, wherein the media channel spans a center of the bottom substrate and is oriented horizontally on the bottom substrate.

126. The device of claim 124, wherein the hydrogel channel spans a center of the intermediate substrate and is oriented horizontally on the intermediate substrate.

127. The device of claim 124, wherein:
a center portion of the hydrogel channel is straight; and
a peripheral portion of the hydrogel channel is curved.

128. The device of claim 124, wherein:
the top substrate further comprises a second loading port defined in the top of the top substrate; and
the hydrogel channel comprises a second end that is vertically-aligned with the second loading port.

129. The device of claim 124, wherein:
the top substrate further comprises:
a second inlet defined in the top of the top substrate, the second inlet configured to allow the fluid media to ingress into the device;
a second outlet defined in the top of the top substrate, the second outlet configured to allow the fluid media to egress from the device; and
a second loading port defined in the top of the top substrate, the second loading port configured to receive the hydrogel containing cells;
the bottom substrate further comprises a second media channel configured to receive the fluid media; and
the intermediate substrate further comprises:
a second hydrogel channel configured to be filled, at least partially, with the hydrogel containing cells, wherein an end of the second hydrogel channel is vertically-aligned with the second loading port;
a third through-hole that is vertically-aligned with the second inlet and with a first end of the second media channel; and
a fourth through-hole that is vertically-aligned with the second outlet and with a second end of the second media channel.

130. The device of claim 129, wherein:
the inlet and the second inlet are spaced along a first side edge of the top substrate; and
the outlet and the second outlet are positioned at a second side edge of the top substrate, the second side edge opposite the first side edge.
